SEQUENCE LISTING (165 sequences)

SEQ ID NO: 1 - DNA, 21 bp, synthetic construct
  source 1..21: mol_type = other DNA
gatccaccggtcgccaccatg

SEQ ID NO: 2 - DNA, 20 bp, synthetic construct
  source 1..20: mol_type = other DNA
gtgttctgctggtagtggtc

SEQ ID NO: 3 - DNA, 19 bp, synthetic construct
  source 1..19: mol_type = other DNA
gggtgtcggggctggctta

SEQ ID NO: 4 - DNA, 21 bp, synthetic construct
  source 1..21: mol_type = other DNA
caatttcacacaggaaacagc

SEQ ID NO: 5 - DNA, 72 bp, synthetic construct
  source 1..72: mol_type = other DNA
gcgaattaatacgactcactatagggcttaagtataaggaggaaaaaatatggtgagcaagggcgaggagct

SEQ ID NO: 6 - DNA, 56 bp, synthetic construct
  source 1..56: mol_type = other DNA
aaacccctccgtttagagaggggttatgctagttattacttgtacagctcgtccat

SEQ ID NO: 7 - DNA, 69 bp, synthetic construct
  source 1..69: mol_type = other DNA
gcgaattaatacgactcactatagggcttaagtataaggaggaaaaaatatgatcagtctgattgcggc

SEQ ID NO: 8 - DNA, 54 bp, synthetic construct
  source 1..54: mol_type = other DNA
aaacccctccgtttagagaggggttatgctagttattaccgccgctccagaatc

SEQ ID NO: 9 - DNA, 90 bp, synthetic construct
  source 1..90: mol_type = other DNA
gcgaattaatacgactcactatagggcttaagtataaggaggaaaaaatatgcaccaccaccaccaccacgtgtctaagggagaagaact

SEQ ID NO: 10 - DNA, 60 bp, synthetic construct
  source 1..60: mol_type = other DNA
aaacccctccgtttagagaggggttatgctagttatcacttatataattcgtccatacca

SEQ ID NO: 11 - DNA, 18 bp, synthetic construct
  source 1..18: mol_type = other DNA
tgtaaaacgacggccagt

SEQ ID NO: 12 - DNA, 18 bp, synthetic construct
  source 1..18: mol_type = other DNA
caggaaacagctatgacc

SEQ ID NO: 13 - DNA, 54 bp, synthetic construct
  source 1..54: mol_type = other DNA
acactctttccctacacgacgctcttccgatctggaggacaaagtacaaacggc

SEQ ID NO: 14 - DNA, 52 bp, synthetic construct
  source 1..52: mol_type = other DNA
gactggagttcagacgtgtgctcttccgatctatcgatgtcctccccattgg

SEQ ID NO: 15 - DNA, 22 bp, synthetic construct
  source 1..22: mol_type = other DNA
ttggaccctcgtacagaagcta

SEQ ID NO: 16 - DNA, 25 bp, synthetic construct
  source 1..25: mol_type = other DNA
ttcttcctactcaggctttattcaa

SEQ ID NO: 17 - DNA, 20 bp, synthetic construct
  source 1..20: mol_type = other DNA
ccacctgggtcgacattgat

SEQ ID NO: 18 - DNA, 20 bp, synthetic construct
  source 1..20: mol_type = other DNA
tgtccttccgagtgagagac

SEQ ID NO: 19 - DNA, 21 bp, synthetic construct
  source 1..21: mol_type = other DNA
atggtgagcaagggcgaggag

SEQ ID NO: 20 - DNA, 24 bp, synthetic construct
  source 1..24: mol_type = other DNA
ttacttgtacagctcgtccatgcc

SEQ ID NO: 21 - DNA, 20 bp, synthetic construct
  source 1..20: mol_type = other DNA
ccatccccttctgtgaatgt

SEQ ID NO: 22 - DNA, 20 bp, synthetic construct
  source 1..20: mol_type = other DNA
ggagattggagacacggaga

SEQ ID NO: 23 - DNA, 16 bp, synthetic construct
  source 1..16: mol_type = other DNA
gctgaccctgaagttc

SEQ ID NO: 24 - DNA, 16 bp, synthetic construct
  source 1..16: mol_type = other DNA
gaacttcagggtcagc

SEQ ID NO: 25 - DNA, 16 bp, synthetic construct
  source 1..16: mol_type = other DNA
  modified_base 2: mod_base = OTHER; note = 2-aminoadenine
  modified_base 3: mod_base = OTHER; note = 2-aminoadenine
  modified_base 8: mod_base = OTHER; note = 2-aminoadenine
  modified_base 14: mod_base = OTHER; note = 2-aminoadenine
gnncttcngggtcngc

SEQ ID NO: 26 - DNA, 18 bp, synthetic construct
  source 1..18: mol_type = other DNA
gacggcagcgtgcagctc

SEQ ID NO: 27 - DNA, 18 bp, synthetic construct
  source 1..18: mol_type = other DNA
gagctgcacgctgccgtc

SEQ ID NO: 28 - DNA, 18 bp, synthetic construct
  source 1..18: mol_type = other DNA
  modified_base 2: mod_base = OTHER; note = 2-aminoadenine
  modified_base 8: mod_base = OTHER; note = 2-aminoadenine
gngctgcncgctgccgtc

SEQ ID NO: 29 - DNA, 45 bp, synthetic construct
  source 1..45: mol_type = other DNA
gcgggcccgggatccaccggtatggaagacgccaaaaacataaag

SEQ ID NO: 30 - DNA, 47 bp, synthetic construct
  source 1..47: mol_type = other DNA
cagcctgcacctgaggagtgcggccgcttacacggcgatctttccgc

SEQ ID NO: 31 - DNA, 20 bp, synthetic construct
  source 1..20: mol_type = other DNA
gtcttgtagttgccgtcgtc

SEQ ID NO: 32 - DNA, 114 bp, synthetic construct
  source 1..114: mol_type = other DNA
taatacgactcactataggctgaagggcatcgacttcagttttagagctagaaatagcaagttaaaataaggctagtccgttatcaacttgaaaaagtggcaccgagtcggtgc

SEQ ID NO: 33 - DNA, 58 bp, synthetic construct
  source 1..58: mol_type = other DNA
taatacgactcactataggaatttctactgttgtagatcgtcgccgtccagctcgacc

SEQ ID NO: 34 - DNA, 58 bp, synthetic construct
  source 1..58: mol_type = other DNA
taatacgactcactataggaatttctactgttgtagattttaccttcttcttgatatg

SEQ ID NO: 35 - DNA, 58 bp, synthetic construct
  source 1..58: mol_type = other DNA
taatacgactcactataggaatttctactgttgtagataactatattttttcacatag

SEQ ID NO: 36 - DNA, 58 bp, synthetic construct
  source 1..58: mol_type = other DNA
taatacgactcactataggaatttctactgttgtagataaggccaaacctcggcttac

SEQ ID NO: 37 - DNA, 58 bp, synthetic construct
  source 1..58: mol_type = other DNA
taatacgactcactataggaatttctactgttgtagatcactaacacgcatatttgat

SEQ ID NO: 38 - DNA, 58 bp, synthetic construct
  source 1..58: mol_type = other DNA
taatacgactcactataggaatttctactgttgtagattcagatattctactttgttt

SEQ ID NO: 39 - DNA, 58 bp, synthetic construct
  source 1..58: mol_type = other DNA
taatacgactcactataggaatttctactgttgtagatcccagagtccttcaaagata

SEQ ID NO: 40 - DNA, 58 bp, synthetic construct
  source 1..58: mol_type = other DNA
taatacgactcactataggaatttctactgttgtagatagttgtagatatgatttagc

SEQ ID NO: 41 - DNA, 58 bp, synthetic construct
  source 1..58: mol_type = other DNA
taatacgactcactataggaatttctactgttgtagatttcatatgttcttctagctt

SEQ ID NO: 42 - DNA, 58 bp, synthetic construct
  source 1..58: mol_type = other DNA
taatacgactcactataggaatttctactgttgtagatctgaatatctatgcattaat

SEQ ID NO: 43 - DNA, 58 bp, synthetic construct
  source 1..58: mol_type = other DNA
taatacgactcactataggaatttctactgttgtagatttatatatgtgatacatttt

SEQ ID NO: 44 - DNA, 58 bp, synthetic construct
  source 1..58: mol_type = other DNA
taatacgactcactataggaatttctactgttgtagatgaatcaacaaaaaaattagt

SEQ ID NO: 45 - DNA, 58 bp, synthetic construct
  source 1..58: mol_type = other DNA
taatacgactcactataggaatttctactgttgtagatagatttaaaaagaataagta

SEQ ID NO: 46 - DNA, 58 bp, synthetic construct
  source 1..58: mol_type = other DNA
taatacgactcactataggaatttctactgttgtagataaagagcaacaaaatggctt

SEQ ID NO: 47 - DNA, 61 bp, synthetic construct
  source 1..61: mol_type = other DNA
taatacgactcactataggaatttctactgttgtagattggttgcccaccctagtcattgg

SEQ ID NO: 48 - DNA, 61 bp, synthetic construct
  source 1..61: mol_type = other DNA
taatacgactcactatagggagtccgagcagaagaagaagttttagagctatgctgttttg

SEQ ID NO: 49 - DNA, 105 bp, synthetic construct
  source 1..105: mol_type = other DNA
taatacgactcactataggggaaccattcaaaacagcatagcaagttaaaataaggctagtccgttatcaacttgaaaaagtggcaccgagtcggtgcttttttt

SEQ ID NO: 50 - DNA, 115 bp, synthetic construct
  source 1..115: mol_type = other DNA
taatacgactcactatagggagtccgagcagaagaagaagttttagagctagaaatagcaagttaaaataaggctagtccgttatcaacttgaaaaagtggcaccgagtcggtgc

SEQ ID NO: 51 - DNA, 61 bp, synthetic construct
  source 1..61: mol_type = other DNA
taatacgactcactataggaataatttctattatattacgttttagagctatgctgttttg

SEQ ID NO: 52 - DNA, 61 bp, synthetic construct
  source 1..61: mol_type = other DNA
taatacgactcactatagggagtgacactgtgaaggagagttttagagctatgctgttttg

SEQ ID NO: 53 - DNA, 61 bp, synthetic construct
  source 1..61: mol_type = other DNA
taatacgactcactatagggtcaagaaagcagagactgcgttttagagctatgctgttttg

SEQ ID NO: 54 - DNA, 61 bp, synthetic construct
  source 1..61: mol_type = other DNA
taatacgactcactatagggtaaacaaagcatagactgagttttagagctatgctgttttg

SEQ ID NO: 55 - DNA, 61 bp, synthetic construct
  source 1..61: mol_type = other DNA
taatacgactcactatagggaagaccaaggatagactgcgttttagagctatgctgttttg

SEQ ID NO: 56 - DNA, 61 bp, synthetic construct
  source 1..61: mol_type = other DNA
taatacgactcactatagggaacataaagaatagaatgagttttagagctatgctgttttg

SEQ ID NO: 57 - DNA, 61 bp, synthetic construct
  source 1..61: mol_type = other DNA
taatacgactcactatagggaacacaaagcatagactgcgttttagagctatgctgttttg

SEQ ID NO: 58 - DNA, 55 bp, synthetic construct
  source 1..55: mol_type = other DNA
acactctttccctacacgacgctcttccgatctgcctcctgagtttctcatctgt

SEQ ID NO: 59 - DNA, 54 bp, synthetic construct
  source 1..54: mol_type = other DNA
gactggagttcagacgtgtgctcttccgatctccctagtcattggaggtgacat

SEQ ID NO: 60 - DNA, 22 bp, synthetic construct
  source 1..22: mol_type = other DNA
tcctcaccttcagcagctcctc

SEQ ID NO: 61 - DNA, 26 bp, synthetic construct
  source 1..26: mol_type = other DNA
gtggcaggcagattatcattcccatc

SEQ ID NO: 62 - DNA, 34 bp, synthetic construct
  source 1..34: mol_type = other DNA
catacttaaagtaaagcagaaggaataacagtgc

SEQ ID NO: 63 - DNA, 31 bp, synthetic construct
  source 1..31: mol_type = other DNA
gaagtttcattccactatagaactggttctg

SEQ ID NO: 64 - DNA, 26 bp, synthetic construct
  source 1..26: mol_type = other DNA
gagtactctccaatgacacctttggc

SEQ ID NO: 65 - DNA, 24 bp, synthetic construct
  source 1..24: mol_type = other DNA
atcatagcgagaccctgtctcagc

SEQ ID NO: 66 - DNA, 33 bp, synthetic construct
  source 1..33: mol_type = other DNA
caactaaagtcactctgctgtgctattagatac

SEQ ID NO: 67 - DNA, 30 bp, synthetic construct
  source 1..30: mol_type = other DNA
cagaaagacaagtttcacatgttctcactc

SEQ ID NO: 68 - DNA, 25 bp, synthetic construct
  source 1..25: mol_type = other DNA
acaagacctggctgagctaactgtg

SEQ ID NO: 69 - DNA, 23 bp, synthetic construct
  source 1..23: mol_type = other DNA
ccagccccatctgtcaaactgtg

SEQ ID NO: 70 - DNA, 26 bp, synthetic construct
  source 1..26: mol_type = other DNA
cacctgggcgagaaaggtaacttatg

SEQ ID NO: 71 - DNA, 24 bp, synthetic construct
  source 1..24: mol_type = other DNA
cagtgttggctttcacaaggatgc

SEQ ID NO: 72 - DNA, 23 bp, synthetic construct
  source 1..23: mol_type = other DNA
agaaggaggtgcaggagctagac

SEQ ID NO: 73 - DNA, 25 bp, synthetic construct
  source 1..25: mol_type = other DNA
ccctcagccactttatttcatcccc

SEQ ID NO: 74 - DNA, 21 bp, synthetic construct
  source 1..21: mol_type = other DNA
gtgtggagagtgagtaagcca

SEQ ID NO: 75 - DNA, 21 bp, synthetic construct
  source 1..21: mol_type = other DNA
acggtaggatgatttcaggca

SEQ ID NO: 76 - DNA, 21 bp, synthetic construct
  source 1..21: mol_type = other DNA
cacaaagcagtgtagctcagg

SEQ ID NO: 77 - DNA, 27 bp, synthetic construct
  source 1..27: mol_type = other DNA
cttttttggtactcgagtgttattcag

SEQ ID NO: 78 - DNA, 480 bp, synthetic construct
  source 1..480: mol_type = other DNA
atgatcagtctgattgcggcgttagcggtagatcgcgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcgtgtggtgacgtaccagaaatcatggtgattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcggtaa

SEQ ID NO: 79 - DNA, 20 bp, synthetic construct
  source 1..20: mol_type = other DNA
tcagatattctactttgttt

SEQ ID NO: 80 - DNA, 20 bp, synthetic construct
  source 1..20: mol_type = other DNA
aactatattttttcacatag

SEQ ID NO: 81 - DNA, 20 bp, synthetic construct
  source 1..20: mol_type = other DNA
cccagagtccttcaaagata

SEQ ID NO: 82 - DNA, 20 bp, synthetic construct
  source 1..20: mol_type = other DNA
agttgtagatatgatttagc

SEQ ID NO: 83 - DNA, 20 bp, synthetic construct
  source 1..20: mol_type = other DNA
aaggccaaacctcggcttac

SEQ ID NO: 84 - DNA, 20 bp, synthetic construct
  source 1..20: mol_type = other DNA
ttatatatgtgatacatttt

SEQ ID NO: 85 - DNA, 3839 bp, synthetic construct
  source 1..3839: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagataattaatagaagtcaatgtagggaaggaaatatggcagaaattaaacatatattcataattcaaaagtgaaatatggtagtgaaaacatatgtctgccatatatataatatatgcatatgtaatacatgtgtatatatgtatgctgtgagctaaatcatatctacaacttaaaaaatgaacataatggaataaggatattttaaattattatgttaaaatgttaagtatacttggagttggtgaaaaaaaatcaattcatttatacagacaatccaagaaggtatgacacattactgttttcataggaaaaataggcaagttgcaatcctttgaaatagatttggcttttgatatcatcaatatctttgaaggactctgggtaaaatatctgtttcccatcacattttccaattaattgctatgtgaaaaaatatagtttaaaggccaaacctcggcttacctgaaattttcgaagtttattcatatgttcttctagcttttggcagctttccaccaactgggaggaaagtttcttccagtgcccctcaatctcttcaaattctgacagatatttctggcatatttctgaaggtgctttcttggccatctccttcacagtgtcactcagatagttgaagccattttgttgctctttcaaagaactttgcagagcctcaaaattaaatagaagttcatttacactaacacgcatatttgatgagtttcattcatatcaagaagaaggtaaataaaagtttatgtaatttagagatctaaacaaagtagaatatctgataaagcaatatatatgtattcttatgaataatttctattatattacagggcatattatatttagacagaaatttagaagtcataattttactgaatatctatgcattaataactatgtttattatatatgtgatacattttaacttacctacagtgaaacattattaagacatttaggcaatatatgattaatatattttcagatttaaaaagaataagtatacaacatgggatttttagaatcaacaaaaaaattagtctttatatgtcctcacatcacagaagtttctcatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 86 - DNA, 720 bp, synthetic construct
  source 1..720: mol_type = other DNA
atggtgtctaagggagaagaactgtttaccggggtagtgcccattcttgtcgagcttgacggtgacgtaaatggccataagttctcagtttctggagagggcgagggagacgctacgtacgggaagttgacactgaaattcatttgcacgacgggtaaattgcctgtgccttggcccaccttagtcacaacattaacgtacggggtgcagtgcttcagtcgctacccagatcacatgaagcaacacgacttcttcaaatcagccatgcctgaaggatacgtgcaggaacgtacaatcttcttcaaggatgacggcaattacaagacccgtgcggaagttaaatttgagggcgacacacttgtgaatcgcatcgaattgaaaggtattgatttcaaagaagatgggaatatccttgggcataagttggaatataactacaacagtcacaatgtttacatcatggcagataagcagaaaaacggtattaaggtaaatttcaaaattcgccataacattgaggatggttcggttcaacttgccgatcactaccagcaaaacacgcctattggggatggaccagttcttctgcccgataatcattacctgagcacgcaatcggctttgtcaaaagacccaaacgaaaaacgtgaccatatggtcttgcttgagttcgtaacggccgccggtattacgcttggtatggacgaattatataagtga

SEQ ID NO: 87 - DNA, 503 bp, synthetic construct
  source 1..503: mol_type = other DNA
gtaaaacgacggccagtgcaacgcgatgacgatggatagcgattcatcgatgagctgacccgatcgccgccgccggagggttgcgtttgagacgggcgacagatagcggccgcgaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgtctctcactcggaaggacatatgggagggcaaatcatttaaaacatcagaatgagtatttggtttagagtttggcaacatatgccatcagttctggaccagcgagctgtgctgcgactcgtggcgtaatcatggtcatagctgtttcctg

SEQ ID NO: 88 - DNA, 702 bp, synthetic construct
  source 1..702: mol_type = other DNA
gtaaaacgacggccagtgcaacgcgatgacgatggatagcgattcatcgatgagctgacccgatcgccgccgccggagggttgcgtttgagacgggcgacagatctacgtattagtcatcgctattaccatggtggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggggtttagtgaaccgtcagatccgctagcgctaccggactcagatctcgagctcaagcttcgaattctgcagtcgacggtaccgcgggcccgggatccaccggtcatcagttctggaccagcgagctgtgctgcgactcgtggcgtaatcatggtcatagctgtttcctg

SEQ ID NO: 89 - DNA, 513 bp, synthetic construct
  source 1..513: mol_type = other DNA
gtaaaacgacggccagtgcaacgcgatgacgatggatagcgattcatcgatgagctgacccgatcgccgccgccggagggttgcgtttgagacgggcgacagatctacgtattagtcatcgctattaccatggggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggtttagtgaaccgtcagatccgctagcgctaccggactcagatctcgagctcaagcttcgaattctgcagtcgacggtaccgcgggcccgggatccaccggtcatcagttctggaccagcgagctgtgctgcgactcgtggcgtaatcatggtcatagctgtttcctg

SEQ ID NO: 90 - DNA, 808 bp, synthetic construct
  source 1..808: mol_type = other DNA
gtaaaacgacggccagtgcaacgcgatgacgatggatagcgattcatcgatgagctgacccgatcgccgccgccggagggttgcgtttgagacgggcgacagatccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctccatcagttctggaccagcgagctgtgctgcgactcgtggcgtaatcatggtcatagctgtttcctg

SEQ ID NO: 91 - DNA, 213 bp, synthetic construct
  source 1..213: mol_type = other DNA
gcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactaggg

SEQ ID NO: 92 - DNA, 128 bp, synthetic construct
  source 1..128: mol_type = other DNA
ggaggacaaagtacaaacggcagaagctggaggaggaagggcctgagtccgagcagaagaagaagggctcccatcacatcaaccggtggcgcattgccacgaagcaggccaatggggaggacatcgat

SEQ ID NO: 93 - DNA, 1640 bp, synthetic construct
  source 1..1640: mol_type = other DNA
ccacctgggtcgacattgattattgactagtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggaca

SEQ ID NO: 94 - DNA, 1670 bp, synthetic construct
  source 1..1670: mol_type = other DNA
ccacctgggtcgacattgattattgactagttgttacataacttatggtaaatggcctgcctggctgactgcccaatgacccctgcccattgatgtcaataatgatgtatgttcccatagtaatgccaatagggactttccattgatgtcaatgggtggagtatttatggtaaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacagaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggaca

SEQ ID NO: 95 - DNA, 1670 bp, synthetic construct
  source 1..1670: mol_type = other DNA
ccacctgggtcgacattgattattgactagttgttacataacttatggtaaatggcctgcctggctgactgcccaatgacccctgcccattgatgtcaataatgatgtatgttcccatagtaatgccaatagggactttccattgatgtcaatgggtggagtatttatggtaaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacagaccggtcgccaccatggtttctaagggagaagaactctttactggtgttgtcccaattctggttgagctggatggtgatgtgaatggccacaaattctctgtgtctggtgaaggtgaaggagatgcaacttatggaaagctgactctgaagttcatttgtacaacaggaaagctgccagtgccttggccaactctggtgaccaccctgacttatggtgttcaatgtttcagcaggtaccctgaccacatgaagcagcatgacttctttaaatctgcaatgccagaaggttatgttcaggagaggacaatcttctttaaggatgatggaaattataagacaagggcagaagtgaagtttgaaggtgatacactggttaacagaattgagctgaaaggcattgattttaaggaagatggaaacattctgggtcacaagctggagtacaactataattctcacaatgtttacattatggcagataagcagaagaatggaattaaggttaatttcaagattagacacaacattgaggatggatctgtccaactggcagaccattaccagcagaacacccctattggtgatggcccagttctcctcccagataatcactatctcagcactcaatctgctctgtccaaagaccctaatgagaaaagagaccacatggtcctcctggagtttgtgacagcagcaggaattactctgggaatggatgagctgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggaca

SEQ ID NO: 96 - DNA, 4479 bp, synthetic construct
  source 1..4479: mol_type = other DNA
ctagtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattga

SEQ ID NO: 97 - DNA, 3472 bp, synthetic construct
  source 1..3472: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatatggtgtctaagggagaagaactgtttaccggggtagtgcccattcttgtcgagcttgacggtgacgtaaatggccataagttctcagtttctggagagggcgagggagacgctacgtacgggaagttgacactgaaattcatttgcacgacgggtaaattgcctgtgccttggcccaccttagtcacaacattaacgtacggggtgcagtgcttcagtcgctacccagatcacatgaagcaacacgacttcttcaaatcagccatgcctgaaggatacgtgcaggaacgtacaatcttcttcaaggatgacggcaattacaagacccgtgcggaagttaaatttgagggcgacacacttgtgaatcgcatcgaattgaaaggtattgatttcaaagaagatgggaatatccttgggcataagttggaatataactacaacagtcacaatgtttacatcatggcagataagcagaaaaacggtattaaggtaaatttcaaaattcgccataacattgaggatggttcggttcaacttgccgatcactaccagcaaaacacgcctattggggatggaccagttcttctgcccgataatcattacctgagcacgcaatcggctttgtcaaaagacccaaacgaaaaacgtgaccatatggtcttgcttgagttcgtaacggccgccggtattacgcttggtatggacgaattatataagtgaatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 98 - DNA, 804 bp, synthetic construct
  source 1..804: mol_type = other DNA
gcgaattaatacgactcactatagggcttaagtataaggaggaaaaaatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataactagcataacccctctctaaacggaggggttt

SEQ ID NO: 99 - DNA, 804 bp, synthetic construct
  source 1..804: mol_type = other DNA
gcgaattaatacgactcactatagggcttaagtataaggaggaaaaaatatggtgtctaagggagaagaactgtttaccggggtagtgcccattcttgtcgagcttgacggtgacgtaaatggccataagttctcagtttctggagagggcgagggagacgctacgtacgggaagttgacactgaaattcatttgcacgacgggtaaattgcctgtgccttggcccaccttagtcacaacattaacgtacggggtgcagtgcttcagtcgctacccagatcacatgaagcaacacgacttcttcaaatcagccatgcctgaaggatacgtgcaggaacgtacaatcttcttcaaggatgacggcaattacaagacccgtgcggaagttaaatttgagggcgacacacttgtgaatcgcatcgaattgaaaggtattgatttcaaagaagatgggaatatccttgggcataagttggaatataactacaacagtcacaatgtttacatcatggcagataagcagaaaaacggtattaaggtaaatttcaaaattcgccataacattgaggatggttcggttcaacttgccgatcactaccagcaaaacacgcctattggggatggaccagttcttctgcccgataatcattacctgagcacgcaatcggctttgtcaaaagacccaaacgaaaaacgtgaccatatggtcttgcttgagttcgtaacggccgccggtattacgcttggtatggacgaattatataagtgataactagcataacccctctctaaacggaggggttt

SEQ ID NO: 100 - DNA, 564 bp, synthetic construct
  source 1..564: mol_type = other DNA
gcgaattaatacgactcactatagggcttaagtataaggaggaaaaaatatgatcagtctgattgcggcgttagcggtagatcgcgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgtgggtgaagtcggtggatgaagccatcgcggcgtgtggtgacgtaccagaaatcatggtgattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcggtaataactagcataacccctctctaaacggaggggttt

SEQ ID NO: 101 - DNA, 3281 bp, synthetic construct
  source 1..3281: mol_type = other DNA
cccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatcgacactgctcgatccgctcgcaccactagttctagaggtacccctgcaggttggaccctcgtacagaagctaatacgactcactatagggaaataagagagaaaagaagagtaagaagaaatataagagaattcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatccgctgccttctgcggggcttgccttctggccatgcccttcttctctcccttgcacctgtacctcttggtctttgaataaagcctgagtaggaagaatattaaaaaaaaaaaaaaaaaaaaaaaaaaaaaaaaaaaaaaaaaaaaattaattaacatatggcggccgctctagaactagtgtcgacgcggatcgacgagagcagcgcgactggatcagttctggacgagcgagctgtcgtccgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttcttgaggacctaaatgtaatcacctggctcaccttcgggtgggcctttcttgaggacctaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 102 - DNA, 1095 bp, synthetic construct
  source 1..1095: mol_type = other DNA
tgtaaaacgacggccagttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatggtttccaaaggagaagagttgtttactggggtggtcccaattcttgtggaattggatggcgacgttaatgggcataaattcagcgtgagtggtgaaggtgaaggagacgccacttacggcaagctaaccctaaaatttatatgtacgactggtaaacttcctgtaccatggcctactcttgtaactacattaacgtatggagtacaatgcttttctaggtatccggatcatatgaagcagcatgactttttcaagtctgcgatgccggaagggtatgtgcaggaaaggactattttcttcaaagacgatggtaactataagactagggccgaggtcaagtttgagggggatacgcttgtgaacaggatcgagctgaaaggtatcgattttaaggaggatgggaacattcttgggcacaagctagagtataattataattcccacaatgtttatataatggccgataagcaaaaaaatggcattaaggtgaatttcaagattcgtcataatattgaagacggttcagtgcaactagcagaccactatcagcaaaacacacctataggagatggtcctgtgttactacccgacaatcactacttgtccacccagtctgctctatccaaagatcctaacgagaaacgtgaccatatggtcttgttagagttcgttacagcggcaggcataaccttaggtatggacgagttatacaaataatatataactgtctagaaataaagagtatcatctttcaaaggtcatagctgtttcctg

SEQ ID NO: 103 - DNA, 3391 bp, synthetic construct
  source 1..3391: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaaatgcggtgtgaaataccgcacagatgcgtaaggagaaaataccgcatcaggcgccattcgccattcaggctgcgcaactgttgggaagggcgatcggtgcgggcctcatcgctattacgccagctggcgaaagggggatgtgctgcaaggcgattaagttgggtaacgccagggttttcccagtcacgacgttgtaaaacgacggccagtgcaacgcgatgacgatggatagcgattcatcgatgagctgacccgatcgccgccgccggagggttgcgtttgagacgggcgacagatccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactagggtgggcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctccatcagttctggaccagcgagctgtgctgcgactcgtggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgtcgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactctacctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 104 - DNA, 2225 bp, synthetic construct
  source 1..2225: mol_type = other DNA
cccgtgtaaaacgacggccagtttatctagtcagcttgattctagctgatcgtggaccggaaggtgagccagtgagttgattgcagtccagttacgctggagtctgaggctcgtcctgaatgatatgcggcctcgtcaagaaagcagagactgccgggagcaaagagaatagactgtagggaatactaagcatagactccagggtaaacaaagcatagactgaggggaagaccaaggatagactgctgggaacataaagaatagaatgatgggacaaagaggaagagagacggggacacacacacttagaatctgtgggagtctaagcagaagaagaagaggaacacaatgcatagattgccgggcgcgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttcttgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 105 - DNA, 10 bp, synthetic construct
  source 1..10: mol_type = other DNA
gcattaatgc

SEQ ID NO: 106 - DNA, 53 bp, synthetic construct
  source 1..53: mol_type = other DNA
tggtgaaccgcatcgagctgaagggcatcgacttcaaggaggacggcaacatc

SEQ ID NO: 107 - RNA, 96 nt, synthetic construct
  source 1..96: mol_type = other RNA
gctgaagggcatcgacttcagttttagagctagaaatagcaagttaaaataaggctagtccgttatcaacttgaaaaagtggcaccgagtcggtgc

SEQ ID NO: 108 - DNA, 20 bp, synthetic construct
  source 1..20: mol_type = other DNA
  modified_base 5: mod_base = OTHER; note = 2-aminoadenine
  modified_base 6: mod_base = OTHER; note = 2-aminoadenine
  modified_base 11: mod_base = OTHER; note = 2-aminoadenine
  modified_base 15: mod_base = OTHER; note = 2-aminoadenine
  modified_base 20: mod_base = OTHER; note = 2-aminoadenine
gctgnngggcntcgncttcn

SEQ ID NO: 109 - DNA, 23 bp, synthetic construct
  source 1..23: mol_type = other DNA
gctgaagggcatcgacttcaagg

SEQ ID NO: 110 - RNA, 20 nt, synthetic construct
  source 1..20: mol_type = other RNA
gctgaagggcatcgacttca

SEQ ID NO: 111 - DNA, 20 bp, synthetic construct
  source 1..20: mol_type = other DNA
tgaagtcgatgcccttcagc

SEQ ID NO: 112 - DNA, 20 bp, synthetic construct
  source 1..20: mol_type = other DNA
  modified_base 3: mod_base = OTHER; note = 2-aminoadenine
  modified_base 12: mod_base = OTHER; note = 2-aminoadenine
  modified_base 17: mod_base = OTHER; note = 2-aminoadenine
  modified_base 18: mod_base = OTHER; note = 2-aminoadenine
tgnngtcgntgcccttcngc

SEQ ID NO: 113 - RNA, 39 nt, synthetic construct
  source 1..39: mol_type = other RNA
aatttctactgttgtagatcgtcgccgtccagctcgacc

SEQ ID NO: 114 - DNA, 42 bp, synthetic construct
  source 1..42: mol_type = other DNA
tggtgcccatcctggtcgagctggacggcgacgtaaacggcc

SEQ ID NO: 115 - DNA, 578 bp, synthetic construct
  source 1..578: mol_type = other DNA
gatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacac

SEQ ID NO: 116 - DNA, 72 bp, synthetic construct
  source 1..72: mol_type = other DNA
gcgaattaatacgactcactatagggcttaagtataaggaggaaaaaatatggtgtctaagggagaagaact

SEQ ID NO: 117 - DNA, 70 bp, synthetic construct
  source 1..70: mol_type = other DNA
gaagaaatataagagaattcgccgccaccatgcaccaccaccaccaccacgtgagcaagggcgaggagct

SEQ ID NO: 118 - DNA, 39 bp, synthetic construct
  source 1..39: mol_type = other DNA
gcagaaggcagcggatccttacttgtacagctcgtccat

SEQ ID NO: 119 - RNA, 39 nt, synthetic construct
  source 1..39: mol_type = other RNA
  modified_base 1: mod_base = OTHER; note = 2-aminoadenine
  modified_base 2: mod_base = OTHER; note = 2-aminoadenine
  modified_base 8: mod_base = OTHER; note = 2-aminoadenine
  modified_base 16: mod_base = OTHER; note = 2-aminoadenine
  modified_base 18: mod_base = OTHER; note = 2-aminoadenine
  modified_base 31: mod_base = OTHER; note = 2-aminoadenine
  modified_base 37: mod_base = OTHER; note = 2-aminoadenine
nntttctnctgttgtngntcgtcgccgtccngctcgncc

SEQ ID NO: 120 - DNA, 34 bp, synthetic construct
  source 1..34: mol_type = other DNA
atcctggtcgagctggacggcgacgtaaacggcc

SEQ ID NO: 121 - DNA, 29 bp, synthetic construct
  source 1..29: mol_type = other DNA
tttacgtcgccgtccagctcgaccaggat

SEQ ID NO: 122 - DNA, 27 bp, synthetic construct
  source 1..27: mol_type = other DNA
tttacgtcgccgtccagctcgaccaga

SEQ ID NO: 123 - DNA, 20 bp, synthetic construct
  source 1..20: mol_type = other DNA
tttaccttcttcttgatatg

SEQ ID NO: 124 - DNA, 20 bp, synthetic construct
  source 1..20: mol_type = other DNA
  modified_base 4: mod_base = OTHER; note = 2-aminoadenine
  modified_base 16: mod_base = OTHER; note = 2-aminoadenine
  modified_base 18: mod_base = OTHER; note = 2-aminoadenine
tttnccttcttcttgntntg

SEQ ID NO: 125 - DNA, 20 bp, synthetic construct
  source 1..20: mol_type = other DNA
  modified_base 1: mod_base = OTHER; note = 2-aminoadenine
  modified_base 2: mod_base = OTHER; note = 2-aminoadenine
  modified_base 7: mod_base = OTHER; note = 2-aminoadenine
  modified_base 8: mod_base = OTHER; note = 2-aminoadenine
  modified_base 9: mod_base = OTHER; note = 2-aminoadenine
  modified_base 19: mod_base = OTHER; note = 2-aminoadenine
nnggccnnncctcggcttnc

SEQ ID NO: 126 - DNA, 20 bp, synthetic construct
  source 1..20: mol_type = other DNA
cactaacacgcatatttgat

SEQ ID NO: 127 - DNA, 20 bp, synthetic construct
  source 1..20: mol_type = other DNA
  modified_base 2: mod_base = OTHER; note = 2-aminoadenine
  modified_base 5: mod_base = OTHER; note = 2-aminoadenine
  modified_base 6: mod_base = OTHER; note = 2-aminoadenine
  modified_base 8: mod_base = OTHER; note = 2-aminoadenine
  modified_base 12: mod_base = OTHER; note = 2-aminoadenine
  modified_base 14: mod_base = OTHER; note = 2-aminoadenine
  modified_base 19: mod_base = OTHER; note = 2-aminoadenine
cnctnncncgcntntttgnt

SEQ ID NO: 128 - DNA, 20 bp, synthetic construct
  source 1..20: mol_type = other DNA
ttcatatgttcttctagctt

SEQ ID NO: 129 - DNA, 20 bp, synthetic construct
  source 1..20: mol_type = other DNA
ctgaatatctatgcattaat

SEQ ID NO: 130 - DNA, 34 bp, synthetic construct
  source 1..34: mol_type = other DNA
gggcctgagtccgagcagaagaagaagggtccca

SEQ ID NO: 131 - RNA, 42 nt, synthetic construct
  source 1..42: mol_type = other RNA
  modified_base 2: mod_base = OTHER; note = 2-aminoadenine
  modified_base 8: mod_base = OTHER; note = 2-aminoadenine
  modified_base 11: mod_base = OTHER; note = 2-aminoadenine
  modified_base 13: mod_base = OTHER; note = 2-aminoadenine
  modified_base 14: mod_base = OTHER; note = 2-aminoadenine
  modified_base 16: mod_base = OTHER; note = 2-aminoadenine
  modified_base 17: mod_base = OTHER; note = 2-aminoadenine
  modified_base 19: mod_base = OTHER; note = 2-aminoadenine
  modified_base 20: mod_base = OTHER; note = 2-aminoadenine
  modified_base 26: mod_base = OTHER; note = 2-aminoadenine
  modified_base 28: mod_base = OTHER; note = 2-aminoadenine
  modified_base 32: mod_base = OTHER; note = 2-aminoadenine
gngtccgngcngnngnngnngttttngngctntgctgttttg

SEQ ID NO: 132 - RNA, 86 nt, synthetic construct
  source 1..86: mol_type = other RNA
ggaaccattcaaaacagcatagcaagttaaaataaggctagtccgttatcaacttgaaaaagtggcaccgagtcggtgcttttttt

SEQ ID NO: 133 - RNA, 96 nt, synthetic construct
  source 1..96: mol_type = other RNA
gagtccgagcagaagaagaagttttagagctagaaatagcaagttaaaataaggctagtccgttatcaacttgaaaaagtggcaccgagtcggtgc

SEQ ID NO: 134 - DNA, 20 bp, synthetic construct
  source 1..20: mol_type = other DNA
  modified_base 2: mod_base = OTHER; note = 2-aminoadenine
  modified_base 8: mod_base = OTHER; note = 2-aminoadenine
  modified_base 11: mod_base = OTHER; note = 2-aminoadenine
  modified_base 13: mod_base = OTHER; note = 2-aminoadenine
  modified_base 14: mod_base = OTHER; note = 2-aminoadenine
  modified_base 16: mod_base = OTHER; note = 2-aminoadenine
  modified_base 17: mod_base = OTHER; note = 2-aminoadenine
  modified_base 19: mod_base = OTHER; note = 2-aminoadenine
  modified_base 20: mod_base = OTHER; note = 2-aminoadenine
gngtccgngcngnngnngnn

SEQ ID NO: 135 - DNA, 23 bp, synthetic construct
  source 1..23: mol_type = other DNA
gagtccgagcagaagaagaaggg

SEQ ID NO: 136 - RNA, 20 nt, synthetic construct
  source 1..20: mol_type = other RNA
gagtccgagcagaagaagaa

SEQ ID NO: 137 - DNA, 20 bp, synthetic construct
  source 1..20: mol_type = other DNA
  modified_base 17: mod_base = OTHER; note = 2-aminoadenine
ttcttcttctgctcggnctc

SEQ ID NO: 138 - DNA, 20 bp, synthetic construct
  source 1..20: mol_type = other DNA
aataatttctattatattac

SEQ ID NO: 139 - DNA, 20 bp, synthetic construct
  source 1..20: mol_type = other DNA
  modified_base 1: mod_base = OTHER; note = 2-aminoadenine
  modified_base 2: mod_base = OTHER; note = 2-aminoadenine
  modified_base 4: mod_base = OTHER; note = 2-aminoadenine
  modified_base 5: mod_base = OTHER; note = 2-aminoadenine
  modified_base 11: mod_base = OTHER; note = 2-aminoadenine
  modified_base 14: mod_base = OTHER; note = 2-aminoadenine
  modified_base 16: mod_base = OTHER; note = 2-aminoadenine
  modified_base 19: mod_base = OTHER; note = 2-aminoadenine
nntnntttctnttntnttnc

SEQ ID NO: 140 - DNA, 20 bp, synthetic construct
  source 1..20: mol_type = other DNA
gagtgacactgtgaaggaga

SEQ ID NO: 141 - DNA, 20 bp, synthetic construct
  source 1..20: mol_type = other DNA
  modified_base 2: mod_base = OTHER; note = 2-aminoadenine
  modified_base 6: mod_base = OTHER; note = 2-aminoadenine
  modified_base 8: mod_base = OTHER; note = 2-aminoadenine
  modified_base 14: mod_base = OTHER; note = 2-aminoadenine
  modified_base 15: mod_base = OTHER; note = 2-aminoadenine
  modified_base 18: mod_base = OTHER; note = 2-aminoadenine
  modified_base 20: mod_base = OTHER; note = 2-aminoadenine
gngtgncnctgtgnnggngn

SEQ ID NO: 142 - DNA, 20 bp, synthetic construct
  source 1..20: mol_type = other DNA
gaagaccaaggatagactgc

SEQ ID NO: 143 - DNA, 20 bp, synthetic construct
  source 1..20: mol_type = other DNA
  modified_base 2: mod_base = OTHER; note = 2-aminoadenine
  modified_base 3: mod_base = OTHER; note = 2-aminoadenine
  modified_base 5: mod_base = OTHER; note = 2-aminoadenine
  modified_base 8: mod_base = OTHER; note = 2-aminoadenine
  modified_base 9: mod_base = OTHER; note = 2-aminoadenine
  modified_base 12: mod_base = OTHER; note = 2-aminoadenine
  modified_base 14: mod_base = OTHER; note = 2-aminoadenine
  modified_base 16: mod_base = OTHER; note = 2-aminoadenine
gnngnccnnggntngnctgc

SEQ ID NO: 144 - DNA, 20 bp, synthetic construct
  source 1..20: mol_type = other DNA
gaacataaagaatagaatga

SEQ ID NO: 145 - DNA, 23 bp, synthetic construct
  source 1..23: mol_type = other DNA
gaacacaaagcatagactgcggg

SEQ ID NO: 146 - DNA, 23 bp, synthetic construct
  source 1..23: mol_type = other DNA
gaacataaagaatagaatgatgg

SEQ ID NO: 147 - DNA, 23 bp, synthetic construct
  source 1..23: mol_type = other DNA
gaagaccaaggatagactgctgg

SEQ ID NO: 148 - DNA, 23 bp, synthetic construct
  source 1..23: mol_type = other DNA
gtaaacaaagcatagactgaggg

SEQ ID NO: 149 - DNA, 23 bp, synthetic construct
  source 1..23: mol_type = other DNA
gtcaagaaagcagagactgccgg

SEQ ID NO: 150 - DNA, 23 bp, synthetic construct
  source 1..23: mol_type = other DNA
gaggccgagcagaagaaagacgg

SEQ ID NO: 151 - DNA, 23 bp, synthetic construct
  source 1..23: mol_type = other DNA
gagtctaagcagaagaagaagag

SEQ ID NO: 152 - DNA, 23 bp, synthetic construct
  source 1..23: mol_type = other DNA
aaacataaagcatagactgcaaa

SEQ ID NO: 153 - DNA, 23 bp, synthetic construct
  source 1..23: mol_type = other DNA
gaacacaatgcatagattgccgg

SEQ ID NO: 154 - DNA, 822 bp, synthetic construct
  source 1..822: mol_type = other DNA
gcgaattaatacgactcactatagggcttaagtataaggaggaaaaaatcaccaccaccaccaccacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataactagcataacccctctctaaacggaggggttt

SEQ ID NO: 155 - DNA, 193 bp, synthetic construct
  source 1..193: mol_type = other DNA
acactctttccctacacgacgctcttccgatctggaggacaaagtacaaacggcagaagctggaggaggaagggcctgagtccgagcagaagaagaagggctcccatcacatcaaccggtggcgcattgccacgaagcaggccaatggggaggacatcgatagatcggaagagcacacgtctgaactccagtc

SEQ ID NO: 156 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 157 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 158 - DNA, 1087 bp, synthetic construct
  source 1..1087: mol_type = other DNA
aattaatagaagtcaatgtagggaaggaaatatggcagaaattaaacatatattcataattcaaaagtgaaatatggtagtgaaaacatatgtctgccatatatataatatatgcatatgtaatacatgtgtatatatgtatgctgtgagctaaatcatatctacaacttaaaaaatgaacataatggaataaggatattttaaattattatgttaaaatgttaagtatacttggagttggtgaaaaaaaatcaattcatttatacagacaatccaagaaggtatgacacattactgttttcataggaaaaataggcaagttgcaatcctttgaaatagatttggcttttgatatcatcaatatctttgaaggactctgggtaaaatatctgtttcccatcacattttccaattaattgctatgtgaaaaaatatagtttaaaggccaaacctcggcttacctgaaattttcgaagtttattcatatgttcttctagcttttggcagctttccaccaactgggaggaaagtttcttccagtgcccctcaatctcttcaaattctgacagatatttctggcatatttctgaaggtgctttcttggccatctccttcacagtgtcactcagatagttgaagccattttgttgctctttcaaagaactttgcagagcctcaaaattaaatagaagttcatttacactaacacgcatatttgatgagtttcattcatatcaagaagaaggtaaataaaagtttatgtaatttagagatctaaacaaagtagaatatctgataaagcaatatatatgtattcttatgaataatttctattatattacagggcatattatatttagacagaaatttagaagtcataattttactgaatatctatgcattaataactatgtttattatatatgtgatacattttaacttacctacagtgaaacattattaagacatttaggcaatatatgattaatatattttcagatttaaaaagaataagtatacaacatgggatttttagaatcaacaaaaaaattagtctttatatgtcctcacatcacagaagtttctc

SEQ ID NO: 159 - DNA, 20 bp, synthetic construct
  source 1..20: mol_type = other DNA
gaatcaacaaaaaaattagt

SEQ ID NO: 160 - DNA, 20 bp, synthetic construct
  source 1..20: mol_type = other DNA
agatttaaaaagaataagta

SEQ ID NO: 161 - DNA, 20 bp, synthetic construct
  source 1..20: mol_type = other DNA
aaagagcaacaaaatggctt

SEQ ID NO: 162 - DNA, 23 bp, synthetic construct
  source 1..23: mol_type = other DNA
tggttgcccaccctagtcattgg

SEQ ID NO: 163 - DNA, 20 bp, synthetic construct
  source 1..20: mol_type = other DNA
  modified_base 5: mod_base = OTHER; note = 2-aminoadenine
  modified_base 6: mod_base = OTHER; note = 2-aminoadenine
  modified_base 11: mod_base = OTHER; note = 2-aminoadenine
  modified_base 15: mod_base = OTHER; note = 2-aminoadenine
  modified_base 20: mod_base = OTHER; note = 2-aminoadenine
gctgnngggcntcgncttcn

SEQ ID NO: 164 - DNA, 23 bp, synthetic construct
  source 1..23: mol_type = other DNA
gctgaagggcatcgacttcaagg

SEQ ID NO: 165 - DNA, 473 bp, synthetic construct
  source 1..473: mol_type = other DNA
ggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagat

(19) United States
(12) Patent Application Publication
(10) Pub. No.: US 2026/0218171 A1
(43) Pub. Date: Jul. 30, 2026

(54) EXPANDING APPLICATIONS OF ZGTC ALPHABET IN PROTEIN EXPRESSION AND GENE EDITING

(71) Applicant: Trustees of Tufts College, Medford, MA (US)

(72) Inventors: Shuliang Gao, Medford, MA (US); Qiaobing Xu, Lexington, MA (US)

(21) Appl. No.: 19/141,883

(22) PCT Filed: Dec. 22, 2023
(86) PCT No.: PCT/US23/85701

§ 371 Date: Jun. 20, 2025

Related U.S. Application Data
(60) Provisional application No. 63/434,647, filed on Dec. 22, 2022.

(51) Int. Cl. C12N 15/11; C12N 9/22; C12N 15/90

(52) U.S. Cl. CPC C12N 15/11; C12N 9/226; C12N 15/907; C12N 2310/20; C12N 2310/333

(57) ABSTRACT

Disclosed are nucleic acids comprising a 2-aminoadenine (Z) base, and their uses in protein expression and gene editing. Standard ATGC-DNA (A-DNA) and AUGC-RNA (A-RNA) are composed of four standard nucleotides, each with a different nucleobase: adenine (A), thymine (T)/Uridine (U), guanine (G), and cytosine (C). These nucleobases form the genetic alphabet, ATGC, which make up the base pairs that follow Watson-Crick base-pairing rules, which are conserved across almost all domains of life. Why nature has evolved to primarily use these two base pair combinations remains one of the greatest mysteries in science.

### RELATED APPLICATION

This application claims the benefit of priority to U.S. Provisional Patent Application No. 63/434,647, filed on Dec. 22, 2022; the entire contents of which are hereby incorporated by reference.

### GOVERNMENT SUPPORT

This invention was made with government support under grant TR002636 awarded by the National Institutes of Health. The government has certain rights in the invention.

### BACKGROUND OF THE INVENTION

Standard ATGC-DNA (A-DNA) and AUGC-RNA (A-RNA) are composed of four standard nucleotides, each with a different nucleobase: adenine (A), thymine (T)/Uridine (U), guanine (G), and cytosine (C). These nucleobases form the genetic alphabet, ATGC, which make up the base pairs that follow Watson-Crick base-pairing rules, which are conserved across almost all domains of life. Why nature has evolved to primarily use these two base pair combinations remains one of the greatest mysteries in science. Researchers have described the distribution and evolution of non-conventional nucleobases, such as the Z-base, in biological systems (1-5). The Z base, also known as 2-aminoadenine, was first discovered in the S-2L cyanophage genome, forming the ZTGC genetic alphabet that violates conventional Watson-Crick base pairing rules (1, 6). Phages and viruses carrying the ZTGC-DNA (Z-DNA) genome are widely spread on earth (2). The natural synthetic pathway of the Z-base and Z-DNA polymerases have been identified (3-5, 7, 8). These findings encourage the use of synthetic biology approaches to develop and manufacture phages or viruses with artificially designed Z-DNA genomes.

Compared to the standard A-base, the Z-base has an extra amino group on the 2 position that allows it to form a third hydrogen bond with a T-base in strands of DNA (9). When a Z-T pair is introduced in synthetic oligonucleotides, the extra hydrogen bond enhances the thermal stability, sequence specificity, and Type II restriction endonuclease (RE) resistance properties (2, 10, 11). Furthermore, in vitro tube assays have showed that Z-bases can be accepted as potential substrates for several standard RNA and DNA polymerases (12-14). In the field of disease therapeutics Z-DNA is predicted to have many advantages over A-DNA, including with nucleic acid drugs (5). In recent years, nucleic acid therapies have achieved significant success. Lipid nanoparticles (LNPs) can deliver DNA or RNA payloads synthesized by chemical or in vitro transcription (IVT) to achieve in vitro and in vivo gene regulation or editing (15). Replacing certain standard nucleotides with modified nucleotides can improve the performance and efficacy of nucleic acid cargoes (16, 17). However, it is considered that these processes all depend on Watson-Crick pairing for binding and targeting. The discovery of the Z-base synthesis pathway further enriches the biodiversity of natural bases. Exploring and evaluating the compatibility of the Z-base in complex biological systems can help us learn about non-Watson-Crick pairing principles present in viruses, develop more potential applications for the ZTGC alphabet, and further contribute to the optimization of nucleic acid drugs. However, many questions regarding the functionality of Z-DNA or ZUGC-RNA (Z-RNA) have not yet been explored. Currently, it is not known whether Z-DNA or Z-RNA constructs are compatible with most of the cellular machinery and enzymes (2, 18).

### SUMMARY OF THE INVENTION

The present disclosure is based, at least in part, on the discovery that (1) Z-DNA and Z-RNA are compatible in various living systems, including bacteria, yeast, and mammalian cells; and (2) RNA-guided endonucleases including Cas9 and Cas12a utilize Z-RNA through non-Watson-Crick base pairing processes to mediate efficient DNA cleavage and achieve precise gene editing in mammalian cells.

In some aspects, provided herein is a nucleic acid comprising less than or equal to 2500 (e.g., less than or equal to 2000) nucleotides, wherein at least 15% of said nucleotides comprise a 2-aminoadenine (Z) base.

In some embodiments, at least 38% or at least 39% of said nucleotides comprise a Z base. In some embodiments, the nucleic acid does not comprise an adenine (A) base. In some embodiments, the nucleic acid comprises at least 100 or at least 160 nucleotides. In some embodiments, the nucleic acid is a DNA comprising at least one intron, a cDNA, or an mRNA. In some embodiments, the nucleic acid is an mRNA comprising a poly(Z) tail. In some embodiments, the method further comprises at least one chemical modification.

In some aspects, provided herein is a vector comprising a nucleic acid described herein. In some embodiments, the vector is an expression vector.

In some aspects, provided herein is a method of expressing a protein, comprising contacting a cell with a nucleic acid described herein, or with a vector described herein. In some embodiments, the cell is a prokaryotic cell or a eukaryotic cell.

In some aspects, provided herein is a composition or kit, comprising: (a) a Cas12a RNA-guided endonuclease or a nucleic acid encoding the Cas12a RNA-guided endonuclease; and (b) a Cas12a crRNA comprising at least one Z base.

In some aspects, provided herein is a method of cleaving or modifying a target DNA, comprising: contacting the target DNA or a cell comprising the target DNA with: (a) a Cas12a RNA-guided endonuclease, or a nucleic acid encoding the Cas12a RNA-guided endonuclease; and (b) a Cas12a crRNA comprising at least one Z base, wherein the Cas12a RNA-guided endonuclease and the Cas12a crRNA form a complex that cleaves or modifies the target DNA. In some embodiments, the target DNA is a plasmid DNA.

In some embodiments, the Cas12a crRNA comprises at least 7 Z bases. In some embodiments, the Cas12a crRNA does not comprise an A base. In some embodiments, the Cas12a RNA-guided endonuclease is LbCas12a (LbCpf1).

In some embodiments, the Cas12a crRNA comprising at least one Z base induces higher cleavage of the target DNA compared to the corresponding Cas12a crRNA where the at least one Z base is substituted with A base. In some embodiments, the Cas12a crRNA comprising at least one Z base induces at least 1.1-fold (e.g., 1.2-fold, 1.4-fold, 1.8-fold, 3.3-fold, or 6-fold) cleavage of the target DNA compared to the corresponding Cas12a crRNA where the at least one Z base is substituted with A base.

In some embodiments, the Cas12a crRNA comprises at least one Z base in the seed region. In some embodiments, the Cas12a crRNA comprises a 20-nt guide sequence complimentary to a target DNA site with an A or Z content from 15% to 35%. In some embodiments, the Cas12a crRNA comprises a 20-nt guide sequence complimentary to a target DNA site with an AT or ZT content from 45% to 75%, or from 45% to 85%.

In some embodiments, the target DNA comprises a 5′-TTTA, 5′-TTTC, or 5′-TTTG PAM motif. In some embodiments, the target DNA comprises a T-base at position 5′-PAM-6, 8, 10, 18, 19-3′. In some embodiments, the target DNA comprises at least one Z base.

In some aspects, provided herein is a method of improving cleavage activity or editing efficiency of a complex comprising a Cas12a RNA-guided endonuclease and a Cas12a crRNA, comprising substituting at least one A base of the Cas12a crRNA with a Z base. In some embodiments, the method comprises substituting all A bases of the Cas12a crRNA with Z bases. In some embodiments, the method comprises substituting at least one A base of the DNA substrate with a Z base. In some embodiments, the method comprises substituting all A bases of the DNA substrate with Z bases.

In some aspects, provided herein is a method of cleaving or modifying a target DNA comprising at least one Z base, comprising: contacting the target DNA or a cell comprising the target DNA with: (a) a Cas12a RNA-guided endonuclease or a nucleic acid encoding the Cas12a RNA-guided endonuclease; and (b) a Cas12a crRNA or a nucleic acid encoding a Cas12a crRNA, wherein the Cas12a RNA-guided endonuclease and the Cas12a crRNA form a complex that cleaves or modifies the target DNA comprising at least one Z base.

In some aspects, provided herein is a composition or kit, comprising: (a) a Cas9 protein or a nucleic acid encoding the Cas9 protein; (b) a Cas9 crRNA comprising at least one Z base; and (c) a Cas9 tracrRNA.

In some aspects, provided herein is a method of cleaving or modifying a target DNA, comprising: contacting the target DNA or a cell comprising the target DNA with: (a) a Cas9 protein, or a nucleic acid encoding the Cas9 protein; (b) a Cas9 crRNA comprising at least one Z base, and (c) a Cas9 tracrRNA; wherein the Cas9 protein, the Cas9 crRNA, and the Cas9 tracrRNA form a complex that cleaves or modifies the target DNA. In some embodiments, the target DNA is a plasmid DNA. In some embodiments, the target DNA comprises at least one Z base.

In some embodiments, the Cas9 crRNA comprises at least 12 Z bases. In some embodiments, the Cas9 crRNA does not comprise an A base. In some embodiments, the Cas9 crRNA comprises a 20-nt guide sequence complimentary to a target DNA site with an A or Z content from 35% to 60%. In some embodiments, the Cas9 crRNA comprises a 20-nt guide sequence complimentary to a target DNA site with an AT or ZT content from 45% to 90%. In some embodiments, Cas9 tracrRNA comprises at least one Z base. In some embodiments, the Cas9 tracrRNA does not comprise an A base.

In some aspects, provided herein is a method of cleaving or modifying a target DNA comprising at least one Z base, comprising: contacting the target DNA with: (a) a Cas9 protein or a nucleic acid encoding the Cas9 protein; and (b) a Cas9 guide RNA (gRNA) or a nucleic acid encoding a Cas9 gRNA, wherein the Cas9 protein and the Cas9 gRNA form a complex that cleaves or modifies the target DNA comprising at least one Z base. In some embodiments, the target DNA comprises a Z base in a spacer region or a PAM region or both. In some embodiments, the target DNA is a plasmid DNA.

In some aspects, provided herein is a method of modifying a target DNA comprising: contacting the target DNA or a cell comprising the target DNA with: (a) a Cas9-guided base editor or a nucleic acid encoding the Cas9-guided base editor; (b) a Cas9 crRNA comprising at least one Z base; and (c) a Cas9 tracrRNA, wherein the Cas9-guided base editor, the Cas9 crRNA, and the Cas9 tracrRNA form a complex that induces a base change of the target DNA.

In some embodiments, the Cas9-guided base editor is an adenine base editor (ABE). In some embodiments, the Cas9-guided base editor is ABE8e. In some embodiments, the Cas9-guided base editor, the Cas9 crRNA, and the Cas9 tracrRNA form a complex that induces a A-to-G change of the target DNA. In some embodiments, the Cas9-guided base editor, the Cas9 crRNA, and the Cas9 tracrRNA form a complex that induces A-to-G changes with a frequency of at least 5%. In some embodiments, the Cas9 crRNA comprises at least 8 Z bases. In some embodiments, the Cas9 crRNA does not comprise an A base. In some embodiments, the cell is a mammalian cell.

In some aspects, provided herein is a nucleic acid comprising less than or equal to 2,000 nucleotides, wherein at least 15% of said nucleotides comprise a 2-aminoadenine (Z) base.

In some embodiments, at least 39% of said nucleotides comprise a Z base. In some embodiments, the nucleic acid does not comprise an adenine (A) base. In some embodiments, the nucleic acid comprises at least 160 nucleotides. In some embodiments, the nucleic acid is a DNA comprising at least one intron, a cDNA, or an mRNA. In some embodiments, the nucleic acid is an mRNA comprising a poly(Z) tail. In some embodiments, the method further comprises at least one chemical modification.

In some aspects, provided herein is a vector comprising a nucleic acid described herein. In some embodiments, the vector is an expression vector.

In some aspects, provided herein is a method of expressing a protein, comprising contacting a cell with a nucleic acid described herein, or with a vector described herein. In some embodiments, the cell is a prokaryotic cell or a eukaryotic cell.

In some aspects, provided herein is a composition, comprising: (a) a CRISPR from Prevotella and Francisella 1 (Cpf1) RNA-guided nuclease or a nucleic acid encoding the Cpf1 RNA-guided nuclease; and (b) a Cpf1 guide RNA (gRNA) comprising at least one Z base.

In some embodiments, the Cpf1 gRNA comprises at least 7 Z bases. In some embodiments, the Cpf1 gRNA does not comprise an A base. In some embodiments, the Cpf1 RNA-guided nuclease is LbCpf1.

In some aspects, provided herein is a method of cleaving a target DNA, comprising: contacting the target DNA with: (a) a Cpf1 RNA-guided nuclease or a nucleic acid encoding the Cpf1 RNA-guided nuclease; and (b) a Cpf1 guide RNA (gRNA) comprising at least one Z base, wherein the Cpf1 RNA-guided nuclease and the Cpf1 gRNA form a complex that cleaves the target DNA.

In some embodiments, the Cpf1 gRNA comprises at least 7 Z bases. In some embodiments, the Cpf1 gRNA does not comprise an A base. In some embodiments, the Cpf1 RNA-guided nuclease is LbCpf1. In some embodiments, the target DNA is a plasmid DNA. In some embodiments, the Cpf1 gRNA comprising at least one Z base induces higher cleavage of the target DNA compared to the corresponding Cpf1 gRNA where the at least one Z base is substituted with A base. In some embodiments, the Cpf1 gRNA comprising at least one Z base induces at least 1.1-fold cleavage of the target DNA compared to the corresponding Cpf1 gRNA where the at least one Z base is substituted with A base.

In some aspects, provided herein is a method of improving cleavage activity of a complex comprising a Cpf1 RNA-guided nuclease and a Cpf1 guide RNA (gRNA), comprising substituting at least one A base of the Cpf1 gRNA with Z base. In some embodiments, the method comprises substituting all A bases of the Cpf1 gRNA with Z bases.

In some aspects, provided herein is a method of cleaving a target DNA comprising at least one Z base, comprising: contacting the target DNA with: (a) a Cas9 protein or a nucleic acid encoding the Cas9 protein; and (b) a Cas9 guide RNA (gRNA) or a nucleic acid encoding a Cas9 gRNA, wherein the Cas9 protein and the Cas9 gRNA form a complex that cleaves the target DNA comprising at least one Z base.

In some embodiments, the target DNA comprises a Z base in a spacer region or a PAM region or both. In some embodiments, the target DNA is a plasmid DNA.

In some aspects, provided herein is a kit, comprising: (a) a Cpf1 RNA-guided nuclease or a nucleic acid encoding the Cpf1 RNA-guided nuclease; and (b) a Cpf1 guide RNA (gRNA) comprising at least one Z base.

In some embodiments, the Cpf1 gRNA comprises at least 7 Z bases. In some embodiments, the Cpf1 gRNA does not comprise an A base. In some embodiments, the Cpf1 RNA-guided nuclease is LbCpf1.

### BRIEF DESCRIPTION OF THE DRAWINGS

FIG. 1A is a Schematic representation of DNA amplicons in this investigation. Amplicons contains 5′ and 3′ UTR sequences.

FIG. 1B shows PCR amplification yield using different dNTPs. Taq DNA polymerase was used in this test. dATP group: substrates consist of dATP, dTTP, dGTP, dCTP; dZTP group: substrates consist of dZTP, dTTP, dGTP and dCTP. n=3.

FIG. 1C shows Melting curve of DNA containing dATP or dZTP.

FIG. 1D shows A:T content in sticky ends of restriction endonucleases used for DNA cleavage. BsrF I, 5′- [symbol] CCGGY-3′; FauI, 5′-CCCGC(GCC [symbol] AGG-3′; BstYI, 5′- [symbol] GATCY-3′; BsrI, 5′-ACTGGG [symbol] -3′; EcoRI, 5′- [symbol] AATTC-3′.

FIG. 1E shows In vitro DAN cleavage assay of restriction enzyme digestions of the DNA. BsrF I, 37° C., 10 min; FauI, 55° C., 10 min; BstYI, 60° C., 10 min; BsrI, 65° C., 10 min; EcoRI, 37° C., 10 min. 100 ng PCR products were used for each of reaction. The cleavage reactions were further analyzed by 1% agarose TAE gel.

FIG. 2A shows Schematic location of dZTP substituting region in PCR products. For DHFR and GFP, 441/480 bp and 671/720 bp of coding sequence were reprogramed by ZTGC.

FIG. 2B shows SDS-PAGE gel analysis of in vitro protein expression samples. 4-20% Tris-glycine gel with lane M, molecular weight marker, and lane 1,2,3,4 with no DNA, 250 ng plasmid, 250 ng DNA^{dATP} and 250 ng DNA^{dZTP}. DHFR, 18 kDa; GFP, 27 kDa. Reaction was carried out at 37° C. for 4 h. 5 μL sample were loaded for each.

FIG. 2C shows Bands intensity in (b) was analyzed by GelAnalyzer.

FIG. 2D shows Imaging fluorescence of GFP expression. Tube 1, 2, 3 and 4 with ddH₂O, no DNA, 250 ng DNA^{dATP} and 250 ng DNA^{dZTP}. The bottom is native in-gel analysis of GFP protein.

FIG. 2E shows Bands intensity in (d) was analyzed by GelAnalyzer.

FIG. 2F shows Schematic design of element used for EGFP expression in HEK293T cell. Element was amplified by PCR from pCMV-GFP plasmid.

FIG. 2G shows Flow cytometry analysis of HEK293 cell transfection. 50000 cells were seeded in each well of 24-well plate, about 30000 cells were input for detecting 48 h after 200 ng DNA transfection.

FIG. 3A shows a Schematic design of mRNA investigation.

FIG. 3B shows Gel analysis of Tailing products.

FIG. 3C shows Gel analysis of full-length transcripts.

FIG. 3D shows Flow cytometry analysis of HEK293 cell transfection. Cells were analyzed 48 h after transfection. 200 ng mRNA transfection. About 9000 cells were input for flow cytometry.

FIG. 3E shows Representative fluorescence images of cells.

FIG. 3F shows MFI analysis for (D) and (E).

FIGS. 4A-4D. Z base has the same fidelity to A base.

FIG. 4A. Workflow of preparing samples and NGS analysis. In the step 1, original DNA template was plasmid pMRNA-GFP. Taq polymerase was used to prepare PCR products. G111/G112 primers were used for PCR in step 1 and 2 reaction.

FIG. 4B. Coverage depth of each position.

FIG. 4C. Frequency of errors in reads.

FIG. 4D. Frequency of each type errors in (c).

FIG. 5A shows a Schematic of Cas9 sgRNA paired with target DNA. RNA is shown in thick, whereas DNA is in bold.

FIG. 5B shows Schematic showing the comparison of spacer position between DNA-dATP and DNA-dZTP substrate.

FIG. 5C shows Schematic of Cpf1 sgRNA paired with target DNA. RNA is shown in thick, whereas DNA is in bold.

FIG. 5D shows sgRNA yield comparison of IVT with ATP or ZTP.

FIG. 5E shows Gel analysis of Cas9 sgRNA.

FIG. 5F shows Cas9 can't use ZGTC sgRNA targeted DNA cleave.

FIG. 5G shows DNA amplicons containing dATP and dZTP-substitution were cleaved in vitro by programmed Cas9 along with sgRNA-ATP.

FIG. 5H shows Plasmid cleave assay of Cas9.

FIG. 5I and FIG. 5J shows Plasmid cleave assay of Cpf1.

FIG. 6 shows Table 2 pMRNA-GFP plasmid sequence.

FIGS. 7A and 7B show Gel and Spectra analysis of DNA-dATP and DNA-dZTP PCR products. PCR products were analyzed by TAE gel (FIG. 7A). lane 1, GFP-dATP, lane 2, GFP-dZTP, lane 3, DHFR-dATP, lane 4, GFP-dATP. The top was GFP amplified from pMRNA-GFP using primers G050/G051. The bottom was DHFR amplified from DHFR-His Control Plasmid using primers G052/G053.

FIG. 8 shows Schematic representation of GFP and DHFR. The top was GFP amplified from pMRNA-GFP using primers G050/G051. The bottom was DHFR amplified from DHFR-His Control Plasmid using primers G052/G053.

FIG. 9 shows Table 3 GFP plasmid for E. coli synthesized by IDT.

FIG. 10 shows Table 4 DNA used in cell free expression.

FIG. 11 shows In vitro expression of GFP using cell free system. eGFP-Human-dATP was amplified from pMRNA-GFP using primers G050/G051 with dATP. eGFP-Human-dZTP bottom was amplified from pMRNA-GFP using primers G050/G051 with dZTP. 250 ng DNA added into 25 μl reaction volume was used expression template.

FIG. 12 shows Alignment of two GFP coding sequence. Top is sequence optimized from E. coli codon, bottom is sequence optimized from human. eGFP-E. coli was amplified from Table 3 plasmid using primers G063/G051 with dATP or dZTP.

FIG. 13 shows Design of expression cassettes for investigation Z-substitution in whole length.

FIG. 14 shows Table 5 pCMV-GFP plasmid.

FIG. 15 shows Flow cytometry analysis of S. c cells transformed with eGFP expression cassettes DNA. Y-1, DNA-dATP; Y-2, DNA-dZTP; Y-3, negative control. BL1-H represents fluorescence intensity of GFP. About 500000 cells of each sample were input for analysis.

FIG. 16 shows MFI analysis for Z-substituted DNA expression in HEK293 cells.

FIG. 17 shows Representative fluorescence images of HEK293T cells with GFP DNA transfection in FIG. 2.

FIG. 18 shows MFI and transection efficiency analysis for mRNA with tailing Z in HEK293 cells. N=2, ***P=0.0003.

FIG. 19 shows Representative bright and fluorescence images of HEK293T cells with differing Tailing mRNA transfection.

FIG. 20 shows Table 6 PCR products for DNA cleavage using Cas9 in FIG. 5.

FIG. 21 shows In vitro cleavage of assay Cas9 using different sgRNA. PCR products were prepared by the same method with the No. 1 sequence in Table 4.

FIG. 22 shows Table 7 Primers used in this study.

FIG. 23 shows cleavage activity for each guide RNA with ATP or ZTP. The Y-axis is the percentage of cleavage, and the X-axis is the incubation time (min).

FIG. 24 is a schematic illustration of this research. Top left frame shows Z-U and G-C base pairs in RNA written by the ZUGC alphabet. Top right frame shows Z-T and G-C base pairs in DNA written by the ZTGC alphabet. LNP, lipid nanoparticle. ZTGC-DNA base pairs. Hydrogen bonds are marked by dotted lines. The additional amino group was highlighted in yellow.

FIGS. 25A-25E show comparison of A-DNA and Z-DNA properties. FIG. 25A shows the schematic strategy for NGS analysis of DNA amplicons. The artificial DNA1 sequence was used in this investigation. FIG. 25B shows the Depth of coverage and the percentage of correct reads at each base pair. FIG. 25C shows the frequency of errors-types in reads from FIG. 25B.

FIG. 25D shows the melting temperature analysis of A-DNA and Z-DNA. FIG. 25E shows the relative activity of different REs on A-DNA and Z-DNA. Relative cleavage activity in each group was normalized to that of the A-DNA (relative activity (%)=100). Data are presented as mean values for three replicates. The reaction was carried out according to the instructions of BsrFI, FauI, BstYI, BsrI, and EcoRI. 100 ng PCR products were used for each reaction. Top, content or amount of AT or ZT in sticky end of each cleave site. Bottom, recognition sequences were labeled in red. Cleavage sites are highlighted by red triangles. Error bars represent mean values±sd, n=3.

FIGS. 26A-26I show that ZTGC-DNA can be decoded in various life systems. FIG. 26A shows the schematic of Z-substituting region in PCR products used in FIG. 26B, FIG. 26C, FIG. 26D, FIG. 26E. Top, design of DNA construct; bottom, region written by ZTGC in Z-DNA. DHFR amplicons were amplified from a NEBExpress DHFR Control Plasmid template using G052/G053 primers. GFP amplicons were amplified from a pIDT-eGFP template using G063/G064 primers. FIG. 26B shows the analysis of in vitro protein expression samples of DHFR using different DNA templates on a gel. Reaction was carried out at 37° C. for 4 hr. 5 μl sample of each reaction was loaded for gel analysis. The graph represents 1 of 3 independent experiments. FIG. 26C shows the band intensities in FIG. 26B analyzed by GelAnalyzer. FIG. 26D: Top, imaging fluorescence of GFP expression by in vitro protein expression in a cell free system extracted from E. coli using different DNA templates. Bottom, in-gel fluorescence detection of eGFP protein. The graph represents 1 of 2 independent experiments. FIG. 26E shows the band intensities in FIG. 26D analyzed by GelAnalyzer. FIG. 26F shows the schematic construct design of DNA template used for eGFP expression in human cell. DNA was amplified by PCR from pCMV-GFP plasmid using G132/G133 primers. FIG. 26G shows the representative flow cytometry plots of HEK293T cells 48 hr post-transfection. About 30,000 cells were used as input for flow cytometry. Top left, negative control; bottom left, transfection with pCMV-GFP plasmid; top right, transfection with A-DNA; bottom right, transfection with Z-DNA. 200 ng DNA was used for each transfection. FIG. 26H shows hela cells 48 hr post-transfection analyzed by flow cytometry and gated on GFP+transfected cells. About 30,000 cells were used as input for flow cytometry. Left, negative control; center, transfection with A-DNA; right, transfection with Z-DNA. 200 ng DNA was used for each transfection. The graph represents 1 of 3 independent experiments. FIG. 26I summarizes the MFI and percent of GFP+ cells. Error bars represent mean values±sd, n=2 or 3.

FIGS. 27A-27I show that ZUGC-mRNA enables specific protein expression in human cells. FIG. 27A shows the schematic strategy of mRNA investigation. Top, DNA template amplified from a pMRNA-eGFP plasmid using G126/G127 paired primers. RNA transcripts were produced by in vitro transcription (IVT) with standard nucleotides. 5 μg RNA substrate was added into tailing reactions with either ATP or ZTP to synthesize mRNA^{poly(A)} and mRNA^{poly(Z)}, respectively. Bottom, DNA template amplified from a pMRNA-GFP plasmid using a tail primer mix. This tailing DNA template was added into in vitro transcription reactions with ZTP or ATP to produce full length RNA transcripts. mRNA products were reverse transcribed into cDNA, which were used as templates for PCR. PCR amplicons were sequenced by an illumina NGS platform. FIG. 27B shows the denaturing gel analysis of mRNA with no tail, a poly(A) tail, or a poly(Z) tail. 360 ng of each mRNA was loaded for gel detection. ssRNA ladder was used. FIG. 27C shows the denaturing gel analysis of full-length transcript mRNA quality. IVT was performed with either ATP or ZTP. ssRNA ladder was used. 200 ng of each mRNA was loaded. FIG. 27D shows the frequency of errors in reads from mRNA templates. FIG. 27E shows HEK293T cells 24 hr post-transfection analyzed by flow cytometry and gated on GFP+transfected cells. Cells were transfected with 200 ng of AUGC-mRNA or ZUGC-mRNA. About 9,000 cells were used as input for flow cytometry. FIG. 27F shows the representative fluorescence images of HEK293T cells 24 hr post-transfection with 200 ng A-mRNA or Z-mRNA. FIG. 27G summarizes the MFI and percent of GFP+ cells. FIG. 27H is the schematic figure of mRNAs carrying different stop codons. FIG. 27I are representative flow cytometry plots of HEK293T cells 24 hr post-transfection with 58_UGG_mRNA, 58_UAG_mRNA, and 58_UZG_mRNA, respectively. Error bars represent mean values±sd, n=3.

FIGS. 28A-28J show that ZUGC-crRNA can guide Cas12a to cleave plasmids and PCR products. FIG. 28A shows the schematic map of plasmid used in FIG. 28B, FIG. 28C, and FIG. 28D. The frame representation of the Z-crRNA-DNA-targeting complex. Blue, targeted region and crRNA sequence; purple, PAM motif (5′-TTTA); red bonds, non-Watson-Crick base pairs in the pseudoknot structure of Cas12a Z-crRNA; italicized and underlined, seed region; grey bonds, Watson-Crick base pairs. FIG. 28B shows the cleavage assay of plasmid DNA mediated by A-crRNA and Z-crRNA. Reactions incubated at 37° C. for 30 min. 200 ng plasmid was used for each reaction. FIG. 28C shows the comparison of cleavage assay of supercoiled DNA. Reaction was incubated at 37° C. for 30 min. FIG. 28D shows the sanger sequencing of cleavage products from FIG. 28C. The non-templated addition of an additional adenine, denoted as N, is an artifact of the polymerase used in sequencing. FIG. 28E shows the schematic locations of guide sequences used in FIG. 28F and FIG. 28G for this assay. DNA substrates were amplified from a pIDT-DNA4 plasmid using G030/G031 primers. FIG. 28F shows the characteristics of each guide sequence used for FIG. 28G. Seed regions are in blue. FIG. 28G shows both Z-crRNA and A-crRNA guide Cas12a to cleave Z-DNA and A-DNA PCR products. Graph shows cleavage % of reaction with each crRNA. Bottom, % A/Z and % AT/ZT in guide sequence. 300 ng DNA was used for each reaction incubated for 30 min at 37° C. FIG. 28H shows the characteristics of each guide sequence used for FIG. 28I. Seed regions are in blue. FIG. 28I shows the quantified time-course data of cleavage by Cas12a loaded with A-crRNA or Z-crRNA. SspI linearized pIDT-DNA4 plasmid was used as DNA substrates. FIG. 28J shows the sequence logo for the top eight target sites in FIG. 28H and FIG. 28I. Colored nucleotides match the most common nucleotide at that position. Analysis was done with WebLogo3.7.12. Error bars represent mean values±sd, n=3.

FIGS. 29A-29I shows that SpCas9 is guided by Z-crRNA to cleave A-DNA and Z-DNA. FIG. 29A shows the schematic of standard crRNA-tracrRNA of SpCas9 paired with target standard DNA. DNA and RNA nucleotides are shown in bold and light, respectively. Red bonds, non-Watson-Crick base pairs; grey bonds, Watson-Crick base pairs. FIG. 29B shows the schematic structure of A-sgRNA for SpCas9. FIG. 29C shows the cleavage assay of plasmid with using different crRNA and tracrRNA. 170 ng plasmid pIDT-EMX1 was used for each reaction. Reactions were incubated at 37° C. for 1 hr. FIG. 29D shows the schematic locations of guide sequence used in (FIG. 29E) for this assay. DNA substrates were amplified from a pIDT-EMX1 plasmid using G030/G031 primers. The frame schema shows the comparison between protospacer positions in A-DNA and Z-DNA substrates. Blue, non-target strand in A-DNA; light blue, non-target strand in Z-DNA; green, target strand in A-DNA; light green, target strand in Z-DNA; purple, crRNA for Cas9; orange, tracrRNA; red, PAM motif (GGG). FIG. 29E shows the PCR DNA cleavage assay of Cas9 with either A-crRNA or Z-crRNA. 300 ng DNA substrate per reaction was used. Reactions were incubated at 37° C. for 30 min. FIG. 29F shows the characteristics of each guide sequence used for FIG. 29G. Seed regions are in blue. FIG. 29G shows cleavage % of each crRNA. Error bars represent mean values±sd, n=3. FIG. 29H shows the relative activity of Z-crRNA to A-crRNA for Cas12a and Cas9. FIG. 29I shows the relative activity on ZTGC-DNA to ATGC-DNA. Values for EcoR1, Bsrl, FauI, and BstY1 were summarized from FIG. 25E. Values for LbCas12a were summarized from FIG. 28G. Values for SpCas9 were summarized from FIGS. 29E and 29G. Values for LbCas12a and SpCas9 only refer to its cleavage ability with A-crRNA or A-sgRNA.

FIGS. 30A-30D show gene editing with ZUGC-crRNA in human cells. FIG. 30A shows the EMX1 gene editing efficiency withCas9. Cells were transfected with a SpCas9 mRNA and its corresponding Z-crRNA:A-tracrRNA and A-crRNA:A-tracrRNA. FIG. 30B shows the indel-pattern % in total indel reads. FIG. 30C shows the A-to-G base editing efficiency of ABE8e with A-crRNA or Z-crRNA. All values are presented as mean±sd, n=2 or 3. FIG. 30D shows the Cas9-dependent DNA off-target analysis of the indicated sites (EMX1 site 1, ABE site 6). HeLa cells were used for this test. Error bars represent mean values±sd, n=2 or 3.

FIG. 31 shows the artificial DNA sequence (DNA1) used in FIG. 25.

FIGS. 32A-32C show the PCR yield analysis. FIG. 32A shows the relative PCR yield with or without dZTP. FIG. 32B shows the length and GC % of PCR amplicons. FIG. 32C shows the correlation between relative PCR yield and GC %. For each PCR test, the yield of PCR amplicons with dATP was set as 100%. Template sequences and primers used in this figure were shown in Table 9.

FIG. 33 shows the summarized statistical-analysis of errors-frequency in next generation sequencing data from A-DNA or Z-DNA templates for each kind of nucleotide.

FIG. 34 shows the sequence of DNA2. ZTGC-DNA2, AT pairs in 676 bp bases highlighted were flanked by primers G063/G064.

FIGS. 35A-35F show the DNA sequences. FIG. 35A shows the ZTGC-DNA3. AT pairs in 441 bp bases highlighted were flanked by primers G052/G053. FIG. 35B shows the 16 bp DNA. AT pairs in red were replaced with ZT. FIG. 35C shows the18 bp DNA. AT pairs in red were replaced with ZT. FIG. 35D shows the 193 bp DNA. AT pairs in the region highlighted were flanked by primers G083/G084. FIG. 35E shows the 513 bp DNA. AT pairs in the region highlighted were flanked by primers G097/G098. FIG. 35F shows the 702 bp DNA. AT pairs in the region highlighted were flanked by primers G097/G098.

FIGS. 36A-36B show the melting curves of ZTGC-DNA and ATGC-DNA. FIG. 36A shows that each value pot represents the mean value of 3 independent replicates. A total of 442 mean value pots were used for each sample. FIG. 36B shows the representative melting curves of FIG. 25D. Each value pot represents the mean value of 5 independent replicates. The graph represents 1 of 3 replicates.

FIGS. 37A-37B show the in vitro DNA cleavage assay with restriction endonuclease. FIG. 37A shows the locations of recognition sites of restriction endonucleases in DNA2 and DNA3. FIG. 37B shows the gel analysis of cleavage assay. The graph represents 1 of 3 replicates.

FIG. 38 shows the natural codon usage for E. coli and H. sapiens. An adaptation from Snapgene.

FIG. 39 shows the alignment of two eGFP coding sequences. Top, coding sequence optimized from E. coli codon usage. Bottom, coding sequence optimized from H. sapiens codon usage.

FIG. 40 shows the in vitro expression of eGFP using cell free system. ATGC-DNA was amplified from pMRNA-eGFP plasmid using primers G050/G051 with standard nucleotides. ZTGC-DNA was amplified from pMRNA-eGFP plasmid using primers G050/G051 with dZTP. 250 ng DNA was added into each 25 μL reaction volume.

FIGS. 41A-41C show the compatible assay of ZTGC-DNA in Saccharomyces cerevisiae. FIG. 41A shows the design of construct for S. cerevisiae. FIG. 41B shows the purified PCR products of eGFP expression cassettes for S. cerevisiae. 120 ng DNA loaded in gel stained by SYBR safe. FIG. 41C shows the flow cytometry analysis of S. cerevisiae cells transformed with cassettes DNA in FIG. 41A. About 500,000 cells for each sample were used as input for analysis.

FIGS. 42A-42C show the investigation of compatibility of HEK293T cells with Z-DNA. FIG. 42A shows the architecture of DNA construct used for HEK293T cells transfection. Primers located outside the cassettes were used to amplify the normal or Z-substituted DNA strands. FIG. 42B shows the mean fluorescence intensity (MFI) and GFP+% analysis for Z-substituted DNA expression in HEK293T cells. All values are presented as mean±sd., n=3.

FIG. 42C shows the representative fluorescent images of HEK293T cells transfected with eGFP DNA in FIG. 25.

FIG. 43 shows the compatible assay of ZTGC-DNA for Fulc in HEK293T cells. Luciferase activity assay, all values are presented as mean±sd, n=3.

FIGS. 44A-44B show the investigation of mRNA with poly(Z) tail. FIG. 44A shows the mean fluorescence intensity (MFI) and GFP+efficiency analysis for GFP mRNA with no tail, a poly(A) tail, or a poly(Z) tail in HEK293T cells at 24 h post transfection. All values are presented as mean±sd, n=3. FIG. 44B shows the representative brightfield and fluorescence images of HEK293T cells after mRNA transfection. Top, no mRNA; middle, GFP mRNA without any tail; bottom, GFP mRNA with poly(Z) tail.

FIG. 45 shows the schematic of T>A substitution errors generated from in vitro transcription.

FIG. 46 shows the sequence of DNA4. The total sequence consists of 71.3% AT base pairs.

FIG. 47 shows the cleavage assay of PCR products with LbCas12a. DNA substrates were amplified from pIDT-DNA4 plasmid using G030/G031 primers. The graph represents 1 of 3 replicates.

FIG. 48 shows the cleavage assay of DNA4 plasmid with LbCas12a.

FIG. 49 shows the serum stability assay of crRNAs. 480 ng RNA was used for each reaction. Then the reaction was denatured and loaded in gel for electrophoresis. FBS, fetal bovine serum.

FIG. 50 shows the cleavage assay of pIDT-EMX1 plasmid with LbCas12a. 300 ng plasmid was used in each reaction.

FIGS. 51A-51B show the cleavage assay of pIDT-EMX1 plasmid with SpCas9. FIG. 51A shows the gel analysis of cleavage assay with Cas9 loaded by different guide RNAs. FIG. 51B shows the gel analysis of Cas9 sgRNA. Lane 1, low range ssRNA ladder. 160 ng sgRNA was loaded onto a 2% agarose gel.

FIGS. 52A-52E show that SpCas9 is guided by normal sgRNA to cleave A-DNA and Z-DNA. FIG. 52A shows the schematic of Cas9 sgRNA paired with target DNA. Plasmid pMRNA-eGFP was used in FIG. 52B. DNA and RNA nucleotides are shown in bold and light, respectively. FIG. 52B. Plasmid cleavage assay with Cas9 and either A-sgRNA or Z-sgRNA. 170 ng plasmid pMRNA-GFP was used for each reaction. Reactions were incubated at 37° C. for 1 hr. FIG. 52C show the schematic showing the comparison between protospacer positions in A-DNA and Z-DNA substrates. Blue, non-target strand in A-DNA; light blue, non-target strand in Z-DNA; green, target strand in A-DNA; light green, target strand in Z-DNA; purple, A-sgRNA for Cas9; red, PAM motif (AGG). FIG. 52D shows the PCR DNA cleavage assay with Cas9 and either A-sgRNA or Z-sgRNA. 300 ng A-DNA substrate per reaction was used. Reactions were incubated at 37° C. for 30 min. FIG. 52E shows the DNA amplicons containing dATP or dZTP-substitutions were cleaved in vitro by Cas9 with either A-sgRNA or Z-sgRNA. *, 30 ng sgRNA per reaction; **, 60 ng sgRNA per reaction. Reactions were incubated at 37° C. for 1 hr. PCR DNA used in FIG. 52D and FIG. 52E were amplified from pMRNA-eGFP plasmid using G001/G002 primers.

FIG. 53 shows the sequence of DNA5 template.

FIGS. 54A-54C show the cleavage assay of PCR DNA. FIG. 54A shows the schematic locations of guide sequences used in FIG. 54B and FIG. 29F and FIG. 29G for this assay. DNA substrates were amplified from a pIDT-DNA4 plasmid using G030/G031. FIG. 54C shows DNA substrates amplified from a pIDT-DNA6 plasmid using G097/G098 primers.

FIG. 55 shows the cleavage assay of linear plasmid DNA substrates. pIDT-DNA4 plasmid was linearized by SspI.

FIG. 56 shows the distribution of A-to-G frequencies (>1%) in the protospacer from tested 6 sites with ABE8e using A-crRNA and Z-crRNA.

### DETAILED DESCRIPTION OF THE INVENTION

In some aspects, the present disclosure provides compositions (e.g., nucleic acids) that comprise one or more Z-bases and their uses in protein expression and/or gene editing. It was demonstrated herein that both ZTGC-DNA (Z-DNA) and ZUGC-RNA (Z-RNA) produced in vitro show detectable compatibility and can be decoded in mammalian cells, including H. sapiens cells. The present disclosure also shows that Z-crRNA can guide clustered regularly interspaced short palindromic repeat (CRISPR)-effectors SpCas9 and LbCas12a to cleave specific DNA through non-Watson-Crick base pairing and boost cleavage activities compared to A-crRNA. Z-crRNA can also allow for efficient gene and base editing in human cells. Together, the present disclosure paves the way for new strategies for optimizing DNA or RNA payloads for gene editing therapeutics and guides the rational design of improved nucleic acid-based therapies such as CRISPR genome editing by expanding the possible types of nucleotide modifications.

### Definitions

For convenience, certain terms employed in the specification, examples, and appended claims are collected here.

The articles “a” and “an” are used herein to refer to one or to more than one (e.g., to at least one) of the grammatical object of the article. By way of example, “an element” means one element or more than one element.

As used herein, two nucleic acid sequences “complement” one another or are “complementary” to one another if they base pair one another at each position.

The term “polynucleotide” and “nucleic acid” refer to a polymeric form of nucleotides of any length, either deoxyribonucleotides or ribonucleotides, or analogs thereof. It may be composed of four standard nucleotides, each with a different nucleobase: adenine (A), thymine (T)/Uridine (U), guanine (G), and cytosine (C). It may contain non-conventional nucleobases, such as the Z-base. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

“Cas12a” a subtype of Cas12 proteins and an RNA-guided endonuclease that forms part of the CRISPR system in some bacteria and archaea. Cas12a is formerly known as Cpf1, and the terms “Cas12a” and “Cpf1” are used interchangeably herein. In some embodiments, Cas12a is a LbCas12a from Lachnospiraceae bacterium, which is a Type V CRISPR associated protein (Cas) effector that prefers a T-rich 5′-TTTN Protospacer Adjacent Motif (PAM). The single guide RNA (gRNA) used for LbCas12a consists only of a 39-nt CRISPR RNA (crRNA). In this case, the terms “Cas12a crRNA”, “Cpf1 crRNA”, “Cas12a gRNA”, and “Cfp1 gRNA” are used interchangeably herein. The “seed region” of Cas 12a refers to the first 5 nucleotides at the 5′-end of the guide sequence which are complimentary to the target DNA.

The term “modifying” a target DNA refers to changing the sequence of the target DNA, for example, by introducing a deletion, an insertion, and/or a substitution of the target DNA sequence. In some embodiments, “modifying” a target DNA refers to inducing an indel in the target DNA. In some embodiments, “modifying” a target DNA refers to inducing a base change, e.g., a A-to-G base change in the target DNA.

### Nucleic Acids

In some aspects, provided herein is a nucleic acid (e.g., DNA or RNA) comprising at least one 2-aminoadenine (Z) base. The nucleic acid can be either single-stranded or double-stranded. The nucleic acid can be either circular or linear.

In some embodiments, the nucleic acid comprising at least one Z base is double-stranded and is at least 2.5 kilobase (kb) in length, for example, at least 2.4 kb, at least 2.3 kb, at least 2.2 kb, at least 2.1 kb, at least 2 kb, at least 1.9 kb, at least 1.8 kb, at least 1.7 kb, at least 1.6 kb, at least 1.5 kb, at least 1.4 kb, at least 1.3 kb, at least 1.2 kb, at least 1.1 kb, at least 1 kb, at least 0.9 kb, at least 0.8 kb, at least 0.7 kb, at least 0.6 kb, at least 500 bp, at least 450 bp, at least 400 bp, at least 350 bp, at least 300 bp, at least 250 bp, at least 200 bp, at least 150 bp, at least 100 bp, at least 50 bp, at least 20 bp, or at least 10 bp in length.

In some embodiments, the nucleic acid comprising at least one Z base is double-stranded and is no more than 10 kb, no more than 9 kb, no more than 8 kb, no more than 7 kb, no more than 6 kb, no more than 5 kb, no more than 4.5 kb, no more than 4 kb, no more than 3.5 kb, no more than 3 kb, no more than 2 kb, no more than 1.9 kb, no more than 1.8 kb, no more than 1.7 kb, no more than 1.6 kb, no more than 1.5 kb, no more than 1.4 kb, no more than 1.3 kb, no more than 1.2 kb, no more than 1.1 kb, no more than 1 kb, no more than 0.9 kb, no more than 0.8 kb, no more than 0.7 kb, no more than 0.6 kb, or no more than 500 bp in length.

In some embodiments, the nucleic acid comprising at least one Z base is double-stranded and is from 50 bp to 5 kb in length, for example, from 100 bp to 2.5 kb, from 100 bp to 2.0 kb, from 100 bp to 1.8 kb, from 100 bp to 1.6 kb, from 100 bp to 1.5 kb, from 100 bp to 1.2 kb, from 100 bp to 1.0 kb, from 100 bp to 900 bp, from 100 bp to 800 bp, from 100 bp to 700 bp, from 100 bp to 600 bp, from 100 bp to 500 bp, from 100 bp to 250 bp, from 200 bp to 2.5 kb, from 200 bp to 2.0 kb, from 200 bp to 1.5 kb, from 200 bp to 1.0 kb, from 200 bp to 500 bp, from 500 bp to 2.5 kb, from 500 bp to 2.0 kb, from 500 bp to 1.5 kb, from 500 bp to 1.0 kb, from 1 kb to 2.5 kb, from 1 kb to 2.0 kb, from 1 kb to 1.5 kb in length.

In some embodiments, the nucleic acid comprising at least one Z base is double-stranded and is about 10 kb in length, for example, about 9 kb, about 8 kb, about 7 kb, about 6 kb, about 5 kb, about 4.5 kb, about 4 kb, about 3.5 kb, about 3 kb, about 2 kb, about 1.9 kb, about 1.8 kb, about 1.7 kb, about 1.6 kb, about 1.5 kb, about 1.4 kb, about 1.3 kb, about 1.2 kb, about 1.1 kb, about 1 kb, about 0.9 kb, about 0.8 kb, about 0.7 kb, about 0.6 kb, about 500 bp, about 450 bp, about 400 bp, about 350 bp, about 300 bp, about 250 bp, about 200 bp, about 150 bp, about 100 bp, about 50 bp, or about 20 bp in length.

In some embodiments, the nucleic acid comprising at least one Z base is single-stranded and comprises at least 2500 nucleotides (nt), for example, at least 2400 nt, at least 2300 nt, at least 2200 nt, at least 2100 nt, at least 2000 nt, at least 1900 nt, at least 1800 nt, at least 1700 nt, at least 1600 nt, at least 1500 nt, at least 1400 nt, at least 1300 nt, at least 1200 nt, at least 1100 nt, at least 1000 nt, at least 900 nt, at least 800 nt, at least 700 nt, at least 600 nt, at least 500 nt, at least 450 nt, at least 400 nt, at least 350 nt, at least 300 nt, at least 250 nt, at least 200 nt, at least 150 nt, at least 100 nt, at least 50 nt, at least 20 nt, or at least 10 nt.

In some embodiments, the nucleic acid comprising at least one Z base is single-stranded and comprises no more than 10000 nucleotides (nt), no more than 9000 nt, no more than 8000 nt, no more than 7000 nt, no more than 6000 nt, no more than 5000 nt, no more than 4500 nt, no more than 4000 nt, no more than 3500 nt, no more than 3000 nt, no more than 2000 nt, no more than 1900 nt, no more than 1800 nt, no more than 1700 nt, no more than 1600 nt, no more than 1500 nt, no more than 1400 nt, no more than 1300 nt, no more than 1200 nt, no more than 1100 nt, no more than 1000 nt, no more than 900 nt, no more than 800 nt, no more than 700 nt, no more than 600 nt, or no more than 500 nt.

In some embodiments, the nucleic acid comprising at least one Z base is single-stranded and comprises from 50 to 5000 nt, for example, from 100 to 2500 nt, from 100 to 2000 nt, from 100 to 1800 nt, from 100 to 1600 nt, from 100 to 1500 nt, from 100 to 1200 nt, from 100 to 1000 nt, from 100 to 900 nt, from 100 to 800 nt, from 100 to 700 nt, from 100 to 600 nt, from 100 to 500 nt, from 100 to 250 nt, from 200 to 2500 nt, from 200 to 2000 nt, from 200 to 1500 nt, from 200 to 1000 nt, from 200 to 500 nt, from 500 to 2500 nt, from 500 to 2000 nt, from 500 to 1500 nt, from 500 to 1000 nt, from 1000 to 2500 nt, from 1000 to 2000 nt, from 1000 to 1500 nt in length.

In some embodiments, the nucleic acid comprising at least one Z base is single-stranded and comprises about 10000 nucleotides (nt), about 9000 nt, about 8000 nt, about 7000 nt, about 6000 nt, about 5000 nt, about 4500 nt, about 4000 nt, about 3500 nt, about 3000 nt, about 2000 nt, about 1900 nt, about 1800 nt, about 1700 nt, about 1600 nt, about 1500 nt, about 1400 nt, about 1300 nt, about 1200 nt, about 1100 nt, about 1000 nt, about 900 nt, about 800 nt, about 700 nt, about 600 nt, about 500 nt, about 450 nt, about 400 nt, about 350 nt, about 300 nt, about 250 nt, about 200 nt, about 150 nt, about 100 nt, about 50 nt, or about 20 nt.

In some embodiments, at least 1% nucleotides of the nucleic acid provided herein comprise a 2-aminoadenine (Z) base. For example, at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 82%, 85%, 88%, 90%, 95%, 98%, or 99% nucleotides of the nucleic acid provided herein comprise a 2-aminoadenine (Z) base.

In some embodiments, the nucleic acid provided herein is double-stranded and comprises at least 1% ZT content, i.e., at least 1% Z and T bases. For example, at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 82%, 85%, 88%, 90%, 95%, 98%, or 99% ZT content.

In some aspects, provided herein is a nucleic acid comprising less than or equal to 2500 nucleotides (e.g., less than or equal to 2400 nt, 2300 nt, 2200 nt, 2100 nt, 2,000 nt, 1900 nt, 1800 nt, 1700 nt, 1600 nt, 1500 nt, 1400 nt, 1300 nt, 1200 nt, 1100 nt, 1000 nt, 900 nt, 800 nt, 700 nt, 600 nt, 500 nt, 400 nt, 300 nt, or 200 nt), wherein at least 15% (e.g., at least 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, or 77%) of said nucleotides comprise a 2-aminoadenine (Z) base.

In some aspects, provided herein is a nucleic acid that is less than or equal to 2500 bp in length (e.g., less than or equal to 2400 bp, 2300 bp, 2200 bp, 2100 bp, 2,000 bp, 1900 bp, 1800 bp, 1700 bp, 1600 bp, 1500 bp, 1400 bp, 1300 bp, 1200 bp, 1100 bp, 1000 bp, 900 bp, 800 bp, 700 bp, 600 bp, 500 bp, 400 bp, 300 bp, or 200 bp in length) and comprises at least 15% (e.g., e.g., at least 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, or 77%) of ZT content.

In some embodiments, the nucleic acid comprises one or more adenine (A) bases. In some embodiments, the nucleic acid does not comprise any adenine (A) base. In some embodiments, the nucleic acid comprises Z, A, U, G, C bases. In some embodiments, the nucleic acid comprises Z, A, T, G, C bases. In some embodiments, the nucleic acid comprises Z, U, G, C bases. In some embodiments, the nucleic acid comprises Z, T, G, C bases.

In some embodiments, the nucleic acid is a DNA, e.g., a genomic DNA, a synthetic DNA, a plasmid, a DNA comprising at least one intron, or a cDNA. In some embodiments, the nucleic acid is a RNA, e.g., a mRNA, a crRNA, a tracrRNA, or a guide RNA (gRNA). In some embodiments, the nucleic acid is an mRNA comprising a poly(Z) tail.

In some embodiments, the nucleic acid provided herein further comprises at least one chemical modification.

In some embodiments, the nucleic acid comprises a terminal modification. In some embodiments, the nucleic acid is chemically modified with poly-ethylene glycol (PEG) (e.g., attached to the 5′ end of the nucleic acid). In some embodiments, the nucleic acid comprises a 5′ end cap (e.g., an inverted thymidine, biotin, albumin, chitin, chitosan, cellulose, terminal amine, alkyne, azide, thiol, maleimide, NHS). In certain embodiments, the nucleic acid comprises a 3′ end cap (e.g., an inverted thymidine, biotin, albumin, chitin, chitosan, cellulose, terminal amine, alkyne, azide, thiol, maleimide, NHS).

In some embodiments, the nucleic acid provided herein comprises one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50) modified sugars. In some embodiments, the nucleic acid provided herein comprises one or more 2′ sugar substitutions (e.g., a 2′-fluoro, a 2′-amino, or a 2′-O-methyl substitution). In certain embodiments, the nucleic acid provided herein comprises one or more locked nucleic acid (LNA), unlocked nucleic acid (UNA), peptide nucleic acid (PNA), and/or 2′deozy-2′fluoro-D-arabinonucleic acid (2′-F ANA) sugars in their backbone.

In certain embodiments, the nucleic acid comprises one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, 35, 36, 37, 38, 39, 40, 41, 42, 43, 44, 45, 46, 47, 48, 49, 50) methylphosphonate internucleotide bonds and/or phosphorothioate (PS) internucleotide bonds.

In some aspects, provided herein is a vector comprising a nucleic acid described herein. In some embodiments, the vector is an expression vector. In some embodiments, the vector is a plasmid. In some embodiments, the vector is a viral vector.

The nucleic acids and/or vectors described herein can be used for methods for expressing or producing protein, or methods for modifying a target DNA described below.

### Methods for Protein Expression

In some aspects, provided herein is a method of expressing or producing a protein, comprising contacting a cell with a nucleic acid comprising a nucleotide sequence encoding the protein, wherein the nucleic acid comprises at least one Z base.

In some embodiments, the nucleic acid is a DNA, e.g., a genomic DNA, a cDNA, or a plasmid DNA. The DNA can be a circular DNA or a linear DNA, and can be single-stranded or double stranded DNA. In some embodiments, the nucleic acid is a mRNA. In some embodiments, the nucleic acid is a vector, e.g., an expression vector (e.g., a plasmid or a viral vector).

In some embodiments, the cell is a prokaryotic cell (e.g., a E. Coli cell) or a eukaryotic cell (e.g., a 293T cell or Hela cell).

### Compositions and Methods for Genome Editing

### CRISPR-Cas12a/Cpf1 Systems

In some aspects, provided herein is a composition comprising: (a) a Cas12a RNA-guided endonuclease or a nucleic acid encoding the Cas12a RNA-guided endonuclease; and (b) a Cas12a crRNA comprising at least one Z base.

In some aspects, provided herein is a kit comprising: (a) a Cas12a RNA-guided endonuclease or a nucleic acid encoding the Cas12a RNA-guided endonuclease; and (b) a Cas12a crRNA comprising at least one Z base.

In some aspects, provided herein is a method of cleaving or modifying a target DNA, comprising: contacting the target DNA or a cell comprising the target DNA with: (a) a Cas12a RNA-guided endonuclease, or a nucleic acid encoding the Cas12a RNA-guided endonuclease; and (b) a Cas12a crRNA comprising at least one Z base, wherein the Cas12a RNA-guided endonuclease and the Cas12a crRNA form a complex that cleaves or modifies the target DNA. In some embodiments, the target DNA is a plasmid DNA.

In some embodiments, the Cas12a crRNA comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 Z bases. In some embodiments, the Cas12a crRNA comprises 1, 2, 3, 4, 5, 6, 7, 8, 9, 10, 11, 12, 13, 14, 15, 16, 17, 18, 19, 20, 21, 22, 23, 24, or 25 Z bases. In some embodiments, the Cas12a crRNA does not comprise an A base.

In some embodiments, the Cas12a RNA-guided endonuclease is LbCas12a.

In some embodiments, the Cas12a crRNA comprising at least one Z base induces higher cleavage of the target DNA compared to the corresponding Cas12a crRNA where the at least one Z base is substituted with A base.

In some embodiments, the Cas12a crRNA comprising at least one Z base induces at least 1.1-fold (for example, at least 1.2-fold, at least 1.4-fold, at least 1.6-fold, at least 1.8-fold, at least 2.0-fold, at least 2.2-fold, at least 2.4-fold, at least 2.6-fold, at least 3.0-fold, at least 3.5-fold, at least 4-fold, at least 4.5-fold, at least 5-fold, at least 5.5-fold, at least 6-fold, at least 7-fold, at least 8-fold, at least 9-fold, or at least 10-fold) cleavage of the target DNA compared to the corresponding Cas12a crRNA where the at least one Z base is substituted with A base.

In some embodiments, the Cas12a crRNA comprising at least one Z base induces about 1.1-fold, about 1.2-fold, about 1.4-fold, about 1.6-fold, about 1.8-fold, about 2.0-fold, about 2.2-fold, about 2.4-fold, about 2.6-fold, about 3.0-fold, about 3.3-fold, about 3.5-fold, about 4-fold, about 4.5-fold, about 5-fold, about 5.5-fold, about 6-fold, about 7-fold, about 8-fold, about 9-fold, or about 10-fold) cleavage of the target DNA compared to the corresponding Cas12a crRNA where the at least one Z base is substituted with A base.

In some embodiments, the Cas12a crRNA comprises at least one (e.g., 1, 2, 3, 4, or 5) Z base in the seed region.

In some embodiments, the Cas12a crRNA comprises at least one (e.g., 1, 2, 3, 4, 5, 6, 7, 8, 9, 10, 11, 12, 13, 14, 15, 16, 17, 18, 19, or 20) Z base in the 20-nt guide sequence.

In some embodiments, the Cas12a crRNA comprises a 20-nt guide sequence complimentary to a target DNA site with an A or Z content from 5% to 95%, between 5% to 60%, between 5% to 50%, between 10% to 90%, between 10% to 40%, between 15% to 75%, between 15% to 35%, between 20% to 70%, between 20% to 30%, between 25% to 65%, between 35% to 60%, or between 40% to 55%.

In some embodiments, the Cas12a crRNA comprises a 20-nt guide sequence complimentary to a target DNA site with an AT or ZT content between 35% to 50%, between 35% to 55%, between 35% to 60%, between 35% to 65%, between 35% to 70%, between 35% to 75%, between 35% to 85%, or between 35% to 90%, between 35% to 95%, between 45% to 50%, between 45% to 55%, between 45% to 60%, between 45% to 65%, between 45% to 70%, between 45% to 75%, between 45% to 85%, or between 45% to 90%, or between 45% to 95%.

In some embodiments, the target DNA comprises a 5′-TTTA, 5′-TTTC, or 5′-TTTG PAM motif. In some embodiments, the target DNA comprises a T-base at position 5′-PAM-6, 8, 10, 18, 19-3′.

In some embodiments, the target DNA comprises at least one Z base. In some embodiments, the target DNA comprises at least one Z base at the region that is complimentary to the 20-nt guide sequence of the Cas12a crRNA. In some embodiments, the target DNA comprises at least one (e.g., 1, 2, 3, 4, 5, 6, 7, 8, 9, 10, 11, 12, 13, 14, 15, 16, 17, 18, 19, or 20) Z base at the region that is complimentary to the 20-nt guide sequence of the Cas12a crRNA.

In some embodiments, the target DNA is the genomic DNA. In some embodiments, the target DNA is a plasmid DNA.

In some embodiments, the cell comprising the target DNA is a mammalian cell.

In some aspects, provided herein is a method of improving cleavage activity or editing efficiency of a complex comprising a Cas12a RNA-guided endonuclease and a Cas12a crRNA, comprising substituting at least one (e.g., 1, 2, 3, 4, 5, 6, 7, 8, 9, 10, 11, 12, 13, 14, 15, 16, 17, 18, 19, 20, 21, 22, 23, 24, or 25) A base of the Cas12a crRNA with a Z base. In some embodiments, the method comprises substituting all A bases of the Cas12a crRNA with Z bases.

In some embodiments, the method comprises substituting at least one A base of the DNA substrate with a Z base. In some embodiments, the method comprises substituting all A bases of the DNA substrate with Z bases.

In some embodiments, the method comprises substituting at least one (e.g., 1, 2, 3, 4, 5, 6, 7, 8, 9, 10, 11, 12, 13, 14, 15, 16, 17, 18, 19, or 20) A base of the region that is complimentary to 20-nt guide sequence of the Cas12a crRNA of the DNA substrate with a Z base. In some embodiments, the method comprises substituting all A bases of the region that is complimentary to 20-nt guide sequence of the Cas12a crRNA of the DNA substrate with Z bases.

In some aspects, provided herein is a method of cleaving or modifying a target DNA comprising at least one Z base, comprising: contacting the target DNA or a cell comprising the target DNA with: (a) a Cas12a RNA-guided endonuclease or a nucleic acid encoding the Cas12a RNA-guided endonuclease; and (b) a Cas12a crRNA or a nucleic acid encoding a Cas12a crRNA, wherein the Cas12a RNA-guided endonuclease and the Cas12a crRNA form a complex that cleaves or modifies the target DNA comprising at least one Z base.

In some embodiments, the target DNA is the genomic DNA. In some embodiments, the target DNA is a plasmid DNA. In some embodiments, the target DNA comprises at least one Z base at the region that is complimentary to the 20-nt guide sequence of the Cas12a crRNA. In some embodiments, the target DNA comprises at least one (e.g., 1, 2, 3, 4, 5, 6, 7, 8, 9, 10, 11, 12, 13, 14, 15, 16, 17, 18, 19, or 20) Z base at the region that is complimentary to the 20-nt guide sequence of the Cas12a crRNA.

In some embodiments, the target DNA comprises a Z content between 5% to 95%, between 5% to 60%, between 5% to 50%, between 10% to 90%, between 10% to 40%, between 15% to 75%, between 15% to 35%, between 20% to 70%, between 20% to 30%, between 25% to 65%, between 35% to 60%, or between 40% to 55% at the region that is complimentary to the 20-nt guide sequence of the Cas12a crRNA.

In some embodiments, the target DNA comprises a ZT content between 35% to 50%, between 35% to 55%, between 35% to 60%, between 35% to 65%, between 35% to 70%, between 35% to 75%, between 35% to 85%, or between 35% to 90%, between 35% to 95%, between 45% to 50%, between 45% to 55%, between 45% to 60%, between 45% to 65%, between 45% to 70%, between 45% to 75%, between 45% to 85%, or between 45% to 90%, or between 45% to 95% at the region that is complimentary to the 20-nt guide sequence of the Cas12a crRNA.

In some embodiments, methods described herein modifies the target DNA by introducing an insertion, a deletion, and/or a substitution of the target DNA sequence.

### CRISPR-Cas9 Systems

In some aspects, provided herein is a composition or kit, comprising: (a) a Cas9 protein or a nucleic acid encoding the Cas9 protein; (b) a Cas9 crRNA comprising at least one Z base; and (c) a Cas9 tracrRNA.

In some aspects, provided herein is a kit, comprising: (a) a Cas9 protein or a nucleic acid encoding the Cas9 protein; (b) a Cas9 crRNA comprising at least one Z base; and (c) a Cas9 tracrRNA.

In some aspects, provided herein is a method of cleaving or modifying a target DNA, comprising: contacting the target DNA or a cell comprising the target DNA with: (a) a Cas9 protein, or a nucleic acid encoding the Cas9 protein; (b) a Cas9 crRNA comprising at least one Z base, and (c) a Cas9 tracrRNA; wherein the Cas9 protein, the Cas9 crRNA, and the Cas9 tracrRNA form a complex that cleaves or modifies the target DNA. In some embodiments, the target DNA is a plasmid DNA. In some embodiments, the target DNA comprises at least one Z base.

In some embodiments, the Cas9 crRNA comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 Z bases. In some embodiments, the Cas9 crRNA comprises 1, 2, 3, 4, 5, 6, 7, 8, 9, 10, 11, 12, 13, 14, 15, 16, 17, 18, 19, 20, 21, 22, or 23 Z bases. In some embodiments, the Cas9 crRNA does not comprise an A base.

In some embodiments, the Cas9 crRNA comprises at least one (e.g., 1, 2, 3, 4, 5, 6, 7, 8, 9, 10, 11, 12, 13, 14, 15, 16, 17, 18, 19, or 20) Z base in the 20-nt guide sequence.

In some embodiments, the Cas9 crRNA comprises a 20-nt guide sequence complimentary to a target DNA site with an A or Z content from 5% to 95%, between 5% to 60%, between 5% to 50%, between 10% to 90%, between 10% to 40%, between 15% to 75%, between 15% to 35%, between 20% to 70%, between 20% to 30%, between 25% to 65%, between 35% to 60%, or between 40% to 55%.

In some embodiments, the Cas9 crRNA comprises a 20-nt guide sequence complimentary to a target DNA site with an AT or ZT content between 35% to 50%, between 35% to 55%, between 35% to 60%, between 35% to 65%, between 35% to 70%, between 35% to 75%, between 35% to 85%, or between 35% to 90%, between 35% to 95%, between 45% to 50%, between 45% to 55%, between 45% to 65%, between 45% to 70%, between 45% to 75%, between 45% to 85%, or between 45% to 90%, or between 45% to 95%.

In some embodiments, Cas9 tracrRNA comprises at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or 29) Z base. In some embodiments, the Cas9 tracrRNA does not comprise an A base.

In some aspects, provided herein is a method of cleaving or modifying a target DNA comprising at least one Z base, comprising: contacting the target DNA with: (a) a Cas9 protein or a nucleic acid encoding the Cas9 protein; and (b) a Cas9 guide RNA (gRNA) or a nucleic acid encoding a Cas9 gRNA, wherein the Cas9 protein and the Cas9 gRNA form a complex that cleaves or modifies the target DNA comprising at least one Z base. In some embodiments, the target DNA comprises a Z base in a spacer region or a PAM region or both. In some embodiments, the target DNA is a plasmid DNA.

In some embodiments, the target DNA is the genomic DNA. In some embodiments, the target DNA is a plasmid DNA. In some embodiments, the target DNA comprises at least one Z base at the region that is complimentary to the 20-nt guide sequence of the Cas9 crRNA. In some embodiments, the target DNA comprises at least one (e.g., 1, 2, 3, 4, 5, 6, 7, 8, 9, 10, 11, 12, 13, 14, 15, 16, 17, 18, 19, or 20) Z base at the region that is complimentary to the 20-nt guide sequence of the Cas9 crRNA.

In some embodiments, the target DNA comprises a Z content between 5% to 95%, between 5% to 60%, between 5% to 50%, between 10% to 90%, between 10% to 40%, between 15% to 75%, between 15% to 35%, between 20% to 70%, between 20% to 30%, between 25% to 65%, between 35% to 60%, or between 40% to 55% at the region that is complimentary to the 20-nt guide sequence of the Cas9 crRNA.

In some embodiments, the target DNA comprises a ZT content between 35% to 50%, between 35% to 55%, between 35% to 60%, between 35% to 65%, between 35% to 70%, between 35% to 75%, between 35% to 85%, or between 35% to 90%, between 35% to 95%, between 45% to 50%, between 45% to 55%, between 45% to 60%, between 45% to 65%, between 45% to 70%, between 45% to 75%, between 45% to 85%, or between 45% to 90%, or between 45% to 95% at the region that is complimentary to the 20-nt guide sequence of the Cas9 crRNA.

In some embodiments, methods described herein modifies the target DNA by introducing an insertion, a deletion, and/or a substitution of the target DNA sequence.

### Base Editor

In some aspects, provided herein is a composition comprising: (a) a Cas9-guided base editor or a nucleic acid encoding the Cas9-guided base editor; (b) a Cas9 crRNA comprising at least one Z base; and (c) a Cas9 tracrRNA.

In some aspects, provided herein is a kit comprising: (a) a Cas9-guided base editor or a nucleic acid encoding the Cas9-guided base editor; (b) a Cas9 crRNA comprising at least one Z base; and (c) a Cas9 tracrRNA.

In some aspects, provided herein is a method of modifying a target DNA comprising: contacting the target DNA or a cell comprising the target DNA with: (a) a Cas9-guided base editor or a nucleic acid encoding the Cas9-guided base editor; (b) a Cas9 crRNA comprising at least one Z base; and (c) a Cas9 tracrRNA, wherein the Cas9-guided base editor, the Cas9 crRNA, and the Cas9 tracrRNA form a complex that induces a base change of the target DNA.

In some embodiments, the Cas9-guided base editor is an adenine base editor (ABE). In some embodiments, the Cas9-guided base editor is ABE8e.

In some embodiments, the Cas9-guided base editor, the Cas9 crRNA, and the Cas9 tracrRNA form a complex that induces a A-to-G change of the target DNA. In some embodiments, the Cas9-guided base editor, the Cas9 crRNA, and the Cas9 tracrRNA form a complex that induces A-to-G changes with a frequency of at least 5%, at least 10%, at least 15%, at least 20%, at least 25%, at least 30%, at least 35%, at least 40%, at least 45%, at least 50%, at least 55%, or at least 60%.

In some embodiments, the Cas9 crRNA comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 Z bases. In some embodiments, the Cas9 crRNA comprises 1, 2, 3, 4, 5, 6, 7, 8, 9, 10, 11, 12, 13, 14, 15, 16, 17, 18, 19, 20, 21, 22, or 23 Z bases. In some embodiments, the Cas9 crRNA does not comprise an A base.

In some embodiments, the Cas9 crRNA comprises at least one (e.g., 1, 2, 3, 4, 5, 6, 7, 8, 9, 10, 11, 12, 13, 14, 15, 16, 17, 18, 19, or 20) Z base in the 20-nt guide sequence. In some embodiments, the cell is a mammalian cell.

### EXAMPLES

### Example 1: Materials and methods for Examples 2-13

### Materials

All reagents are from New England Biolabs, unless otherwise stated.

### Cell Culture and Transfection

HEK293 cells were cultivated in DMEM (Sigma, D6429) supplemented with 10% FBS and 1% Gibco™ Penicillin-Streptomycin (10,000 U/mL) at 37° C. and 5% CO₂. A day before transfection, HEK293T cells were seeded into 24-well cell culture plates at a density of 50,000 cells per well. The transfection mixtures were prepared by mixing 200 ng mRNA or DNA with 1.8 μl Lipofectamine™ 2000 (Invitrogen, 11668027) in 100 μL serum-free Opti-MEM.

### Construction of Plasmid

pMRNA-eGFP and pMRNA-Fluc plasmids were constructed as described previously (81). All artificial DNA sequences were synthesized by Integrated DNA Technologies, IDT (San Diego, US). Plasmid pCMV-GFP was received from Dr. Connie Cepko (82). Fragment of Fluc were amplified from pMRNA-Fluc and digested with Agel/NotI following by inserting in pCMV-GFP to generate the pCMV-Fluc plasmid.

E. coli and Yeast Transformation

E. coli One Shot™ MAX Efficiency™ DH5α-TIR Competent Cells (Fisher, 12297016) was used to harvest plasmids used in this study. Plasmids were extracted and purified using QIAprep Spin Miniprep Kit (Qiagen, 27104). Yeast transformation were performed as described previously (83) using Frozen-EZ Yeast Transformation II Kit (ZYMO RESEARCH, T2001). Cells were harvested by centrifugation at 13,000×g for 2 minutes (min) after outgrowth for 3 hours (hr) at 30° C. and 230 rpm. Cells were then suspended using PBS (pH=7.2) for flow cytometry analysis.

### DNA Amplification

PCR products were generated by NEB Next High-Fidelity 2X PCR Master Mix (NEB, M0541), unless otherwise stated. All oligonucleotides were synthesized by Integrated DNA Technologies (IDT). Taq DNA Polymerase (NEB, M0320L) was used to make DNA fragments containing dATP or dZTP. Reactions were performed as per the manufacturer's protocol with 50 μl volume. For dZTP-DNA, 100 mM dATP was replaced with 100 mM dZTP (Trilink, N-2003-1). The following PCR program was performed on a thermal cycler (Applied Biosystems™): step 1, 98° C. for 30 seconds(s); step 2, 98° C. for 30 s; step 3, 60° C. for 30 s; step 4, 68° C. for 1 min; step 5, repeat steps 2-4 for a total of 33 cycles; step 6, 68° C. for 5 min; step 7, 4° C. for 10 min. The target products were purified using a Monarch Gel Purification Kit (NEB, T1020S). DNA concentrations were the measured using a NANODROP ONE (Thermo Scientific). Phusion® High-Fidelity DNA Polymerase (NEB, M0530S) was tested in screening of DNA polymerase. When plasmids were used as PCR templates, the resulting PCR products were treated with DpnI restriction enzyme (NEB, R0176S) to degrade templates. Primers were shown in Table 18.

TABLE 18
Primers used for PCR test in this study.

| PRIMER NAME | 5′ > 3′ SEQUENCE |
|---|---|
| G001 | GATCCACCGGTCGCCACCATG |
| G002 | GTGTTCTGCTGGTAGTGGTC |
| G030 | GGGTGTCGGGGCTGGCTTA |
| G031 | CAATTTCACACAGGAAACAGC |
| G050 | GCGAATTAATACGACTCACTATAGGGCTTAAGTATAAGGAGGAAAAAATATGGTGAGC |
| | AAGGGCGAGGAGCT |
| G051 | AAACCCCTCCGTTTAGAGAGGGGTTATGCTAGTTATTACTTGTACAGCTCGTCCAT |
| G052 | GCGAATTAATACGACTCACTATAGGGCTTAAGTATAAGGAGGAAAAAATATGATCAGT |
| | CTGATTGCGGC |
| G053 | AAACCCCTCCGTTTAGAGAGGGGTTATGCTAGTTATTACCGCCGCTCCAGAATC |
| G063 | GCGAATTAATACGACTCACTATAGGGCTTAAGTATAAGGAGGAAAAAATATGCACCAC |
| | CACCACCACCAC GTGTCTAAGGGAGAAGAACT |
| G064 | AAACCCCTCCGTTTAGAGAGGGGTTATGCTAGTTATCACTTATATAATTCGTCCATACC |
| | A |
| G097 | TGTAAAACGACGGCCAGT |
| G098 | CAGGAAACAGCTATGACC |
| G103 | ACACTCTTTCCCTACACGACGCTCTTCCGATCTGGAGGACAAAGTACAAACGGC |
| G104 | GACTGGAGTTCAGACGTGTGCTCTTCCGATCTATCGATGTCCTCCCCATTGG |
| G126 | TTGGACCCTCGTACAGAAGCTA |
| G127 | TTCTTCCTACTCAGGCTTTATTCAA |
| G132 | CCACCTGGGTCGACATTGAT |
| G133 | TGTCCTTCCGAGTGAGAGAC |
| G111 | ATGGTGAGCAAGGGCGAGGAG |
| G112 | TTACTTGTACAGCTCGTCCATGCC |
| G126 | TTGGACCCTCGTACAGAAGCTA |
| G127 | TTCTTCCTACTCAGGCTTTATTCAA |
| G103 | ACACTCTTTCCCTACACGACGCTCTTCCGATCTGGAGGACAAAGTACAAACGGC |
| G104 | GACTGGAGTTCAGACGTGTGCTCTTCCGATCTATCGATGTCCTCCCCATTGG |
| G105 | CCATCCCCTTCTGTGAATGT |
| G106 | GGAGATTGGAGACACGGAGA |
| G248 | GCTGACCCTGAAGTTC |
| G248-A- | GAACTTCAGGGTCAGC |
| COMPLEMENT | |
| G248-Z- | GZZCTTCZGGGTCZGC |
| COMPLEMENT | |
| G250 | GACGGCAGCGTGCAGCTC |
| G250-A- | GAGCTGCACGCTGCCGTC |
| COMPLEMENT | |
| G250-Z- | GZGCTGCZCGCTGCCGTC |
| COMPLEMENT | |
| G132 | CCACCTGGGTCGACATTGAT |
| G133 | TGTCCTTCCGAGTGAGAGAC |
| G138 | GCGGGCCCGGGATCCACCGGTATGGAAGACGCCAAAAACATAAAG |
| G139 | CAGCCTGCACCTGAGGAGTGCGGCCGCTTACACGGCGATCTTTCCGC |
| EGFP-R | GTCTTGTAGTTGCCGTCGTC |

### DNA Melting Curve Analysis

SYBR™ Green I Nucleic Acid Gel Stain (Fisher, S7563) was diluted 1:10,000 in 1X TE pH 8.5 to generate the reaction buffer. 20 μl volume of reaction buffer contained 20 ng double stranded DNA (dsDNA) was added into a 96-well qPCR plate (Bio-Rad, HSP9631). The mixture was stained for 30 min at room temperature. A high-resolution melting curve program was carried out by thermocycling on CFX96 Touch Real-Time PCR Detection System (Bio-Rad). The following program was used: 25° C. for 10 s, melting curve 20.0° C. to 95° C. for 5 s at 0.2° C. or 0.5° C. increments.

cDNA Synthesis

cDNA synthesis were performed using ProtoScript II First Strand cDNA Synthesis Kit (NEB, E6560) with 200 ng ATP-mRNA or 600 ng ZTP-mRNA in a total reaction volume of 20 μl. Reactions were incubated for 2 hr at 42° C. 2 μL cDNA was added to 50 μL NEBNext High-Fidelity 2X PCR Master Mix (NEB, M0541S) containing specific primers G111 and G112. The target products were purified using a Monarch Gel Purification Kit (NEB, T1020S).

### Illumina MiSeq Library Preparation, Sequencing and Computational Methods for Determining Error Rates

The DNA amplicon library was prepared following the manufacturer's recommendations. Sequencing was carried out Illumina MiSeq instrument. The DNA amplicon library for sequencing was quantified using the Qubit 2.0 Fluorometer (Life Technologies, Carlsbad, CA, USA). NEBNext® Ultra™ DNA Library Prep Kit for Illumina (New England Biolabs, Ipswich, MA, USA), clustering, and sequencing reagents were used throughout the process following the manufacturer's recommendations. Briefly, the genomic DNA was fragmented by acoustic shearing with a Covaris LE220 instrument. Fragmented DNA was end-repaired and adenylated. Adapters were ligated after adenylation of the 3′ ends followed by enrichment by limited cycle PCR. The DNA library was validated using D1000 ScreenTape on the Agilent 4200 TapeStation (Agilent Technologies, Palo Alto, CA, USA), and was quantified using a Qubit 2.0 Fluorometer. The sample was sequenced using a 2×150 paired-end (PE) configuration. Image analysis and base calling were conducted by the MiSeq Control Software (MCS) on the MiSeq instrument. Raw sequencing data (.bcl files) generated from Illumina MiSeq was converted into fastq files and de-multiplexed using Illumina's bcl2fastq software. One mismatch was allowed for index sequence identification. Fastq files were trimmed with Trimmomatic-0.36, then mapped using BWA mem. The mpileup file was generated from mapped files using samtools mpileup. Subsequently, variants were called using Varscan-2.3.9 mpileup2cns with the following criteria: --min-coverage 10,--min-reads2 4,--min-var-freq 0.005,--p-value 0.05,--strand-filter 1. A tabulated summary of bases aligned per site was generated by parsing the output from samtools mpileup on the BWA-aligned bam files.

In vitro DNA Cleavage Assays

100 ng dATP or dZTP containing dsDNA PCR products that were purified using a Monarch Gel Purification Kit were subjected to restriction endonuclease digestion in a 20 μl reaction mixture following the manufacture's protocol. The following restriction endonucleases were used: BsrFI (NEB, R0739S), BstYI (NEB, R0523S), BsrI (NEB, R0527S), FauI (NEB, R0651S), EcoRI (NEB, R0101S). Reactions were terminated by adding 5 μL Gel Loading Dye, Purple (6X) and analyzed by agarose gel electrophoresis. Imaging was carried out on G:BOX system (Syngene, USA). For in vitro digestion of DNA with Streptococcus pyogenes Cas9 nuclease (NEB, M0386) and Lachnospiraceae bacterium Cpf1 nuclease (NEB, M0653S), plasmid p MRNA-GFP and PCR fragments amplified from p MRNA-GFP using primers G050/G051 were used as DNA substrate. The in vitro reaction was performed as per the manufacture's protocol. The final mixture was loaded into an agarose gel for analysis.

RNA Synthesis by in vitro Transcription

All RNA used in study was produced using the HiScribe™ T7 High Yield RNA Synthesis Kit (NEB, E2040S). For sgRNA, tracrRNA and crRNA transcription, 75 ng synthetic Ultramer™ DNA Oligonucleotides (IDT) were used as templates (Table 19). dsDNA templates were prepared in a 50 μL reaction containing 100 ng pMRNA-GFP plasmid, 25 μL NEBNext High-Fidelity 2X PCR Master Mix, and 5 μL Tail primer mix (SBI, MR-TAIL-PR). PCR products were cleaned using Monarch Gel Purification Kit. Templates were transcribed using the HiScribe™ T7 High Yield RNA Synthesis Kit (NEB, E2040S) and the standard RNA synthesis protocol in a total volume of 20 μl for 2 hr at 37° C. For unmodified mRNA, 40 mM m7G(5′)ppp(5′) G RNA Cap Structure Analog (NEB, S1404), clean cap GG (Trilink, N-7133-1), and 100 mM each of ATP, UTP, GTP, and CTPs were used. For modified mRNA synthesis, the equivalent ATP was replaced with 100 mM ZTP (Trilink, N-1001-5, 2-Aminoadenosine-5′-Triphosphate). After transcription, 20 μL of RNA reaction was added to 4 μl Nuclease-free water, 1 μl Antarctic Phosphatase (NEB, M0289), 3 μl Antarctic Phosphatase Reaction Buffer (NEB, B0289SVIAL), and 2 μl DNase (Invitrogen, AM2238). The total 30 μl mixture was incubated at 37° C. for 30 min. RNA was purified using a Monarch RNA Cleanup Kit (NEB, T2040) or MEGAclear™ Transcription Clean-Up kit (Invitrogen, AM1908). RNA quantity and quality was detected by NanoDrop. For mRNA transcription, 0.2-0.8 μg PCR products were used as templates. For sgRNA, tracrRNA and crRNA transcription, 75 ng synthetic Ultramer™ DNA Oligonucleotides (IDT) were used as templates (Table 19). The RNA fragments were loaded on 15% TBE-Urea gels (Thermo Fisher, EC6885BOX) or 2% agarose TAE gels for extraction and purification if further purification was needed. RNA was recovered from gels using Zymoclean Gel RNA Recovery Kit (ZYMO, R1011).

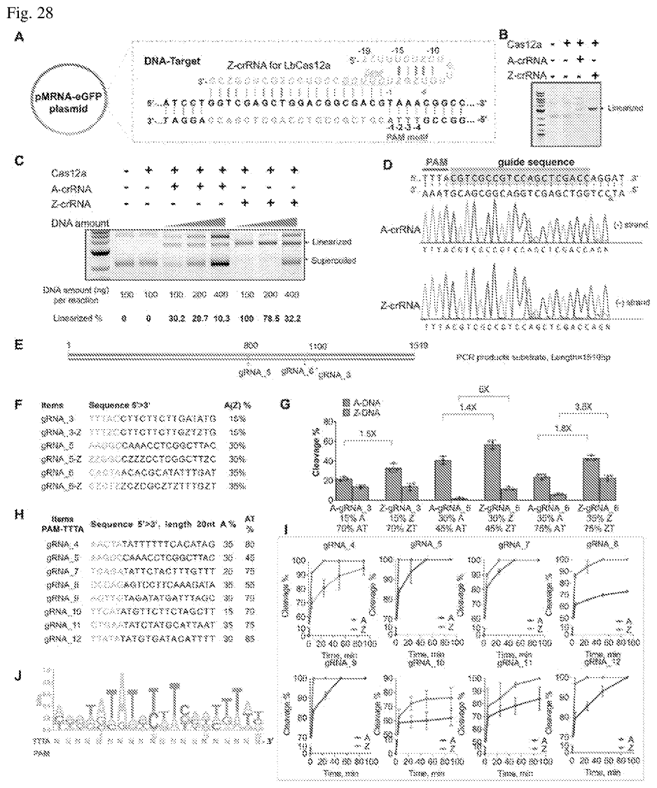

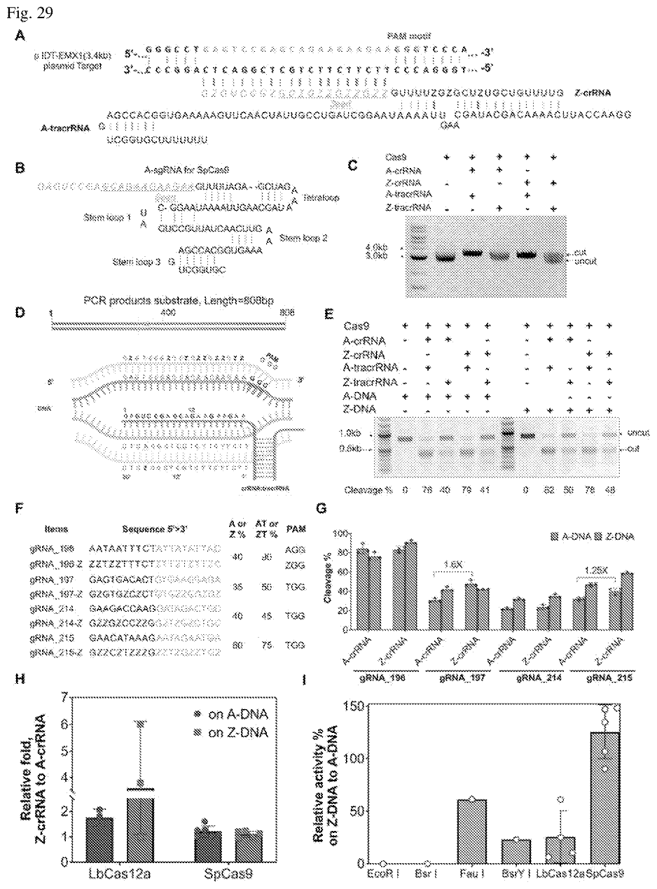

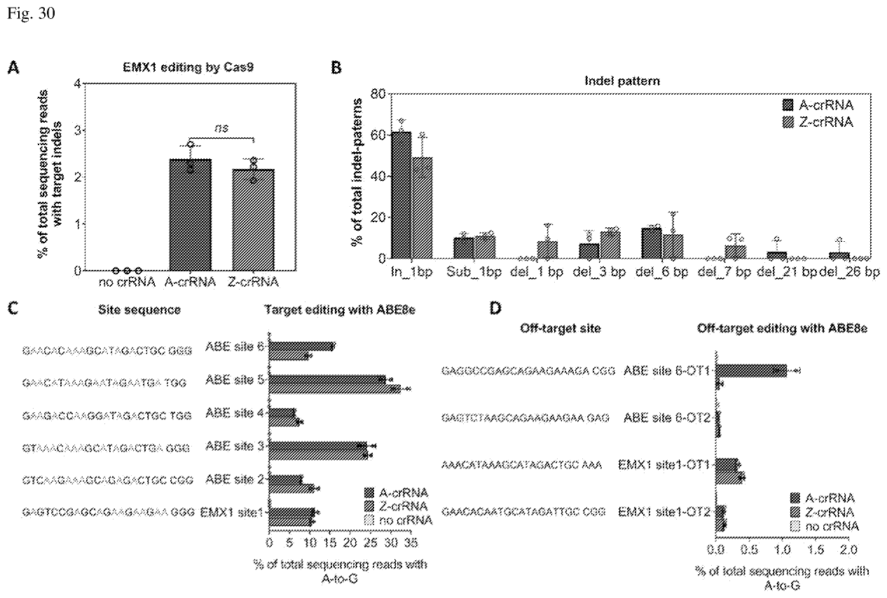

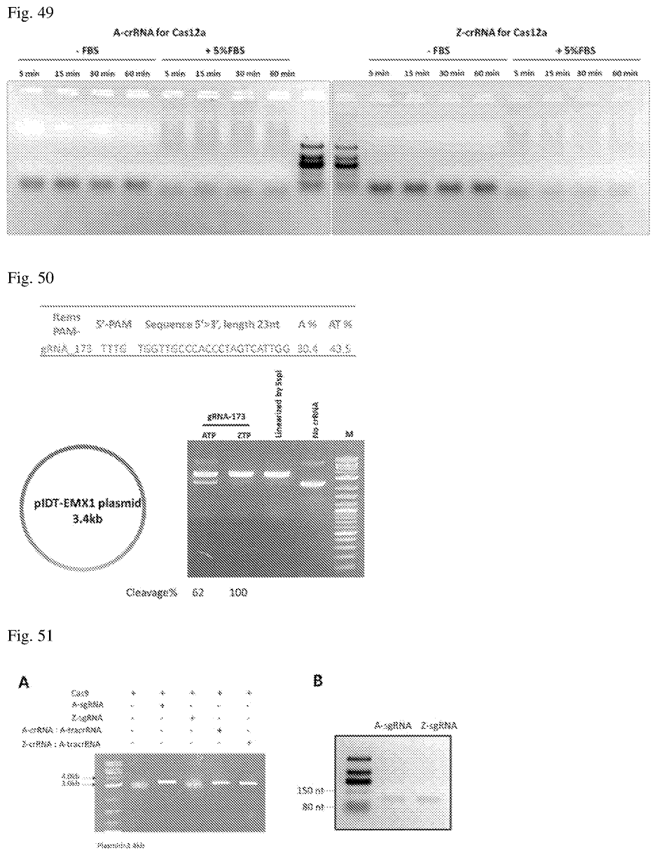

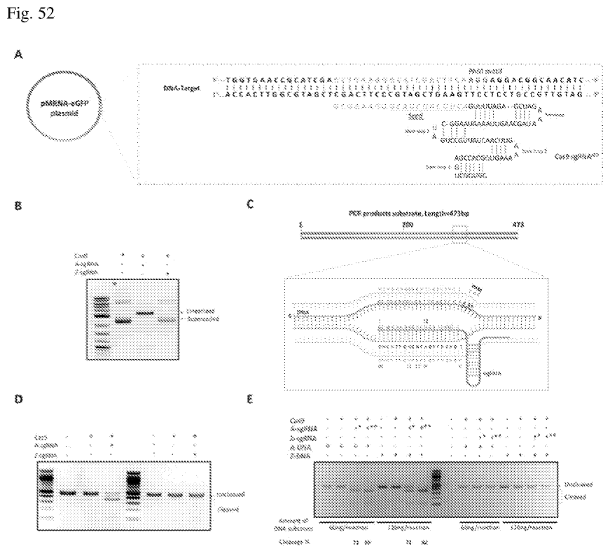

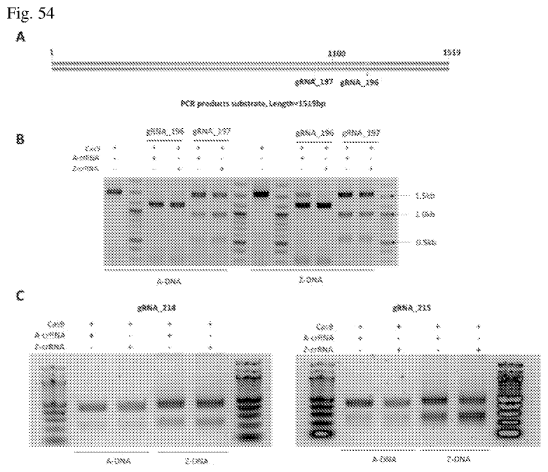

TABLE 19
DNA duplex used for sgRNA, crRNA and tracrRNA transcription.

| Name | Sequence 5′ > 3′ | For usage |
|---|---|---|
| gRNA_1 | TAATACGACTCACTATAGGCTGAAGGGCATCGACTTCAGTTTTAGAGCTAGAAATAGC | FIG. 52 |
| | AAGTTAAAATAAGGCTAGTCCGTTATCAACTTGAAAAAGTGGCACCGAGTCGGTGC | |
| gRNA_2 | TAATACGACTCACTATAGGAATTTCTACTGTTGTAGATCGTCGCCGTCCAGCTCGACC | FIG. 28A-D |
| gRNA_3 | TAATACGACTCACTATAGGAATTTCTACTGTTGTAGATTTTACCTTCTTCTTGATATG | FIG. 28F-G |
| gRNA_4 | TAATACGACTCACTATAGGAATTTCTACTGTTGTAGATAACTATATTTTTTCACATAG | |
| gRNA_5 | TAATACGACTCACTATAGGAATTTCTACTGTTGTAGATAAGGCCAAACCTCGGCTTAC | |
| gRNA_6 | TAATACGACTCACTATAGGAATTTCTACTGTTGTAGATCACTAACACGCATATTTGAT | |
| gRNA_7 | TAATACGACTCACTATAGGAATTTCTACTGTTGTAGATTCAGATATTCTACTTTGTTT | |
| gRNA_8 | TAATACGACTCACTATAGGAATTTCTACTGTTGTAGATCCCAGAGTCCTTCAAAGATA | |
| gRNA_9 | TAATACGACTCACTATAGGAATTTCTACTGTTGTAGATAGTTGTAGATATGATTTAGC | |
| gRNA_10 | TAATACGACTCACTATAGGAATTTCTACTGTTGTAGATTTCATATGTTCTTCTAGCTT | |
| gRNA_11 | TAATACGACTCACTATAGGAATTTCTACTGTTGTAGATCTGAATATCTATGCATTAAT | |
| gRNA_12 | TAATACGACTCACTATAGGAATTTCTACTGTTGTAGATTTATATATGTGATACATTTT | |
| gRNA_28 | TAATACGACTCACTATAGGAATTTCTACTGTTGTAGATGAATCAACAAAAAAATTAGT | FIG. 49 |
| gRNA_29 | TAATACGACTCACTATAGGAATTTCTACTGTTGTAGATAGATTTAAAAAGAATAAGTA | |
| gRNA_30 | TAATACGACTCACTATAGGAATTTCTACTGTTGTAGATAAAGAGCAACAAAATGGCTT | |
| gRNA_173 | TAATACGACTCACTATAGGAATTTCTACTGTTGTAGAT TGGTTGCCCACCCTAGTCAT TGG | FIG. 50 |
| gRNA_182 | TAATACGACTCACTATAGG GAGTCCGAGCAGAAGAAGAA | EMX1 site1 |
| | GTTTTAGAGCTATGCTGTTTTG | FIG. 29A, |
| | | C, D, E |
| | | FIG. 30 |
| gRNA_183 | TAATACGACTCACTATAGGGGAACCATTCAAAACAGCATAGCAAGTTAAAATAAGGCTAG | tracrRNA |
| | TC | of SpCas9 |
| | CGTTATCAACTTGAAAAAGTGGCACCGAGTCGGTGCTTTTTTT | |
| gRNA_202 | TAATACGACTCACTATAGG GAGTCCGAGCAGAAGAAGAA | FIG. 29B |
| | GTTTTAGAGCTAGAAATAGCAAGTTAAAATAAGGCTAGTCCGTTATCAACTTGAAAAAGT | FIG. 51 |
| | G | |
| | GCACCGAGTCGGTGC | |
| gRNA_196 | TAATACGACTCACTATAGG AATAATTTCTATTATATTACGTTTTAGAGCTATGCTGTTTTG | FIG. 29F, G |
| gRNA_197 | TAATACGACTCACTATAGG GAGTGACACTGTGAAGGAGA | FIG. 54, 55 |
| | GTTTTAGAGCTATGCTGTTTTG | |
| gRNA_ABE | TAATACGACTCACTATAGG GTCAAGAAAGCAGAGACTGC | |
| site 2 | GTTTTAGAGCTATGCTGTTTTG | |
| gRNA_ABE | TAATACGACTCACTATAGG GTAAACAAAGCATAGACTGA | |
| site 3 | GTTTTAGAGCTATGCTGTTTTG | |
| gRNA_ABE | TAATACGACTCACTATAGG GAAGACCAAGGATAGACTGC | =gRNA_214 |
| site 4 | GTTTTAGAGCTATGCTGTTTTG | |
| gRNA_ABE | TAATACGACTCACTATAGG GAACATAAAGAATAGAATGA | =gRNA_215 |
| site 5 | GTTTTAGAGCTATGCTGTTTTG | |
| gRNA_ABE | TAATACGACTCACTATAGG GAACACAAAGCATAGACTGC | |
| site 6 | GTTTTAGAGCTATGCTGTTTTG | |

Flow cytometry Detection

Adherent cells were washed with 250 μL PBS (pH=7.2). Next, 250 μL of 0.25% Trypsin-EDTA (Gibco, 25200056) was added into the well and cells were incubated for 5 min at 37° C. to completely detach cells. 750 μL of DMEM was used to stop trypsin digestion. Samples were applied to the BD Accuri™ C6 Flow Cytometer (BD Biosciences) directly, and GFP fluorescence was measured. Data was analyzed using FlowJo software.

### Firefly Luciferase Assay

A day before transfection, HEK293T cells were seeded into 24-well cell culture plates at a density of 70,000 cells per well and transfected with 200 ng DNA. After 72 h post transfection, cell pellets were lysed to quantify luciferase expression with a Luciferase Assay System (Promega, E1500) and a Varioskan LUX Multimode Microplate Reader (ThermoFisher, US).

### Cell-Free Translation and Protein Assay

In vitro translations were carried out using a PURExpress In Vitro Protein Synthesis Kit (NEB, E6800) following the manufacture's protocol. 250 ng plasmid DNA template or PCR products were used in a 25 μL reaction. The reaction was incubated at 37° C. for 4 hr. 5 μL of sample was loaded into 10-20% Tris-Glycine Mini Protein Gels (Invitrogen, XP10200BOX) to be examined and visualized by Coomassie staining.

Poly(Z) tailing of RNA

5 μg RNA, 1 mM ATP or ZTP, 1X Reaction Buffer, 40U RNase Inhibitor (NEB, M0314L) and 4U E. coli Poly(A) Polymerase (NEB, M0276L) was mixed in a 20 μL reaction volume. Reaction of tailing A was incubated at 37° C. for 0.5 hr, whereas tailing Z was incubated at 37° C. for 2 hr. RNA was purified using a Monarch RNA Cleanup Kit (NEB, T2040).

In Vitro Cleavage Activity Assay with Different crRNA or sgRNA

The following assay was modified from Hirano et al. (84). Plasmid pIDT-DNA4 linearized by SspI restriction endonuclease was used as a DNA substrate. A 27 μl mixture containing 1.5 μmol LbCas12a, 2.5 μmol crRNA, and 1x buffer r2.1, was incubated at 25° C. for 10 min. Subsequently, 300 ng of DNA substrate in 3 μl was added to the initial reaction. For Cas9, 2 μg of crRNA and 4 μg tracrRNA were mixed in 20 μl RNase free water. The RNA mixture was incubated for 5 min at 95° C. and cool down to room temperature. A 27 μ 1 mixture containing 1.5 μmol SpCas9, 3 μmol crRNA:tracrRNA, and 1x buffer r3.1, was incubated at 25° C. for 10 min. Subsequently, DNA substrate in 3 μ 1 was added to the initial reaction. 6 μ 1 of sample was collected at 5, 25, 50 and 90 mins and stopped by adding a 2 μ 1 stop buffer containing 0.5% SDS and 40 mM EDTA. Each sample was subsequently analyzed by gel electrophoresis. The cleavage fraction percent was calculated using band intensities by a formula described previously (36).

### Serum Stability Assay of RNA

The following assay was modified from Park et. al. (85). Briefly, 1 μg RNA produced by in vitro transcription was added to 10 μl of PBS containing 5% Fetal Bovine Serum (Sigma, F2442) and the mixture was incubated at 37° C. for 5, 15, 30, or 60 mins. Next, 10 μl of RNA dye (NEB, B0363S) was added and the mixture was heated at 70° C. for 5 min to denature RNA and proteins. RNA samples were loaded into a 2% agarose gel for analysis. Quantitative analysis was obtained by three independent experiments and band intensity quantification was conducted using the GelAnalyzer.

In vitro Cell Genome Editing and NGS Sequencing Analysis.

One day prior to transfection, HEK293T or Hela cells were seeded in a 48-well plate at a cell density of 1-2×10⁴ cells per well. Medium was changed with 199 μl Opti-MEM on the day of transfection. Cas9 mRNA was purchased from Trilink (L-7206-100). ABE8e mRNA was obtained from Dr. David Liu's lab at the Broad Institute of Harvard and MIT. For Cas9 and ABE8e transfection, 250 ng mRNA was added to 25 of Opti-MEM, followed by addition of 250 ng guide RNA. Meanwhile, 1 μl of Lipofectamine RNAiMAX (Invitrogen, 13778075) was diluted into 25 μl of Opti-MEM and then mixed with mRNA/gRNA sample. The mixture was incubated for 15 min prior to addition to the cells. 200 μl of 2xDMEM was added 18 h post lipofection and the cells were incubated for 3 days until editing analysis. Genomic DNA was extracted from transfected cells using DNEasy kit (Qiagen, 69504) following the manufacture's protocol. Targeted regions flanking the on-target or off-targeted sites were amplified using genomic DNA template, specific primers (Table 20) and by Q5® Hot Start High-Fidelity 2X Master Mix (NEB, M0494S). Targeted amplicon sequencing was carried out by Genwiz (Azenta, South Plainfield, NJ, US) with Amplicon-EZ protocol. Amplicon sequencing data were analyzed with CRISPResso2 or BE-Analyzer (86, 87).

TABLE 20
Primers of amplicon PCR used for base editing.

| Primer name | 5′ > 3′ sequence |
|---|---|
| EMX1-site 1, forward | ACACTCTTTCCCTACACGACGCTCTTCCGATCT GCCTCCTGAGTTTCTCATCTGT |
| EMX1-site 1, reverse | GACTGGAGTTCAGACGTGTGCTCTTCCGATCTCCCTAGTCATTGGAGGTGACAT |
| ABE site 2, forward | TCCTCACCTTCAGCAGCTCCTC |
| ABE site 2, reverse | GTGGCAGGCAGATTATCATTCCCATC |
| ABE site 3, forward | CATACTTAAAGTAAAGCAGAAGGAATAACAGTGC |
| ABE site 3, reverse | GAAGTTTCATTCCACTATAGAACTGGTTCTG |
| ABE site 4, forward | GAGTACTCTCCAATGACACCTTTGGC |
| ABE site 4, reverse | ATCATAGCGAGACCCTGTCTCAGC |
| ABE site 5, forward | CAACTAAAGTCACTCTGCTGTGCTATTAGATAC |
| ABE site 5, reverse | CAGAAAGACAAGTTTCACATGTTCTCACTC |
| ABE site 6, forward | ACAAGACCTGGCTGAGCTAACTGTG |
| ABE site 6, reverse | CCAGCCCCATCTGTCAAACTGTG |
| EMX1-site 1, OT1, forward | CACCTGGGCGAGAAAGGTAACTTATG |
| EMX1-site 1, OT1, reverse | CAGTGTTGGCTTTCACAAGGATGC |
| EMX1-site 1, OT2, forward | AGAAGGAGGTGCAGGAGCTAGAC |
| EMX1-site 1, OT2, reverse | CCCTCAGCCACTTTATTTCATCCCC |
| ABE site 6, OT1, forward | GTGTGGAGAGTGAGTAAGCCA |
| ABE site 6, OT1, reverse | ACGGTAGGATGATTTCAGGCA |
| ABE site 6, OT2, forward | CACAAAGCAGTGTAGCTCAGG |
| ABE site 6, OT2, reverse | CTTTTTTGGTACTCGAGTGTTATTCAG |

Statistical analysis and Graphical Illustrations

Curve plotting and statistical analysis were performed using Prism 8 (GraphPad, La Jolla, CA). Data are shown as means+standard error of the mean for groups of two or more replicates or as individual values with the mean indicated. Graphical illustrations were created using BioRender (https://biorender.com/) and ChemDraw.

### Example 2: Z Incorporation Effects DNA Stability

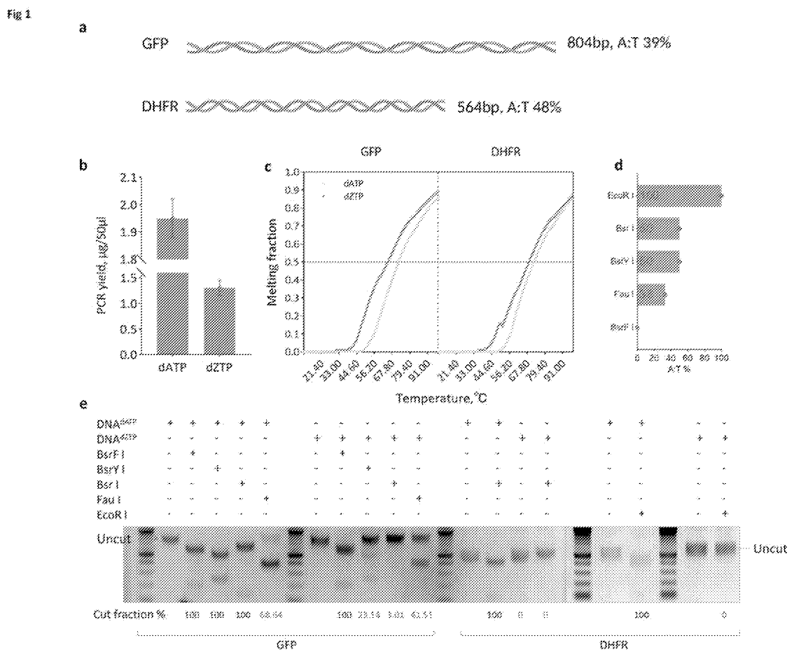

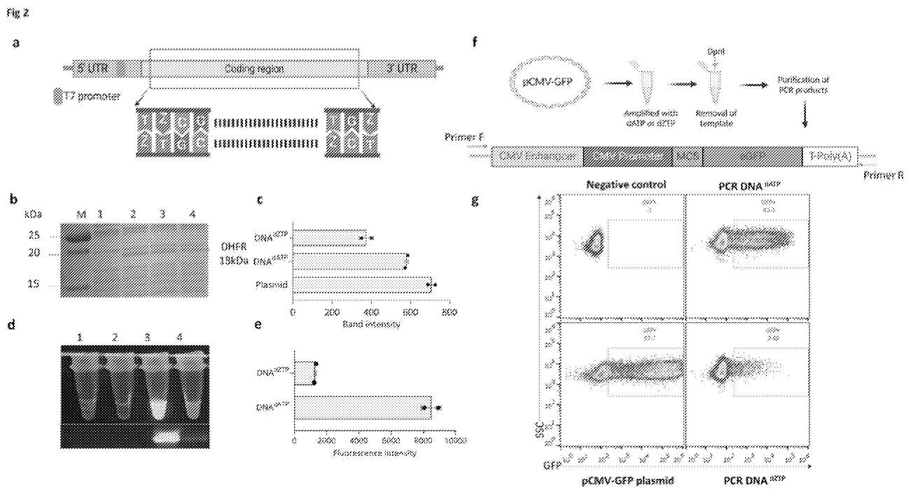

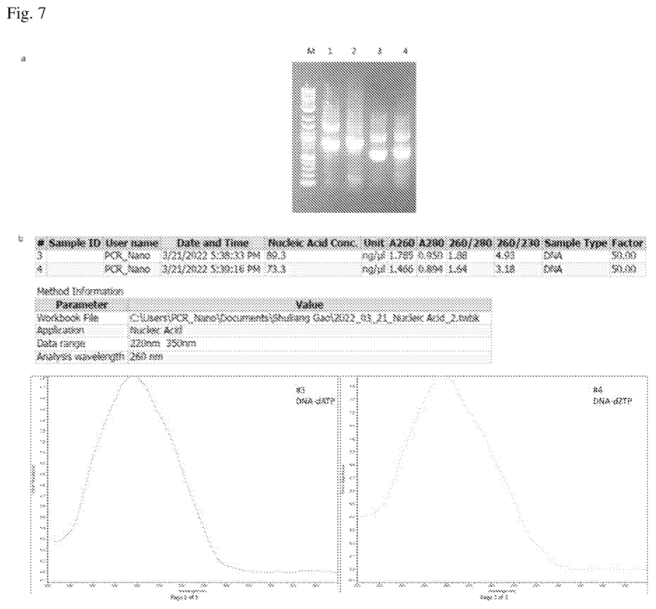

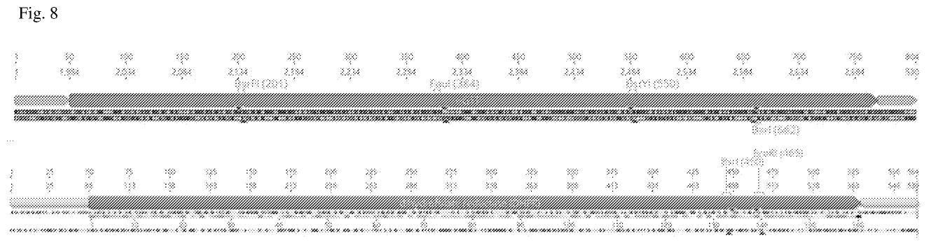

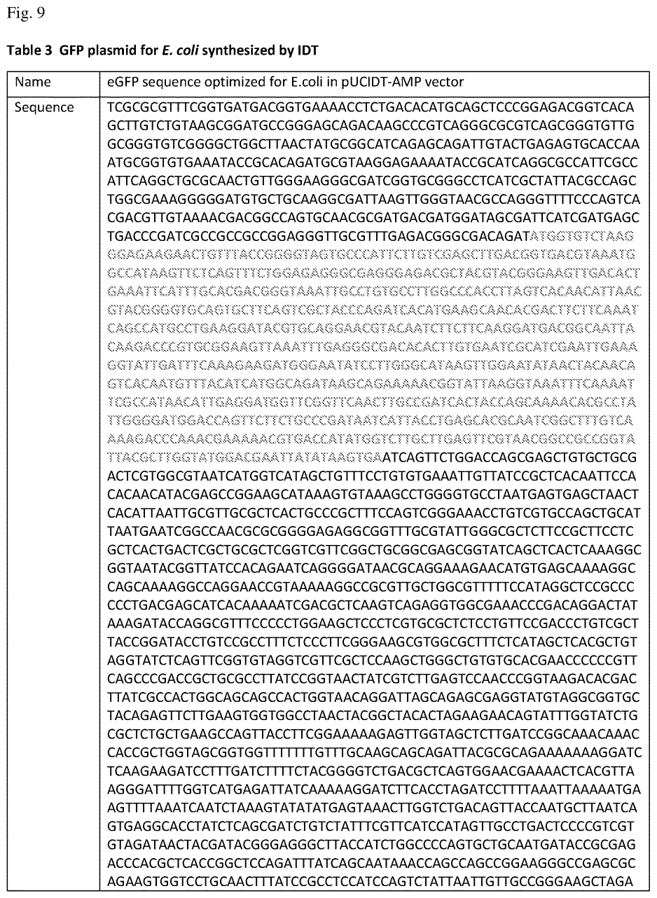

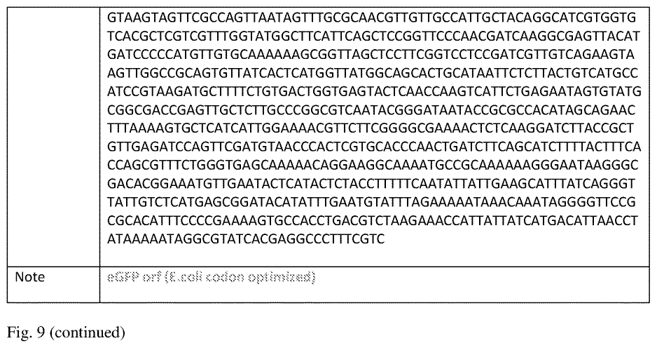

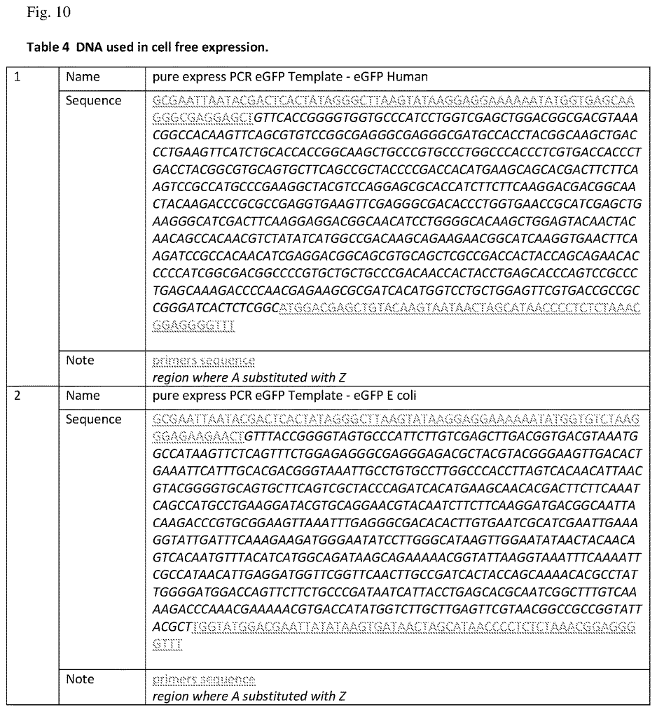

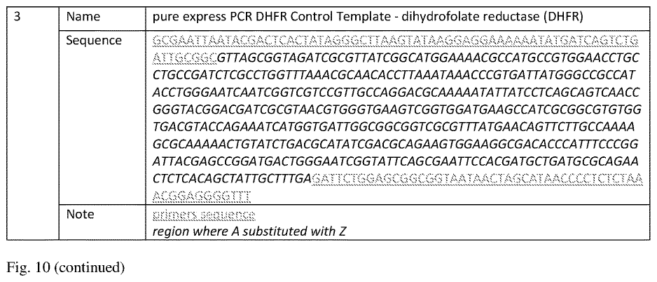

To prepare enough DNA for this study, we firstly screened different DNA polymerases. To our knowledge, both family A DNA polymerase including Taq DNA polymerase and family B DNA polymerase including Pfu polymerase are able to incorporate modified nucleotides into DNA strand (19). We designed two DNA strands to investigate this goal (FIG. 1a, 8, Table 1, Table 2). We screened two DNA polymerases Taq and Pfu, from Thermus aquaticus YT-1 and Pyrococcus furiosus respectively. GFP DNA containing 39% AT nucleotides was selected as the template to PCR-amplify using these two different DNA polymerases. Only Taq succeeded in amplification when dATP was completely replaced with dZTP in PCR reaction volume (unpublished data). This indicates that Taq is more flexible than Phu for dZTP incorporation. Compared to dATP, dZTP decreased 40% yield of PCR products (FIG. 1b), but have little effect on specific-amplification. Compared with A:T, Z:T base pair possesses an extra hydrogen bond and are very similar to a G:C pair in melting temperature and width of the minor groove (23; 24). It has not been published how 100% dZ-dA substitution in longer than 160 bp DNA effect properties. In order to investigate the effect of dZ substitution, we detected melting curves of two DNAs with 39% and 48% AT content respectively. Surprisingly, Tm of both templates decreased after dZTP substituted. For GFP, Tm decreased 13° C. from 75° C. to 62° C. (FIG. 1c). In contrast to increasing melting temperatures of short oligonucleotides with dZTP substitution in previous research (25; 26; 9; 27). Maybe longer DNA strand failed to keep stable natural helices structure because of too much Z:T base pairs (28). On the other hand, DNA^{dZTP} represents different ultraviolet spectra with DNA^{dATP}, ratio of A260/280 decreased drastically for DNA^{dZTP} (FIG. 7a, 7b). We investigated whether these two Z-substituted DNA were resistant to traditional restriction endonucleases (RE) with recognition sites containing one or more As. BsrF I, FauI, BstYI, BsrI and EcoRI with sticky ends containing AT % from 0 to 100% were selected to be evaluated (FIG. 1d, 8). Cleavage assay indicated that resistance to endonucleases were improved 1.47- to 33.3-fold by incorporating Z to DNA strands (FIG. 1e). There were significant positive correlations between the RE resistance and AT % in sticky ends. Maybe both the improved stability with extra H-bond and form changing in groove contribute to RE resistance of Z-substituted DNA strands (5).

TABLE 1
DHFR sequence from NEB

| Name | NEBExpress DHFR-His Control Plasmid |
|---|---|
| Sequence | ATGATCAGTCTGATTGCGGCGTTAGCGGTAGATCGCGTTATCGGCAT |
| | GGAAAACGCCATGCCGTGGAACCTGCCTGCCGATCTCGCCTGGTTTA |
| | AACGCAACACCTTAAATAAACCCGTGATTATGGGCCGCCATACCTGG |
| | GAATCAATCGGTCGTCCGTTGCCAGGACGCAAAAATATTATCCTCAG |
| | CAGTCAACCGGGTACGGACGATCGCGTAACGTGGGTGAAGTCGGTG |
| | GATGAAGCCATCGCGGCGTGTGGTGACGTACCAGAAATCATGGTGAT |
| | TGGCGGCGGTCGCGTTTATGAACAGTTCTTGCCAAAAGCGCAAAAAC |
| | TGTATCTGACGCATATCGACGCAGAAGTGGAAGGCGACACCCATTTC |
| | CCGGATTACGAGCCGGATGACTGGGAATCGGTATTCAGCGAATTCCA |
| | CGATGCTGATGCGCAGAACTCTCACAGCTATTGCTTTGAGATTCTGG |
| | AGCGGCGGTAA |
| Note | Optimized from E. coli codon |

### Example 3: Z-Substituted DNA Element Used for Protein Expression In Vitro and In Vivo

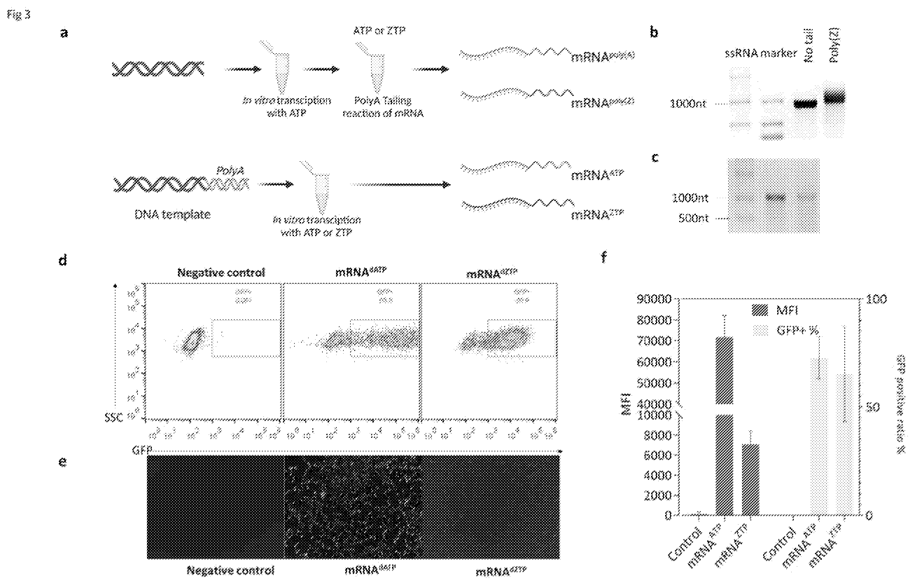

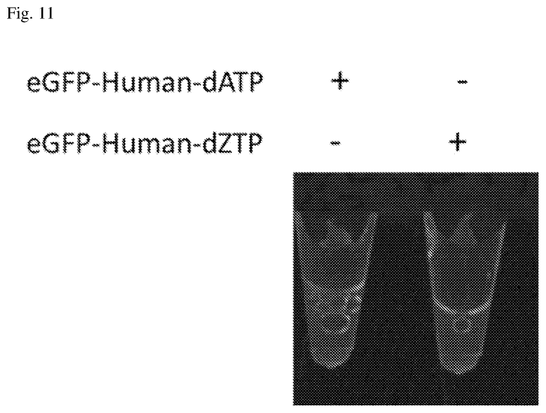

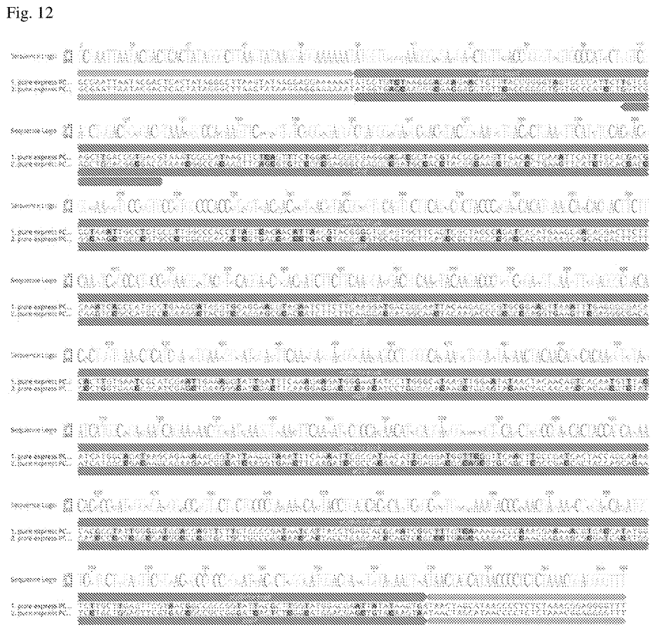

We next sought to evaluate whether the Z-substituted DNA could be used as the template to express specific protein. We tested two proteins, DHFR with 18 kDa molecular wight (MW) and GFP with 27 kDa MW, in cell free system extracted from E. coli using DNA templates with partial Z-substituted coding sequence (FIG. 2a, 8, Table 3, Table 4). Two coding sequences were optimized based on codon usage frequency in E. coli and under control of T7 promotor. When reaction finished, both two proteins were detected, however protein yield decreased by 35.4% and 84.9% for the smaller DHFR and the larger GFP. When the GFP coding sequence was optimized from Human codon usage and used as the template with or without Z-substitutions to do protein expression, fluorescence could not be detected (FIG. 11). Compared to E. coli, Human prefers GC rich codons (FIG. 12) (31). Specificity of codon and anti-codon couldn't be affected by introduction of Z-substitution.

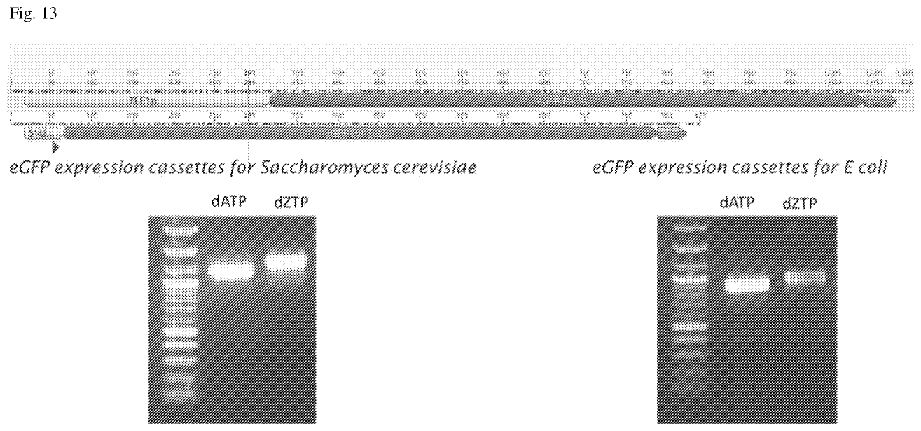

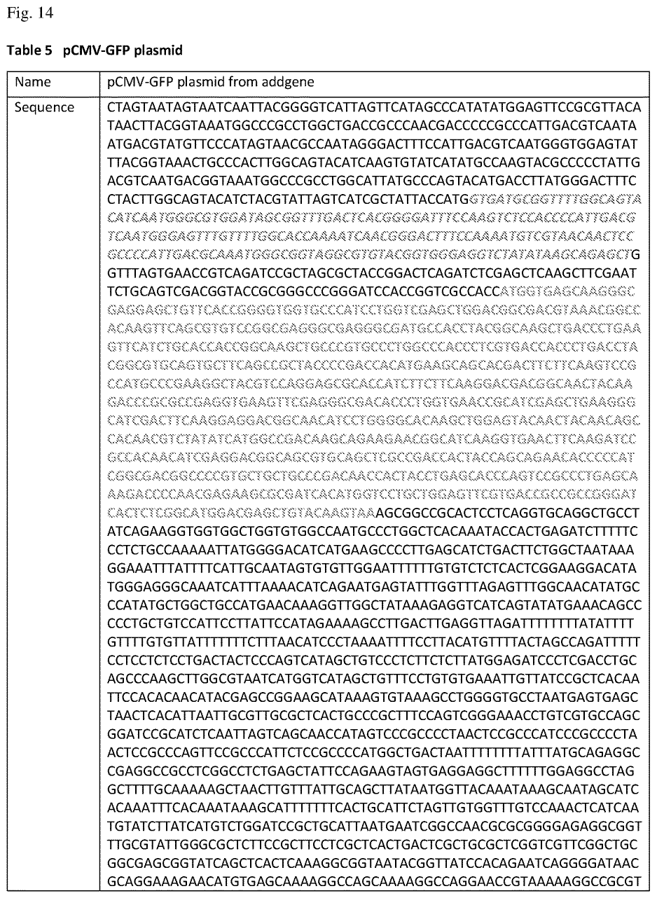

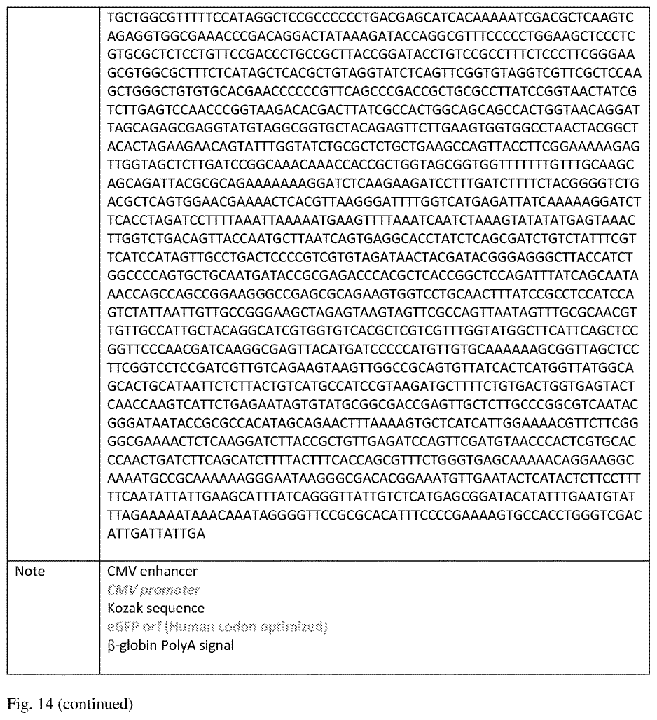

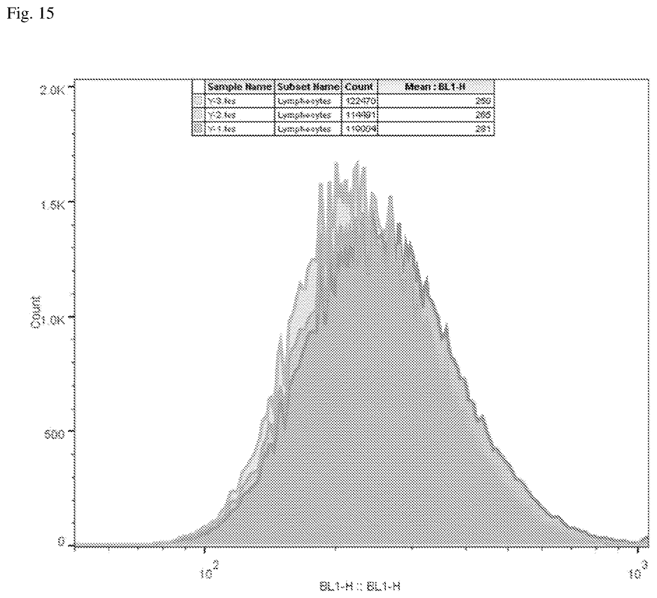

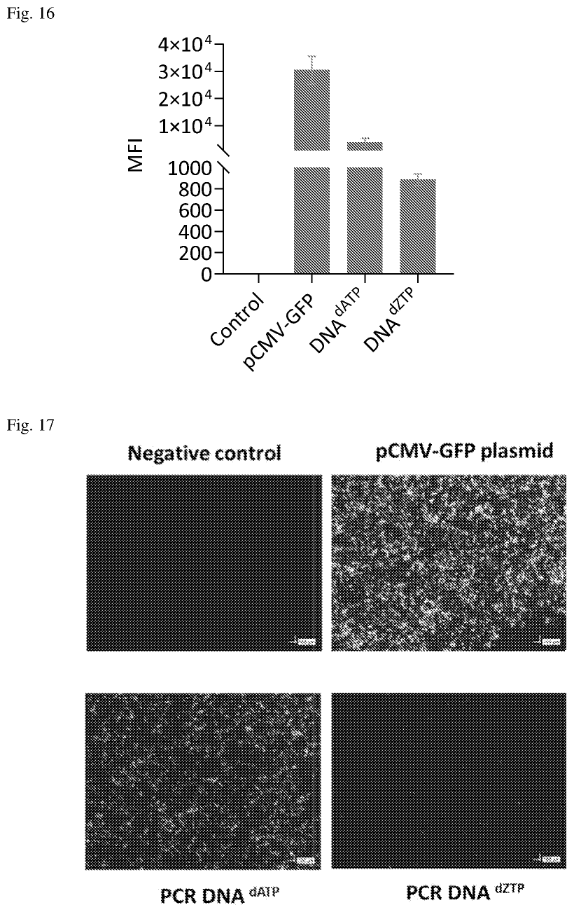

Building on these results, we next evaluated whether the element could work in cell system when A substituted at 100%. Three gene expression cassettes were designed, 1) T7p-eGFPEC-terminator for E. coli, 804 bp, AT at 55%, 2) TEF1p-eGFPSC-CYC1t for Saccharomyces cerevisiae (32), 1059 bp, AT at 62%, (FIG. 13), 3) CMVenhancer-CMVp-eGFPHS-terminator-polyA for Homo sapiens, 1640 bp, AT at 45% (FIG. 2b, Table 5). Primers located outside the cassettes were used to amplify the normal or Z-substituted DNA strands. Fluorescence was detected in E. coli cell free system and S. cerevisiae BY4742 transformation cell population (FIG. 15). Interestingly, when the HEK293 cell line was transfected with DNA-dZTP eGFPHS cassettes, there were 3.48% GFP positive cells with mean fluorescence intensity (MFI) lower 3.47 folds than DNA-dATP (FIG. 2g, 16, 17). These results indicate that transcription and translation system could work for Z-substituted gene expression regulators in both bacteria and eukaryotic organisms.

### Example 4: ZUGC mRNA could be Translated into Protein in Mammalian Cell

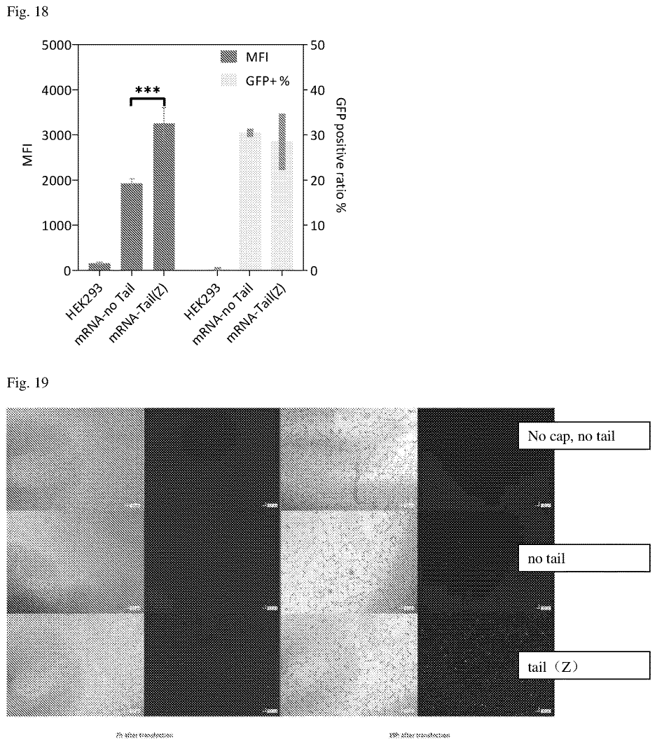

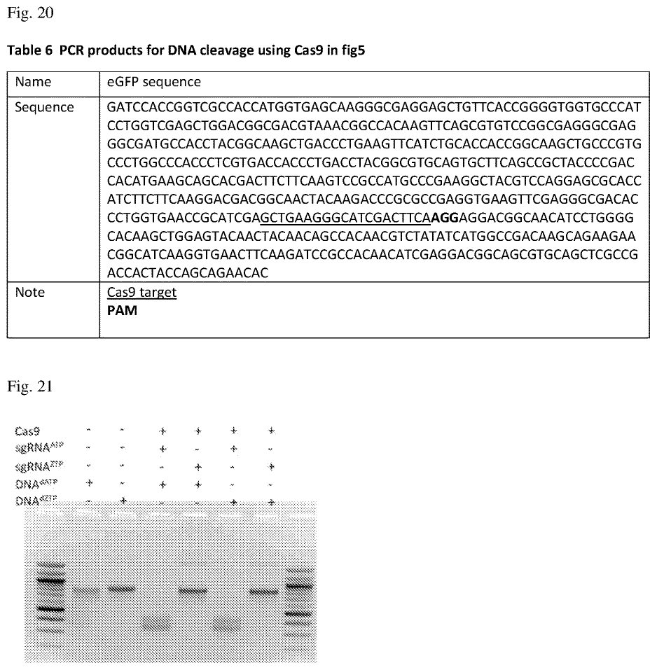

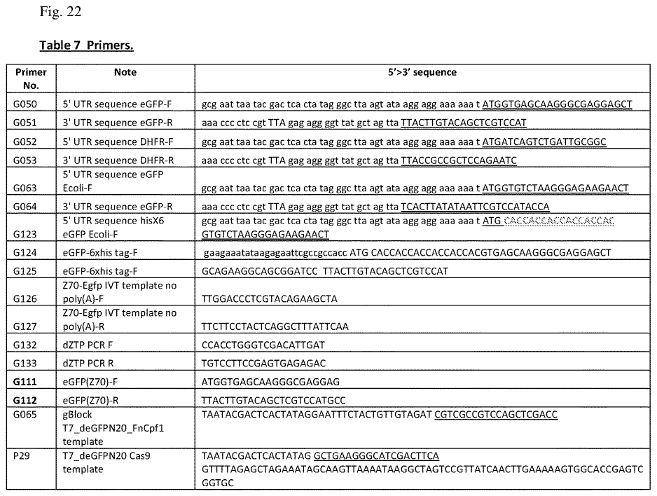

In Eukaryotic, Poly(A) tails have key roles in control of mRNA stability (33).Before investigation of ZTGC mRNA, we firstly explored whether ZTP could replace ATP and execute biological function in type of poly(Z) tail (FIG. 3a). Poly(A) polymerase from E. coli succeeded in tailing ZTP to normal mRNA strand with length of 875nt (FIG. 3b). 24 hours after transfection, poly(Z) tailing mRNA showed more 50% of GFP expression than no tailing mRNA (FIG. 18, 19).

Then we tried to make mRNA products using T7 RNA polymerase reaction volume. As expected, RNA templates were transcribed successfully in vitro when ZTP replaced by ATP completely (FIG. 3c). Modified nucleotides could induce decrease in yield of full-length transcription products (21). As a noncanonical nucleotide, ZTP also lead obviously decrease in yield of target mRNA transcript (unpublished data). To test whether Z-substituted mRNA was accepted to be translated in mammalian life, we tried delivery in to HEK293 cell line. HEK293 cells showed obvious GFP signal after transfected with mRNA-ZTP. Z-substituted mRNA resulted in 65.2% GFP positive, similarly to the normal mRNA, however the latter produced higher 9.13-fold in GFP protein expression level (FIG. 3d, e, f).

### Example 5: Z Base has No Effects on Fidelity PCR and In Vitro RNA Transcription

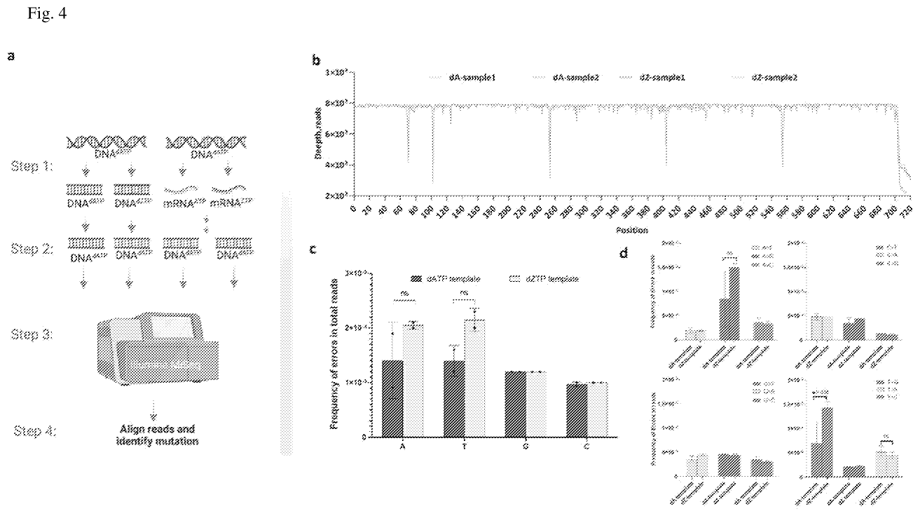

Fidelity of PCR and in vitro RNA transcription could be analyzed by NGS has been published in previous research (20; 21). We investigated whether Z base could lead more errors than A base (FIG. 4a). For PCR analysis, normal DNA products were prepared through PCR amplifications from DNA^{dATP} template respectively. For in vitro RNA transcription analysis, cDNA made by reverse-transcription were used as templates to prepare target PCR products. These products were analyzed by NGS. We obtained 1803 to 7697 reads for each position of the PCR products with 720 bp length (FIG. 4b). Error rates in reads were calculated and analyzed, results indicated that there were no significant differences between DNA^{dATP} and DNA^{dZTP} templates (FIG. 4c, 4d).

### Example 6: Z-Substituted sgRNA Enhances Cpf1 Cleave Activity

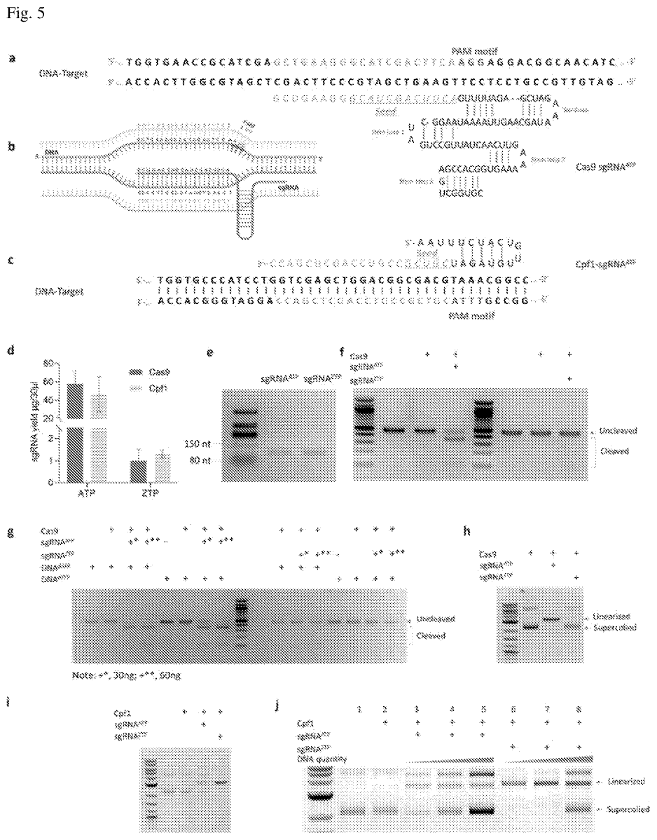

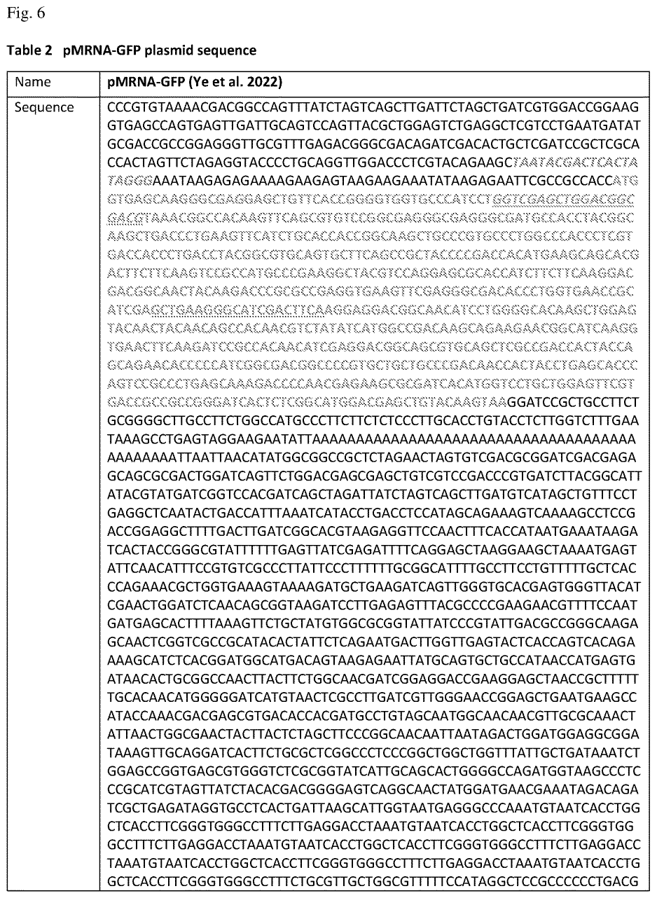

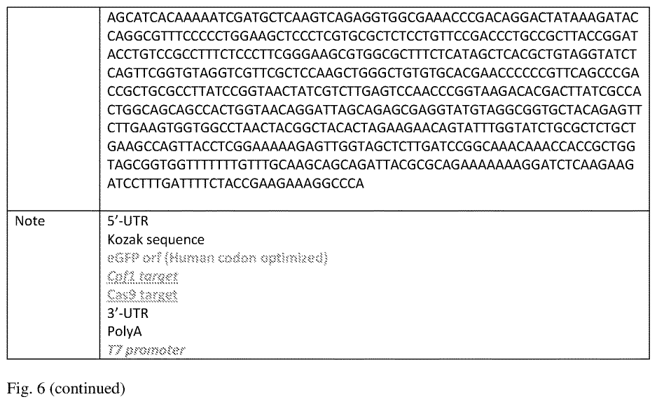

As a cyanobacterium, Synechococcus could be attacked by cyanophage S-2L. Native CRISPR system has been identified in some cyanobacterial genomes so far, except the Synechococcus (88; 89). Exploration of how Z base substitution effect RNA-guided endonuclease work for DNA substrate will be much helpful for understanding evolutionary and developing potential gene editing tools. Two single guide RNA (sgRNA) (47; 36), for SpCas9 and LbCpf1 respectively, targeting GFP sequence were designed (FIG. 5a, 5b, 5c and Table 6). RNA transcript templates were under control of T7 promoter to make sgRNA products using in vitro T7 RNA polymerase system. SpCas9-sgRNA was as long as 96nt with containing 32.3% A base, LbCpf1-sgRNA took 39nt length in which A occupied 6 positions. Target sgRNA was transcribed successfully for both SpCas9 and LbCpf1 duplex DNA oligonucleotides templates, however the yield decreased to 50-fold because of Z-substitution at 100% (FIG. 5d, 5e). We first examined the activity of Cas9 protein loaded two different sgRNAs to cleave a PCR amplicon of GFP region (Table 6) in vitro. We found that Cas9/sgRNAZIP showed no detectable activity to normal DNA substrate (FIG. 5f). We further studied effect of Z substitutions in target DNA on Cas9 RNA-guided DNA cleavage activity. Surprisingly Z-substitutions in the recognition region, including spacer and PAM of Cas9 did not affect nuclease activity (FIGS. 5g and 21). We investigated whether SpCas9 supplied with sgRNA-ATP can cleave target normal plasmid in vitro. We assayed its ability to cleave the same spacer-containing supercoiled plasmid pMRNA-eGFP. We found that SpCas9 only along with an in-vitro-transcribed mature sgRNA-ATP was able to efficiently cleave the plasmid substrate at the correct target site (FIG. 5h, Table 2). Compared with sgRNA for Cas9, which has four stem loop structure features that inter contact with Cas9 (48), the sgRNA for LbCpf1 only consists of a single stem loop in and is notably simpler (FIG. 5a, 5c). Next, we tested the DNA cleavage activity of Cpf1 programed with Z substituted sgRNA (FIG. 5i, 5j). We observe that sgRNA-ZTP was able to guide Cpf1-catalyzed DNA cleavage in a manner similar to that observed for the normal sgRNA-ATP. Interestingly, Cpf1 nuclease activity was improved 3-fold using Z-substituted sgRNA.

### Example 7: Comparison of Cleavage Activity Between ATP sgRNA and ZTP sgRNA Method of In Vitro Assay

27 μL volume containing 1.5 μmol LbCpf1, 2.5 μmol sgRNA (Table 8), 1x buffer r2.1, then incubated at 37 Celsius for 10 min. Then 300 ng DNA substrate in 3 μl was added to initial reaction. 6 μL sample was picked up at 5, 25, 50 and 90 min and reaction was stopped by adding 2 μL stop buffer. Then each sample was analyzed by gel electrophoresis. The cleavage percent was calculated using band intensity.

TABLE 8
sgRNA list

| | | GC % in | AT % | |
|---|---|---|---|---|
| | | guide | guide | |
| NO | TTTA, N20 | RNA | RNA | Guide RNA Sequence 5′ > 3′ |
| 1 | AT2 | 25 | 75 | TCAGATATTCTACTTTGTTT |
| 2 | AT6 | 20 | 80 | AACTATATTTTTTCACATAG |
| 3 | AT7 | 45 | 55 | CCCAGAGTCCTTCAAAGATA |
| 4 | AT10 | 30 | 70 | AGTTGTAGATATGATTTAGC |
| 5 | AT15 | 55 | 45 | AAGGCCAAACCTCGGCTTAC |
| 6 | AT23 | 15 | 85 | TTATATATGTGATACATTTT |

### Substrate

This plasmid was linearized by SspI and used as the substrate of cleavage activity assay.

pIDT-Dmd
TCGCGCGTTTCGGTGATGACGGTGAAAACCTCTGACACATGCAGCTCCCGGAGACGGTCACAGCTTGTCTGTAAG
CGGATGCCGGGAGCAGACAAGCCCGTCAGGGCGCGTCAGCGGGTGTTGGCGGGTGTCGGGGCTGGCTTAACTAT
GCGGCATCAGAGCAGATTGTACTGAGAGTGCACCAAATGCGGTGTGAAATACCGCACAGATGCGTAAGGAGAAA
ATACCGCATCAGGCGCCATTCGCCATTCAGGCTGCGCAACTGTTGGGAAGGGCGATCGGTGCGGGCCTCATCGCT
ATTACGCCAGCTGGCGAAAGGGGGATGTGCTGCAAGGCGATTAAGTTGGGTAACGCCAGGGTTTTCCCAGTCAC
GACGTTGTAAAACGACGGCCAGTGCAACGCGATGACGATGGATAGCGATTCATCGATGAGCTGACCCGATCGCC
GCCGCCGGAGGGTTGCGTTTGAGACGGGCGACAGATAATTAATAGAAGTCAATGTAGGGAAGGAAATATGGCAG
AAATTAAACATATATTCATAATTCAAAAGTGAAATATGGTAGTGAAAACATATGTCTGCCATATATATAATATATGC
ATATGTAATACATGTGTATATATGTATGCTGTGAGCTAAATCATATCTACAACTTAAAAAATGAACATAATGGAATA
AGGATATTTTAAATTATTATGTTAAAATGTTAAGTATACTTGGAGTTGGTGAAAAAAAATCAATTCATTTATACAGA
CAATCCAAGAAGGTATGACACATTACTGTTTTCATAGGAAAAATAGGCAAGTTGCAATCCTTTGAAATAGATTTGG
CTTTTGATATCATCAATATCTTTGAAGGACTCTGGGTAAAATATCTGTTTCCCATCACATTTTCCAATTAATTGCTAT
GTGAAAAAATATAGTTTAAAGGCCAAACCTCGGCTTACCTGAAATTTTCGAAGTTTATTCATATGTTCTTCTAGCTTT
TGGCAGCTTTCCACCAACTGGGAGGAAAGTTTCTTCCAGTGCCCCTCAATCTCTTCAAATTCTGACAGATATTTCTG
GCATATTTCTGAAGGTGCTTTCTTGGCCATCTCCTTCACAGTGTCACTCAGATAGTTGAAGCCATTTTGTTGCTCTTT
CAAAGAACTTTGCAGAGCCTCAAAATTAAATAGAAGTTCATTTACACTAACACGCATATTTGATGAGTTTCATTCAT
ATCAAGAAGAAGGTAAATAAAAGTTTATGTAATTTAGAGATCTAAACAAAGTAGAATATCTGATAAAGCAATATAT
ATGTATTCTTATGAATAATTTCTATTATATTACAGGGCATATTATATTTAGACAGAAATTTAGAAGTCATAATTTTAC
TGAATATCTATGCATTAATAACTATGTTTATTATATATGTGATACATTTTAACTTACCTACAGTGAAACATTATTAAG
ACATTTAGGCAATATATGATTAATATATTTTCAGATTTAAAAAGAATAAGTATACAACATGGGATTTTTAGAATCAA
CAAAAAAATTAGTCTTTATATGTCCTCACATCACAGAAGTTTCTCATCAGTTCTGGACCAGCGAGCTGTGCTGCGAC
TCGTGGCGTAATCATGGTCATAGCTGTTTCCTGTGTGAAATTGTTATCCGCTCACAATTCCACACAACATACGAGCC
GGAAGCATAAAGTGTAAAGCCTGGGGTGCCTAATGAGTGAGCTAACTCACATTAATTGCGTTGCGCTCACTGCCC
GCTTTCCAGTCGGGAAACCTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGATACCAGGCGTTTCCCCCTGGAAGC
TCCCTCGTGCGCTCTCCTGTTCCGACCCTGTCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAACCCGGTAAGACACGACTTATC
GCCACTGGCAGCAGCCACTGGTAACAGGATTAGCAGAGCGAGGTATGTAGGCGGTGCTACAGAGTTCTTGAAGT
GGTGGCCTAACTACGGCTACACTAGAAGAACAGTATTTGGTATCTGCGCTCTGCTGAAGCCAGTTACCTTCGGAAA
AAGAGTTGGTAGCTCTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCAGTGAGGCACCTATCTCAG
CGATCTGTCTATTTCGTTCATCCATAGTTGCCTGACTCCCCGTCGTGTAGATAACTACGATACGGGAGGGCTTACCA
TCTGGCCCCAGTGCTGCAATGATACCGCGAGACCCACGCTCACCGGCTCCAGATTTATCAGCAATAAACCAGCCAG
CCGGAAGGGCCGAGCGCAGAAGTGGTCCTGCAACTTTATCCGCCTCCATCCAGTCTATTAATTGTTGCCGGGAAGC
TAGAGTAAGTAGTTCGCCAGTTAATAGTTTGCGCAACGTTGTTGCCATTGCTACAGGCATCGTGGTGTCACGCTCG
TCGTTTGGTATGGCTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACTGGTGAGTACTCAACCAAGTCATT
CTGAGAATAGTGTATGCGGCGACCGAGTTGCTCTTGCCCGGCGTCAATACGGGATAATACCGCGCCACATAGCAG
AACTTTAAAAGTGCTCATCATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCAGCGTTTCTGGGTGAGCAAA
AACAGGAAGGCAAAATGCCGCAAAAAAGGGAATAAGGGCGACACGGAAATGTTGAATACTCATACTCTACCTTTT
TCAATATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCATTATTATCATGACATTAAC
CTATAAAAATAGGCGTATCACGAGGCCCTTTCGTC

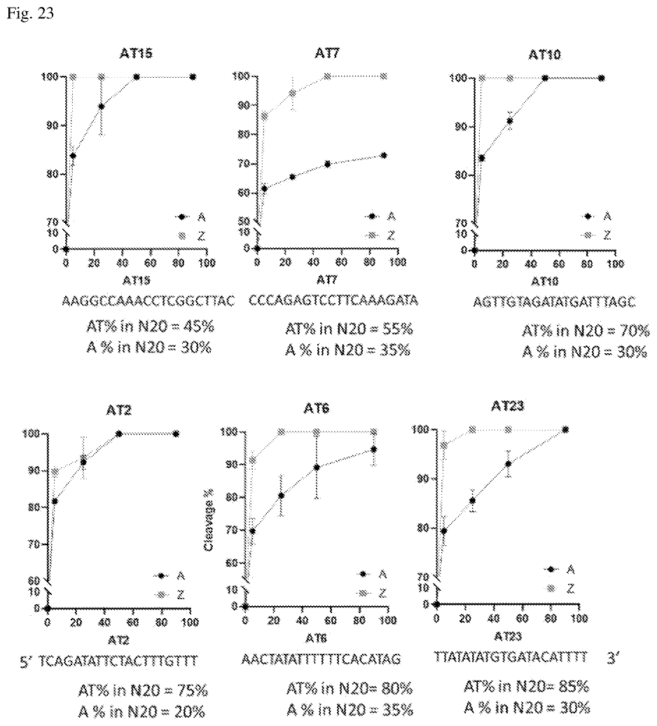

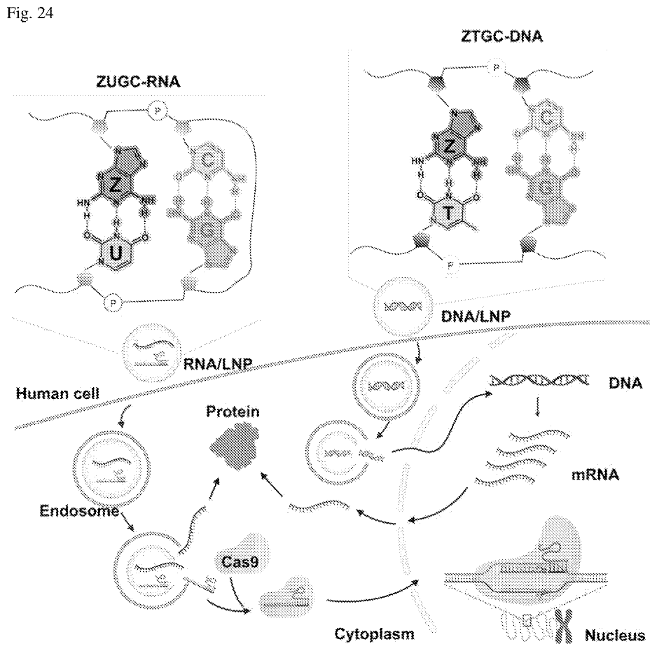

Cleavage activity for each guide RNA with ATP or ZTP is shown in FIG. 23. ZTP-incorporated sgRNA increased CRISPR-Cpf1 cleavage activity (FIG. 23).

Here we are the first time to demonstrate biological applications of ZTGC DNA or RNA in Prokaryote and Eukaryote by experiments. As proof of concept, we proved that ZTGC alphabet could be used to express proteins in living system including mammalian cells. RNA-guided endonucleases could utilize normal sgRNA RNA to cleave target Z-substituted DNA substrate, also could use ZUGC sgRNA to cleave plasmid DNA. Z base could replace A base and generate ZTGC or ZUGC alphabet. This type of alphabet could be used to express functional protein GFP in yeast and mammalian cell. Z base substituted sgRNA could be used by Cpf1 protein. Cpf1-sgRNAZIP complex could be targeted to cleave dsDNA. Could be used for CRISPR gene editing. mRNA could be added poly(Z) tail at 3′ end. Poly(z) could increase mRNA expression level in mammalian cell. Z RNA also could be used for RNAi and RNA editing. Animal in vivo test is undergoing.

### Example 8: Z-T Pairing could Reduce the Melting Temperature and Enhance Resistance to Type II REs

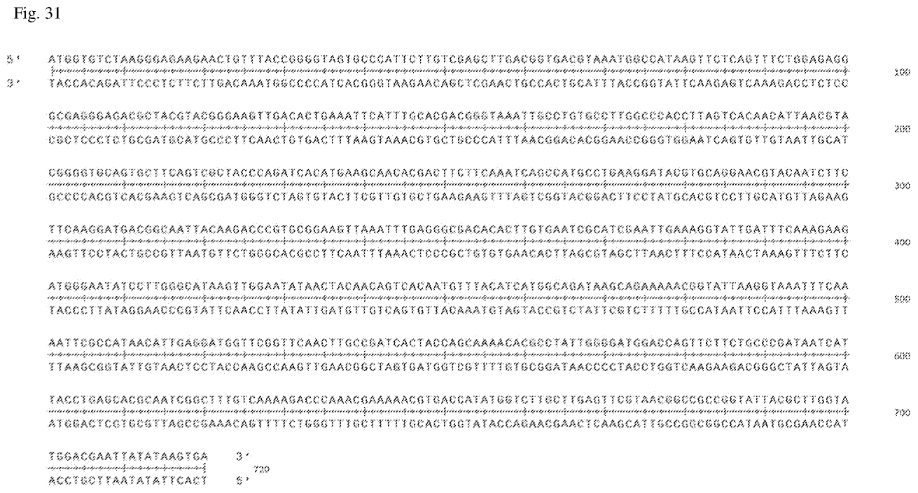

We first screened the ability of different DNA polymerases to prepare Z-DNA via in vitro reactions. Both A-family DNA polymerases, including Taq DNA polymerase from Thermus aquaticus YT-1, and B-family DNA polymerases, including Pfu polymerase from Pyrococcus furiosus, are able to incorporate modified nucleotides into DNA strands (19). We designed an artificial DNA template (DNA1) 720 base pairs (bp) long and with 54.4% AT content (FIG. 31). We then compared the polymerase chain reaction (PCR) amplification efficiency of Taq and Pfu on control and Z-substituted DNA1 templates. In this experiment, only Taq succeeded in amplification when dATP was completely replaced with dZTP for PCR. These results indicate that the Taq polymerase is more flexible than Pfu for dZTP incorporation.

We then evaluated the yield of PCR amplification using 11 templates (Table 9).

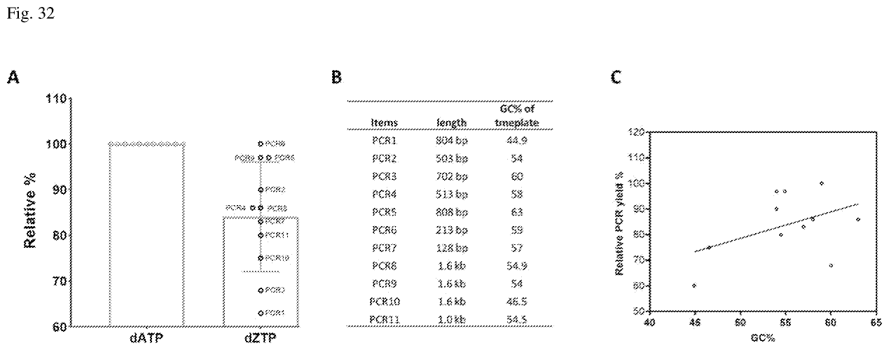

TABLE 9
PCR used in FIG. 32 and DHFR sequence.

| Items | Sequences 5′ > 3′ |
|---|---|
| PCR1; | ATGGTGTCTAAGGGAGAAGAACTGTTTACCGGGGTAGTGCCCATTCTTGTCGAGCTTGACGGTGACGTAA |
| G063/G064 | ATGGCCATAAGTTCTCAGTTTCTGGAGAGGGCGAGGGAGACGCTACGTACGGGAAGTTGACACTGAAAT |
| primer | TCATTTGCACGACGGGTAAATTGCCTGTGCCTTGGCCCACCTTAGTCACAACATTAACGTACGGGGTGCAG |
| pairs; | TGCTTCAGTCGCTACCCAGATCACATGAAGCAACACGACTTCTTCAAATCAGCCATGCCTGAAGGATACGT |
| Length, 804 | GCAGGAACGTACAATCTTCTTCAAGGATGACGGCAATTACAAGACCCGTGCGGAAGTTAAATTTGAGGGC |
| bp | GACACACTTGTGAATCGCATCGAATTGAAAGGTATTGATTTCAAAGAAGATGGGAATATCCTTGGGCATA |
| | AGTTGGAATATAACTACAACAGTCACAATGTTTACATCATGGCAGATAAGCAGAAAAACGGTATTAAGGT |
| | AAATTTCAAAATTCGCCATAACATTGAGGATGGTTCGGTTCAACTTGCCGATCACTACCAGCAAAACACGC |
| | CTATTGGGGATGGACCAGTTCTTCTGCCCGATAATCATTACCTGAGCACGCAATCGGCTTTGTCAAAAGAC |
| | CCAAACGAAAAACGTGACCATATGGTCTTGCTTGAGTTCGTAACGGCCGCCGGTATTACGCTTGGTATGG |
| | ACGAATTATATAAGTGA |
| PCR2 | GTAAAACGACGGCCAGTGCAACGCGATGACGATGGATAGCGATTCATCGATGAGCTGACCCGATCGCCG |
| G097/G098 | CCGCCGGAGGGTTGCGTTTGAGACGGGCGACAGATAGCGGCCGCGAATTCCTAGAGCTCGCTGATCAGC |
| Length, 503 | CTCGACTGTGCCTTCTAGTTGCCAGCCATCTGTTGTTTGCCCCTCCCCCGTGCCTTCCTTGACCCTGGAAGG |
| bp | TGCCACTCCCACTGTCCTTTCCTAATAAAATGAGGAAATTGCATCGCATTGTCTGAGTAGGTGTCATTCTAT |
| | TCTGGGGGGTGGGGTGGGGCAGGACAGCAAGGGGGAGGATTGGGAAGAGAATAGCAGGCATGCTGGG |
| | GATGTCTCTCACTCGGAAGGACATATGGGAGGGCAAATCATTTAAAACATCAGAATGAGTATTTGGTTTA |
| | GAGTTTGGCAACATATGCCATCAGTTCTGGACCAGCGAGCTGTGCTGCGACTCGTGGCGTAATCATGGTC |
| | ATAGCTGTTTCCTG |
| PCR3 | GTAAAACGACGGCCAGTGCAACGCGATGACGATGGATAGCGATTCATCGATGAGCTGACCCGATCGCCG |
| G097/G098 | CCGCCGGAGGGTTGCGTTTGAGACGGGCGACAGATCTACGTATTAGTCATCGCTATTACCATGGTGGCCT |
| Length, 702 | CCGCGCCGGGTTTTGGCGCCTCCCGCGGGCGCCCCCCTCCTCACGGCGAGCGCTGCCACGTCAGACGAAG |
| bp | GGCGCAGCGAGCGTCCTGATCCTTCCGCCCGGACGCTCAGGACAGCGGCCCGCTGCTCATAAGACTCGGC |
| | CTTAGAACCCCAGTATCAGCAGAAGGACATTTTAGGACGGGACTTGGGTGACTCTAGGGCACTGGTTTTC |
| | TTTCCAGAGAGCGGAACAGGCGAGGAAAAGTAGTCCCTTCTCGGCGATTCTGCGGAGGGATCTCCGTGG |
| | GGCGGTGAACGCCGATGATTATATAAGGACGCGCCGGGTGTGGCACAGCTAGTTCCGTCGCAGCCGGGA |
| | TTTGGGTCGCGGTTCTTGTTTGTGGATCGCTGTGATCGTCACTTGGGGTTTAGTGAACCGTCAGATCCGCT |
| | AGCGCTACCGGACTCAGATCTCGAGCTCAAGCTTCGAATTCTGCAGTCGACGGTACCGCGGGCCCGGGAT |
| | CCACCGGTCATCAGTTCTGGACCAGCGAGCTGTGCTGCGACTCGTGGCGTAATCATGGTCATAGCTGTTTC |
| | CTG |
| PCR4 | GTAAAACGACGGCCAGTGCAACGCGATGACGATGGATAGCGATTCATCGATGAGCTGACCCGATCGCCG |
| G097/G098 | CCGCCGGAGGGTTGCGTTTGAGACGGGCGACAGATCTACGTATTAGTCATCGCTATTACCATGGGGCAGA |
| Length, 513 | GCGCACATCGCCCACAGTCCCCGAGAAGTTGGGGGGAGGGGTCGGCAATTGATCCGGTGCCTAGAGAAG |
| bp | GTGGCGCGGGGTAAACTGGGAAAGTGATGTCGTGTACTGGCTCCGCCTTTTTCCCGAGGGTGGGGGAGA |
| | ACCGTATATAAGTGCAGTAGTCGCCGTGAACGTTCTTTTTCGCAACGGGTTTGCCGCCAGAACACAGGGTT |
| | TAGTGAACCGTCAGATCCGCTAGCGCTACCGGACTCAGATCTCGAGCTCAAGCTTCGAATTCTGCAGTCGA |
| | CGGTACCGCGGGCCCGGGATCCACCGGTCATCAGTTCTGGACCAGCGAGCTGTGCTGCGACTCGTGGCGT |
| | AATCATGGTCATAGCTGTTTCCTG |
| PCR5 | GTAAAACGACGGCCAGTGCAACGCGATGACGATGGATAGCGATTCATCGATGAGCTGACCCGATCGCCG |
| G097/G098 | CCGCCGGAGGGTTGCGTTTGAGACGGGCGACAGATCCATCCCCTTCTGTGAATGTTAGACCCATGGGAGC |
| Length, 808 | AGCTGGTCAGAGGGGACCCCGGCCTGGGGCCCCTAACCCTATGTAGCCTCAGTCTTCCCATCAGGCTCTCA |
| bp | GCTCAGCCTGAGTGTTGAGGCCCCAGTGGCTGCTCTGGGGGCCTCCTGAGTTTCTCATCTGTGCCCCTCCC |
| | TCCCTGGCCCAGGTGAAGGTGTGGTTCCAGAACCGGAGGACAAAGTACAAACGGCAGAAGCTGGAGGA |
| | GGAAGGGCCTGAGTCCGAGCAGAAGAAGAAGGGCTCCCATCACATCAACCGGTGGCGCATTGCCACGAA |
| | GCAGGCCAATGGGGAGGACATCGATGTCACCTCCAATGACTAGGGTGGGCAACCACAAACCCACGAGGG |
| | CAGAGTGCTGCTTGCTGCTGGCCAGGCCCCTGCGTGGGCCCAAGCTGGACTCTGGCCACTCCCTGGCCAG |
| | GCTTTGGGGAGGCCTGGAGTCATGGCCCCACAGGGCTTGAAGCCCGGGGCCGCCATTGACAGAGGGACA |
| | AGCAATGGGCTGGCTGAGGCCTGGGACCACTTGGCCTTCTCCTCGGAGAGCCTGCCTGCCTGGGCGGGC |
| | CCGCCCGCCACCGCAGCCTCCCAGCTGCTCTCCGTGTCTCCAATCTCCATCAGTTCTGGACCAGCGAGCTGT |
| | GCTGCGACTCGTGGCGTAATCATGGTCATAGCTGTTTCCTG |
| PCR6 | GCCTCCTGAGTTTCTCATCTGTGCCCCTCCCTCCCTGGCCCAGGTGAAGGTGTGGTTCCAGAACCGGAGGA |
| G083/G084 | CAAAGTACAAACGGCAGAAGCTGGAGGAGGAAGGGCCTGAGTCCGAGCAGAAGAAGAAGGGCTCCCAT |
| Length, 213 | CACATCAACCGGTGGCGCATTGCCACGAAGCAGGCCAATGGGGAGGACATCGATGTCACCTCCAATGACT |
| bp | AGGG |
| PCR7 | GGAGGACAAAGTACAAACGGCAGAAGCTGGAGGAGGAAGGGCCTGAGTCCGAGCAGAAGAAGAAGGG |
| G103/G104 | CTCCCATCACATCAACCGGTGGCGCATTGCCACGAAGCAGGCCAATGGGGAGGACATCGAT |
| Length, 128 | |
| bp | |
| PCR8 | CCACCTGGGTCGACATTGATTATTGACTAGTAATAGTAATCAATTACGGGGTCATTAGTTCATAGCCCATA |
| G132/G133 | TATGGAGTTCCGCGTTACATAACTTACGGTAAATGGCCCGCCTGGCTGACCGCCCAACGACCCCCGCCCAT |
| Length, 1.6 | TGACGTCAATAATGACGTATGTTCCCATAGTAACGCCAATAGGGACTTTCCATTGACGTCAATGGGTGGA |
| kb | GTATTTACGGTAAACTGCCCACTTGGCAGTACATCAAGTGTATCATATGCCAAGTACGCCCCCTATTGACG |
| | TCAATGACGGTAAATGGCCCGCCTGGCATTATGCCCAGTACATGACCTTATGGGACTTTCCTACTTGGCAG |
| | TACATCTACGTATTAGTCATCGCTATTACCATGGTGATGCGGTTTTGGCAGTACATCAATGGGCGTGGATA |
| | GCGGTTTGACTCACGGGGATTTCCAAGTCTCCACCCCATTGACGTCAATGGGAGTTTGTTTTGGCACCAAA |
| | ATCAACGGGACTTTCCAAAATGTCGTAACAACTCCGCCCCATTGACGCAAATGGGCGGTAGGCGTGTACG |
| | GTGGGAGGTCTATATAAGCAGAGCTGGTTTAGTGAACCGTCAGATCCGCTAGCGCTACCGGACTCAGATC |
| | TCGAGCTCAAGCTTCGAATTCTGCAGTCGACGGTACCGCGGGCCCGGGATCCACCGGTCGCCACCATGGT |
| | GAGCAAGGGCGAGGAGCTGTTCACCGGGGTGGTGCCCATCCTGGTCGAGCTGGACGGCGACGTAAACG |
| | GCCACAAGTTCAGCGTGTCCGGCGAGGGCGAGGGCGATGCCACCTACGGCAAGCTGACCCTGAAGTTCA |
| | TCTGCACCACCGGCAAGCTGCCCGTGCCCTGGCCCACCCTCGTGACCACCCTGACCTACGGCGTGCAGTGC |
| | TTCAGCCGCTACCCCGACCACATGAAGCAGCACGACTTCTTCAAGTCCGCCATGCCCGAAGGCTACGTCCA |
| | GGAGCGCACCATCTTCTTCAAGGACGACGGCAACTACAAGACCCGCGCCGAGGTGAAGTTCGAGGGCGA |
| | CACCCTGGTGAACCGCATCGAGCTGAAGGGCATCGACTTCAAGGAGGACGGCAACATCCTGGGGCACAA |
| | GCTGGAGTACAACTACAACAGCCACAACGTCTATATCATGGCCGACAAGCAGAAGAACGGCATCAAGGT |
| | GAACTTCAAGATCCGCCACAACATCGAGGACGGCAGCGTGCAGCTCGCCGACCACTACCAGCAGAACACC |
| | CCCATCGGCGACGGCCCCGTGCTGCTGCCCGACAACCACTACCTGAGCACCCAGTCCGCCCTGAGCAAAG |
| | ACCCCAACGAGAAGCGCGATCACATGGTCCTGCTGGAGTTCGTGACCGCCGCCGGGATCACTCTCGGCAT |
| | GGACGAGCTGTACAAGTAAAGCGGCCGCACTCCTCAGGTGCAGGCTGCCTATCAGAAGGTGGTGGCTGG |
| | TGTGGCCAATGCCCTGGCTCACAAATACCACTGAGATCTTTTTCCCTCTGCCAAAAATTATGGGGACATCAT |
| | GAAGCCCCTTGAGCATCTGACTTCTGGCTAATAAAGGAAATTTATTTTCATTGCAATAGTGTGTTGGAATTT |
| | TTTGTGTCTCTCACTCGGAAGGACA |
| PCR9 | CCACCTGGGTCGACATTGATTATTGACTAGTTGTTACATAACTTATGGTAAATGGCCTGCCTGGCTGACTG |
| G132/G133 | CCCAATGACCCCTGCCCATTGATGTCAATAATGATGTATGTTCCCATAGTAATGCCAATAGGGACTTTCCAT |
| Length, 1.6 | TGATGTCAATGGGTGGAGTATTTATGGTAAACTGCCCACTTGGCAGTACATCAAGTGTATCATATGCCAAG |
| kb | TATGCCCCCTATTGATGTCAATGATGGTAAATGGCCTGCCTGGCATTATGCCCAGTACATGACCTTATGGG |
| | ACTTTCCTACTTGGCAGTACATCTATGTATTAGTCATTGCTATTACCATGACTAGTGGAGAAGAGCATGCTT |
| | GAGGGCTGAGTGCCCCTCAGTGGGCAGAGAGCACATGGCCCACAGTCCCTGAGAAGTTGGGGGGAGGG |
| | GTGGGCAATTGAACTGGTGCCTAGAGAAGGTGGGGCTTGGGTAAACTGGGAAAGTGATGTGGTGTACTG |
| | GCTCCACCTTTTTCCCCAGGGTGGGGGAGAACCATATATAAGTGCAGTAGTCTCTGTGAACATTCAAGCTT |
| | CTGCCTTCTCCCTCCTGTGAGTTTGGTAAGTCACTGACTGTCTATGCCTGGGAAAGGGTGGGCAGGAGGT |
| | GGGGCAGTGCAGGAAAAGTGGCACTGTGAACCCTGCAGCCCTAGACAATTGTACTAACCTTCTTCTCTTTC |
| | CTCTCCTGACAGACCGGTCGCCACCATGGTGAGCAAGGGCGAGGAGCTGTTCACCGGGGTGGTGCCCAT |
| | CCTGGTCGAGCTGGACGGCGACGTAAACGGCCACAAGTTCAGCGTGTCCGGCGAGGGCGAGGGCGATG |
| | CCACCTACGGCAAGCTGACCCTGAAGTTCATCTGCACCACCGGCAAGCTGCCCGTGCCCTGGCCCACCCTC |
| | GTGACCACCCTGACCTACGGCGTGCAGTGCTTCAGCCGCTACCCCGACCACATGAAGCAGCACGACTTCTT |
| | CAAGTCCGCCATGCCCGAAGGCTACGTCCAGGAGCGCACCATCTTCTTCAAGGACGACGGCAACTACAAG |
| | ACCCGCGCCGAGGTGAAGTTCGAGGGCGACACCCTGGTGAACCGCATCGAGCTGAAGGGCATCGACTTC |
| | AAGGAGGACGGCAACATCCTGGGGCACAAGCTGGAGTACAACTACAACAGCCACAACGTCTATATCATG |
| | GCCGACAAGCAGAAGAACGGCATCAAGGTGAACTTCAAGATCCGCCACAACATCGAGGACGGCAGCGTG |
| | CAGCTCGCCGACCACTACCAGCAGAACACCCCCATCGGCGACGGCCCCGTGCTGCTGCCCGACAACCACT |
| | ACCTGAGCACCCAGTCCGCCCTGAGCAAAGACCCCAACGAGAAGCGCGATCACATGGTCCTGCTGGAGTT |
| | CGTGACCGCCGCCGGGATCACTCTCGGCATGGACGAGCTGTACAAGTAAAGCGGCCGCACTCCTCAGGTG |
| | CAGGCTGCCTATCAGAAGGTGGTGGCTGGTGTGGCCAATGCCCTGGCTCACAAATACCACTGAGATCTTT |
| | TTCCCTCTGCCAAAAATTATGGGGACATCATGAAGCCCCTTGAGCATCTGACTTCTGGCTAATAAAGGAAA |
| | TTTATTTTCATTGCAATAGTGTGTTGGAATTTTTTGTGTCTCTCACTCGGAAGGACA |
| PCR10 | CCACCTGGGTCGACATTGATTATTGACTAGTTGTTACATAACTTATGGTAAATGGCCTGCCTGGCTGACTG |
| G132/G133 | CCCAATGACCCCTGCCCATTGATGTCAATAATGATGTATGTTCCCATAGTAATGCCAATAGGGACTTTCCAT |
| Length, 1.6 | TGATGTCAATGGGTGGAGTATTTATGGTAAACTGCCCACTTGGCAGTACATCAAGTGTATCATATGCCAAG |
| kb | TATGCCCCCTATTGATGTCAATGATGGTAAATGGCCTGCCTGGCATTATGCCCAGTACATGACCTTATGGG |
| | ACTTTCCTACTTGGCAGTACATCTATGTATTAGTCATTGCTATTACCATGACTAGTGGAGAAGAGCATGCTT |
| | GAGGGCTGAGTGCCCCTCAGTGGGCAGAGAGCACATGGCCCACAGTCCCTGAGAAGTTGGGGGGAGGG |
| | GTGGGCAATTGAACTGGTGCCTAGAGAAGGTGGGGCTTGGGTAAACTGGGAAAGTGATGTGGTGTACTG |
| | GCTCCACCTTTTTCCCCAGGGTGGGGGAGAACCATATATAAGTGCAGTAGTCTCTGTGAACATTCAAGCTT |
| | CTGCCTTCTCCCTCCTGTGAGTTTGGTAAGTCACTGACTGTCTATGCCTGGGAAAGGGTGGGCAGGAGGT |
| | GGGGCAGTGCAGGAAAAGTGGCACTGTGAACCCTGCAGCCCTAGACAATTGTACTAACCTTCTTCTCTTTC |
| | CTCTCCTGACAGACCGGTCGCCACCATGGTTTCTAAGGGAGAAGAACTCTTTACTGGTGTTGTCCCAATTC |
| | TGGTTGAGCTGGATGGTGATGTGAATGGCCACAAATTCTCTGTGTCTGGTGAAGGTGAAGGAGATGCAA |
| | CTTATGGAAAGCTGACTCTGAAGTTCATTTGTACAACAGGAAAGCTGCCAGTGCCTTGGCCAACTCTGGTG |
| | ACCACCCTGACTTATGGTGTTCAATGTTTCAGCAGGTACCCTGACCACATGAAGCAGCATGACTTCTTTAA |
| | ATCTGCAATGCCAGAAGGTTATGTTCAGGAGAGGACAATCTTCTTTAAGGATGATGGAAATTATAAGACA |
| | AGGGCAGAAGTGAAGTTTGAAGGTGATACACTGGTTAACAGAATTGAGCTGAAAGGCATTGATTTTAAG |
| | GAAGATGGAAACATTCTGGGTCACAAGCTGGAGTACAACTATAATTCTCACAATGTTTACATTATGGCAGA |
| | TAAGCAGAAGAATGGAATTAAGGTTAATTTCAAGATTAGACACAACATTGAGGATGGATCTGTCCAACTG |
| | GCAGACCATTACCAGCAGAACACCCCTATTGGTGATGGCCCAGTTCTCCTCCCAGATAATCACTATCTCAG |
| | CACTCAATCTGCTCTGTCCAAAGACCCTAATGAGAAAAGAGACCACATGGTCCTCCTGGAGTTTGTGACAG |
| | CAGCAGGAATTACTCTGGGAATGGATGAGCTGTACAAGTAAAGCGGCCGCACTCCTCAGGTGCAGGCTG |
| | CCTATCAGAAGGTGGTGGCTGGTGTGGCCAATGCCCTGGCTCACAAATACCACTGAGATCTTTTTCCCTCT |
| | GCCAAAAATTATGGGGACATCATGAAGCCCCTTGAGCATCTGACTTCTGGCTAATAAAGGAAATTTATTTT |
| | CATTGCAATAGTGTGTTGGAATTTTTTGTGTCTCTCACTCGGAAGGACA |
| PCR11 | CTAGTAATAGTAATCAATTACGGGGTCATTAGTTCATAGCCCATATATGGAGTTCCGCGTTACATAACTTAC |
| G132 | GGTAAATGGCCCGCCTGGCTGACCGCCCAACGACCCCCGCCCATTGACGTCAATAATGACGTATGTTCCCA |
| EGFP-R | TAGTAACGCCAATAGGGACTTTCCATTGACGTCAATGGGTGGAGTATTTACGGTAAACTGCCCACTTGGCA |
| Length, 1.0 | GTACATCAAGTGTATCATATGCCAAGTACGCCCCCTATTGACGTCAATGACGGTAAATGGCCCGCCTGGCA |
| kb | TTATGCCCAGTACATGACCTTATGGGACTTTCCTACTTGGCAGTACATCTACGTATTAGTCATCGCTATTAC |
| | CATGGTGATGCGGTTTTGGCAGTACATCAATGGGCGTGGATAGCGGTTTGACTCACGGGGATTTCCAAGT |
| | CTCCACCCCATTGACGTCAATGGGAGTTTGTTTTGGCACCAAAATCAACGGGACTTTCCAAAATGTCGTAA |
| | CAACTCCGCCCCATTGACGCAAATGGGCGGTAGGCGTGTACGGTGGGAGGTCTATATAAGCAGAGCTGG |
| | TTTAGTGAACCGTCAGATCCGCTAGCGCTACCGGACTCAGATCTCGAGCTCAAGCTTCGAATTCTGCAGTC |
| | GACGGTACCGCGGGCCCGGGATCCACCGGTCGCCACCATGGTGAGCAAGGGCGAGGAGCTGTTCACCGG |
| | GGTGGTGCCCATCCTGGTCGAGCTGGACGGCGACGTAAACGGCCACAAGTTCAGCGTGTCCGGCGAGGG |
| | CGAGGGCGATGCCACCTACGGCAAGCTGACCCTGAAGTTCATCTGCACCACCGGCAAGCTGCCCGTGCCC |
| | TGGCCCACCCTCGTGACCACCCTGACCTACGGCGTGCAGTGCTTCAGCCGCTACCCCGACCACATGAAGCA |
| | GCACGACTTCTTCAAGTCCGCCATGCCCGAAGGCTACGTCCAGGAGCGCACCATCTTCTTCAAGGACGAC |
| | GGCAACTACAAGACCCGCGCCGAGGTGAAGTTCGAGGGCGACACCCTGGTGAACCGCATCGAGCTGAAG |
| | GGCATCGACTTCAAGGAGGACGGCAACATCCTGGGGCACAAGCTGGAGTACAACTACAACAGCCACAAC |
| | GTCTATATCATGGCCGACAAGCAGAAGAACGGCATCAAGGTGAACTTCAAGATCCGCCACAACATCGAGG |
| | ACGGCAGCGTGCAGCTCGCCGACCACTACCAGCAGAACACCCCCATCGGCGACGGCCCCGTGCTGCTGCC |
| | CGACAACCACTACCTGAGCACCCAGTCCGCCCTGAGCAAAGACCCCAACGAGAAGCGCGATCACATGGTC |
| | CTGCTGGAGTTCGTGACCGCCGCCGGGATCACTCTCGGCATGGACGAGCTGTACAAGTAAAGCGGCCGC |
| | ACTCCTCAGGTGCAGGCTGCCTATCAGAAGGTGGTGGCTGGTGTGGCCAATGCCCTGGCTCACAAATACC |
| | ACTGAGATCTTTTTCCCTCTGCCAAAAATTATGGGGACATCATGAAGCCCCTTGAGCATCTGACTTCTGGCT |
| | AATAAAGGAAATTTATTTTCATTGCAATAGTGTGTTGGAATTTTTTGTGTCTCTCACTCGGAAGGACATATG |
| | GGAGGGCAAATCATTTAAAACATCAGAATGAGTATTTGGTTTAGAGTTTGGCAACATATGCCCATATGCTG |
| | GCTGCCATGAACAAAGGTTGGCTATAAAGAGGTCATCAGTATATGAAACAGCCCCCTGCTGTCCATTCCTT |
| | ATTCCATAGAAAAGCCTTGACTTGAGGTTAGATTTTTTTTATATTTTGTTTTGTGTTATTTTTTTCTTTAACAT |
| | CCCTAAAATTTTCCTTACATGTTTTACTAGCCAGATTTTTCCTCCTCTCCTGACTACTCCCAGTCATAGCTGTC |
| | CCTCTTCTCTTATGGAGATCCCTCGACCTGCAGCCCAAGCTTGGCGTAATCATGGTCATAGCTGTTTCCTGT |
| | GTGAAATTGTTATCCGCTCACAATTCCACACAACATACGAGCCGGAAGCATAAAGTGTAAAGCCTGGGGT |
| | GCCTAATGAGTGAGCTAACTCACATTAATTGCGTTGCGCTCACTGCCCGCTTTCCAGTCGGGAAACCTGTC |
| | GTGCCAGCGGATCCGCATCTCAATTAGTCAGCAACCATAGTCCCGCCCCTAACTCCGCCCATCCCGCCCCT |
| | AACTCCGCCCAGTTCCGCCCATTCTCCGCCCCATGGCTGACTAATTTTTTTTATTTATGCAGAGGCCGAGGC |
| | CGCCTCGGCCTCTGAGCTATTCCAGAAGTAGTGAGGAGGCTTTTTTGGAGGCCTAGGCTTTTGCAAAAAG |
| | CTAACTTGTTTATTGCAGCTTATAATGGTTACAAATAAAGCAATAGCATCACAAATTTCACAAATAAAGCAT |
| | TTTTTTCACTGCATTCTAGTTGTGGTTTGTCCAAACTCATCAATGTATCTTATCATGTCTGGATCCGCTGCAT |
| | TAATGAATCGGCCAACGCGCGGGGAGAGGCGGTTTGCGTATTGGGCGCTCTTCCGCTTCCTCGCTCACTG |
| | ACTCGCTGCGCTCGGTCGTTCGGCTGCGGCGAGCGGTATCAGCTCACTCAAAGGCGGTAATACGGTTATC |
| | CACAGAATCAGGGGATAACGCAGGAAAGAACATGTGAGCAAAAGGCCAGCAAAAGGCCAGGAACCGTA |
| | AAAAGGCCGCGTTGCTGGCGTTTTTCCATAGGCTCCGCCCCCCTGACGAGCATCACAAAAATCGACGCTCA |
| | AGTCAGAGGTGGCGAAACCCGACAGGACTATAAAGATACCAGGCGTTTCCCCCTGGAAGCTCCCTCGTGC |
| | GCTCTCCTGTTCCGACCCTGCCGCTTACCGGATACCTGTCCGCCTTTCTCCCTTCGGGAAGCGTGGCGCTTT |
| | CTCATAGCTCACGCTGTAGGTATCTCAGTTCGGTGTAGGTCGTTCGCTCCAAGCTGGGCTGTGTGCACGAA |
| | CCCCCCGTTCAGCCCGACCGCTGCGCCTTATCCGGTAACTATCGTCTTGAGTCCAACCCGGTAAGACACGA |
| | CTTATCGCCACTGGCAGCAGCCACTGGTAACAGGATTAGCAGAGCGAGGTATGTAGGCGGTGCTACAGA |
| | GTTCTTGAAGTGGTGGCCTAACTACGGCTACACTAGAAGAACAGTATTTGGTATCTGCGCTCTGCTGAAGC |
| | CAGTTACCTTCGGAAAAAGAGTTGGTAGCTCTTGATCCGGCAAACAAACCACCGCTGGTAGCGGTGGTTT |
| | TTTTGTTTGCAAGCAGCAGATTACGCGCAGAAAAAAAGGATCTCAAGAAGATCCTTTGATCTTTTCTACGG |
| | GGTCTGACGCTCAGTGGAACGAAAACTCACGTTAAGGGATTTTGGTCATGAGATTATCAAAAAGGATCTT |
| | CACCTAGATCCTTTTAAATTAAAAATGAAGTTTTAAATCAATCTAAAGTATATATGAGTAAACTTGGTCTGA |
| | CAGTTACCAATGCTTAATCAGTGAGGCACCTATCTCAGCGATCTGTCTATTTCGTTCATCCATAGTTGCCTG |
| | ACTCCCCGTCGTGTAGATAACTACGATACGGGAGGGCTTACCATCTGGCCCCAGTGCTGCAATGATACCG |
| | CGAGACCCACGCTCACCGGCTCCAGATTTATCAGCAATAAACCAGCCAGCCGGAAGGGCCGAGCGCAGA |
| | AGTGGTCCTGCAACTTTATCCGCCTCCATCCAGTCTATTAATTGTTGCCGGGAAGCTAGAGTAAGTAGTTC |
| | GCCAGTTAATAGTTTGCGCAACGTTGTTGCCATTGCTACAGGCATCGTGGTGTCACGCTCGTCGTTTGGTA |
| | TGGCTTCATTCAGCTCCGGTTCCCAACGATCAAGGCGAGTTACATGATCCCCCATGTTGTGCAAAAAAGCG |
| | GTTAGCTCCTTCGGTCCTCCGATCGTTGTCAGAAGTAAGTTGGCCGCAGTGTTATCACTCATGGTTATGGC |
| | AGCACTGCATAATTCTCTTACTGTCATGCCATCCGTAAGATGCTTTTCTGTGACTGGTGAGTACTCAACCAA |
| | GTCATTCTGAGAATAGTGTATGCGGCGACCGAGTTGCTCTTGCCCGGCGTCAATACGGGATAATACCGCG |
| | CCACATAGCAGAACTTTAAAAGTGCTCATCATTGGAAAACGTTCTTCGGGGCGAAAACTCTCAAGGATCTT |
| | ACCGCTGTTGAGATCCAGTTCGATGTAACCCACTCGTGCACCCAACTGATCTTCAGCATCTTTTACTTTCAC |
| | CAGCGTTTCTGGGTGAGCAAAAACAGGAAGGCAAAATGCCGCAAAAAAGGGAATAAGGGCGACACGGA |
| | AATGTTGAATACTCATACTCTTCCTTTTTCAATATTATTGAAGCATTTATCAGGGTTATTGTCTCATGAGCGG |
| | ATACATATTTGAATGTATTTAGAAAAATAAACAAATAGGGGTTCCGCGCACATTTCCCCGAAAAGTGCCAC |
| | CTGGGTCGACATTGATTATTGA |
| DHFR from | Optimized from E. coli codon usage |
| NEBExpress | ATGATCAGTCTGATTGCGGCGTTAGCGGTAGATCGCGTTATCGGCATGGAAAACGCCATGCCGTGGAACC |
| DHFRControl | TGCCTGCCGATCTCGCCTGGTTTAAACGCAACACCTTAAATAAACCCGTGATTATGGGCCGCCATACCTGG |
| Plasmid | GAATCAATCGGTCGTCCGTTGCCAGGACGCAAAAATATTATCCTCAGCAGTCAACCGGGTACGGACGATC |
| | GCGTAACGTGGGTGAAGTCGGTGGATGAAGCCATCGCGGCGTGTGGTGACGTACCAGAAATCATGGTGA |
| | TTGGCGGCGGTCGCGTTTATGAACAGTTCTTGCCAAAAGCGCAAAAACTGTATCTGACGCATATCGACGC |
| | AGAAGTGGAAGGCGACACCCATTTCCCGGATTACGAGCCGGATGACTGGGAATCGGTATTCAGCGAATTC |
| | CACGATGCTGATGCGCAGAACTCTCACAGCTATTGCTTTGAGATTCTGGAGCGGCGGTAA |

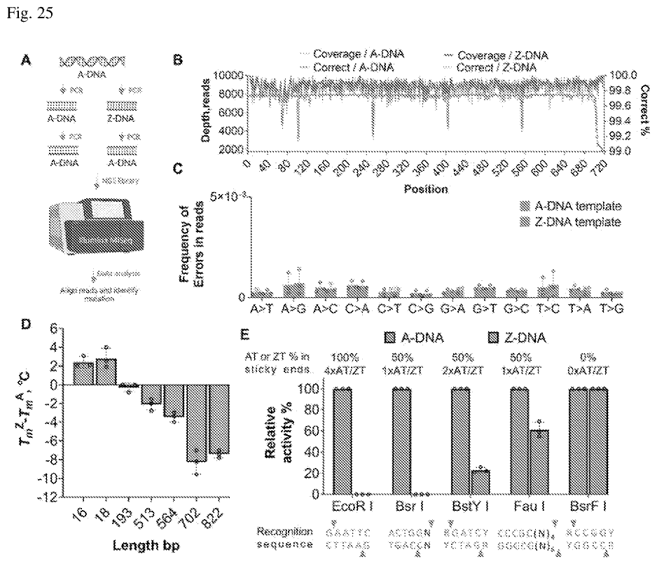

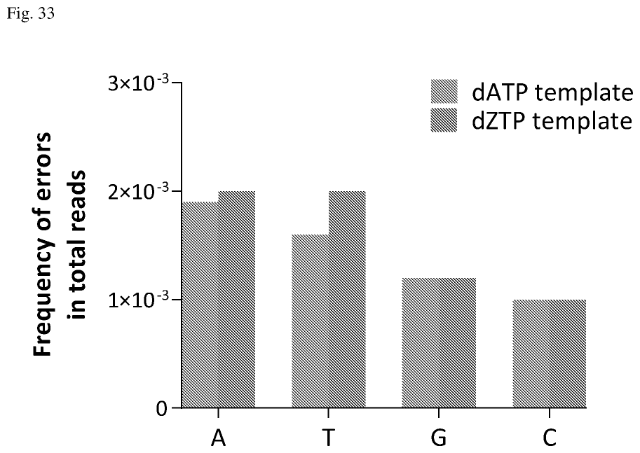

Compared with standard reactions, Z-substitutions achieved 60-100% relative yield of the intended band (FIG. 32A). We then conducted an analysis of the impact of GC % within the PCR amplicon on the PCR yield. As illustrated in FIG. 32B-C, we observed that an increase in GC % resulted in an increased PCR yield, although some outliers were also noted. Next, we evaluated whether Z-substitutions in the DNA1 template would lead to more mutations than adenine during PCR amplification. 720 bp PCR amplicons were amplified from A-DNA or Z-DNA templates and sequenced using an Illumina next generation sequencing (NGS) platform (FIG. 25A) (20, 21). We obtained 1803 to 7697 reads for each position of all PCR samples. Sequenced bases were compared to the reference to identify substitution errors. The average percentage of correct reads for each position ranged from 99.82% to 99.84% (FIG. 25B). Then we analyzed 12 types of base mutations, and results indicated no significant differences between the error rates of PCR products from A-DNA and Z-DNA (FIG. 25C). The further summarization of base mutation errors maintained that there were no significant differences observed between the two templates (FIG. 33). These results indicate that the non-Watson-Crick Z-T pair did not cause negative impacts on the accuracy of in vitro replication of the DNA1 template.

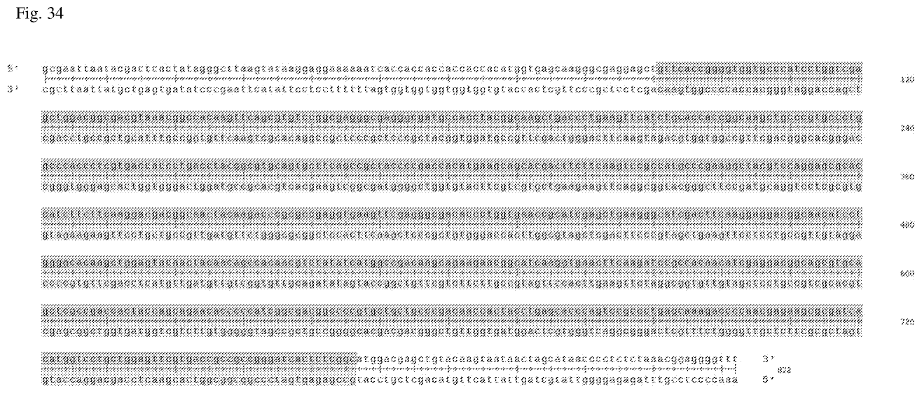

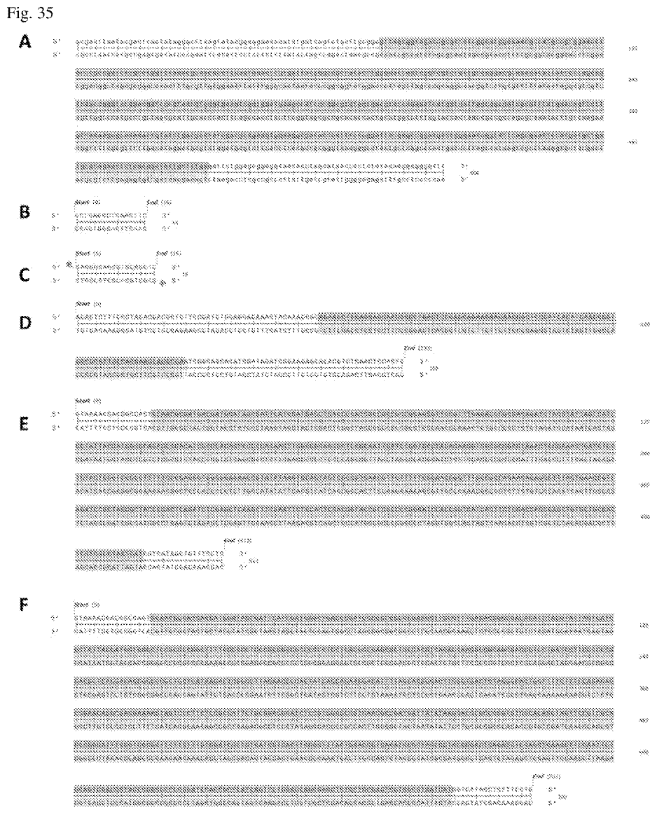

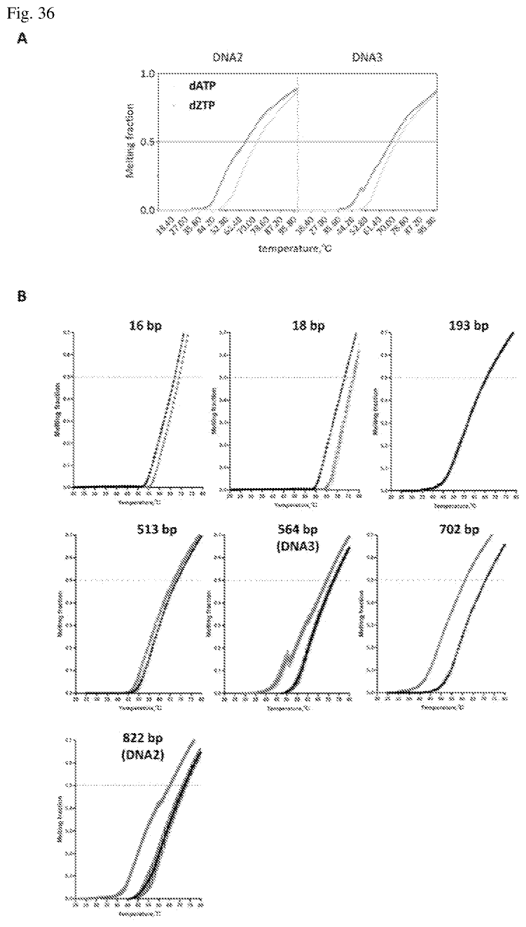

One physical property of DNA, melting temperature (Tₘ), represents the thermodynamic stability of a DNA double helix (22). Compared with the canonical A-T pairing, the Z-T pair possesses an extra hydrogen bond making it more similar to a G-C pair in regard to its Tₘ and minor groove width (23, 24). Other works found that short oligonucleotides less than 160 bp exhibited increases in Tₘ when incorporated with Z-bases (9, 25-27). Because of this, we investigated the effect of Z-substitutions on the Tₘ of longer DNAs. We first designed two additional artificial templates, DNA2 with length of 822 bp carrying 39% AT (FIG. 34) and DNA3 with length of 564 bp carrying 48% AT (FIG. 35A). Then we detected melting curves of the two natural and Z-substituted DNA templates. Surprisingly, the Tₘ of both templates decreased after Z-substitution. Specifically, the Tₘ of Z-DNA decreased 7.4° C. from 72.4° C. to 65° C. for DNA2 (FIG. 36A). This finding contrasts results from short dsDNAs in previous works. This discrepancy might be because the longer Z-DNA strands used in our research fail to keep a stable natural structure due to the presence of too many Z-T base pairs (28). We further evaluated another set of five DNA sequences with various lengths (FIG. 35B-F). The results revealed an obvious correlation between the length and the Tₘ of the DNAs tested. For sequences with a length of less than 20 base pairs, the Tₘ of dsDNA with Z substitutions was 2.4-2.8° C. higher than that of the standard. Conversely, for sequences longer than 500 base pairs, Tₘ decreased 2.1-8.3° C. after Z substitution (FIG. 25D and FIG. 36B).

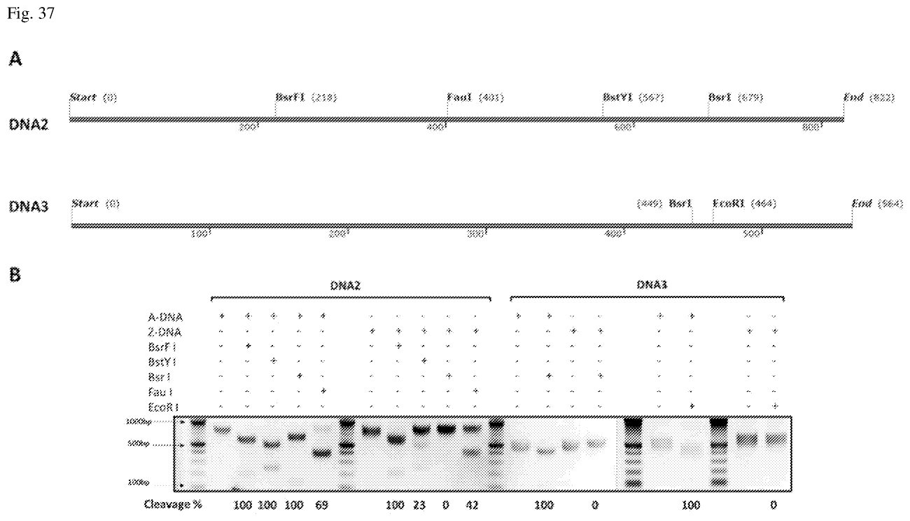

Z-substituted DNA has demonstrated resistance to digestion by REs, including EcoRI, with recognition sites containing one or more As (5, 11). To investigate the resistance characteristics of Z-DNA to REs with recognition sites containing one or more As or Zs, we chose EcoRI and four other REs (BsrFI, BstYI, BsrI and FauI) that were not tested in previous studies. BsrFI, BstYI, and EcoRI belong to the class of orthodox REs, while Bsrl and FauI belong to IIS REs. These enzymes can generate sticky ends containing A-T pairs from 0 to 100%. Additionally, Type II orthodox REs and Type IIS REs can recognize asymmetric DNA sequences and cleave either inside or outside their recognition sequence, respectively (29). We observed that BsrFI showed 100% relative activity on DNA sticky ends without Z-T base pairs. Conversely, EcoRI and BsrI were completely blocked, and the relative activity of BstYI and FauI decreased by 77% and 39%, respectively, when Z-T base pairs were introduced into the sticky ends. This suggests that the presence of Z-T in both the recognition sequence and the sticky ends weakens the activity of standard REs on Z-DNA, with the latter having a greater impact (FIG. 25E and FIG. 37). Both the improved base pair stability with the extra H-bond and the formation change of the groove both contributed to RE resistance in Z-substituted DNA strands. We concluded that the introduction of Z-bases into DNA not only leads to significant changes in the physical properties of DNA, but also further affects or even blocks the action of certain sequence-dependent enzyme systems. Interestingly, there are also some biological components that can be compatible with Z-DNA.

### Example 9: DNA Written by ZTGC can be Decoded to Specific Genetic Information in Mammalian Cells

Standard living systems can decode ATGC-DNA and output RNA and proteins according to the central dogma of molecular biology (30). Though it was reported that T7 RNA polymerase and human RNA polymerase II activity could be strongly blocked up to 92% by introducing a single Z-substitution in the region between promoter and coding sequence of standard plasmid DNA (13), it remains unknown whether Z-DNA at the gene-scale or higher levels can still be transcribed and translated into proteins. We reasoned that Z-DNA may be compatible with decoding systems since the Tₘ of Z-DNA changes at various lengths, and further evaluated whether different living systems can read and output genetic information from Z-DNA.

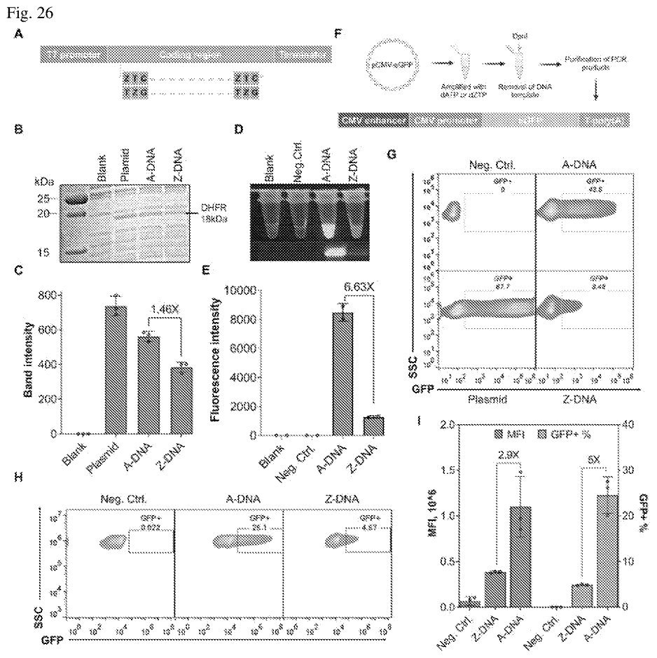

First, we reprogrammed the coding region of genes with ZTGC nucleotides (FIG. 26A). Considering that the size of the coding region may affect expression efficiency, we chose the proteins dihydrofolate reductase (DHFR) and enhanced green fluorescent protein (eGFP) (Tables 9 and 10), which have molecular weights of 18 kDa and 27 kDa, respectively.

TABLE 10
eGFP coding sequence for E.coli.

| Name | pIDT-eGFP, eGFP coding sequence optimized for E.coli |
|---|---|
| Sequence | TCGCGCGTTTCGGTGATGACGGTGAAAACCTCTGACACATGCAGCTCCCGGAGACGGTCACAGCTTGTC |
| | TGTAAGCGGATGCCGGGAGCAGACAAGCCCGTCAGGGCGCGTCAGCGGGTGTTGGCGGGTGTCGGGG |
| | CTGGCTTAACTATGCGGCATCAGAGCAGATTGTACTGAGAGTGCACCAAATGCGGTGTGAAATACCGCA |
| | CAGATGCGTAAGGAGAAAATACCGCATCAGGCGCCATTCGCCATTCAGGCTGCGCAACTGTTGGGAAG |
| | GGCGATCGGTGCGGGCCTCATCGCTATTACGCCAGCTGGCGAAAGGGGGATGTGCTGCAAGGCGATTA |
| | AGTTGGGTAACGCCAGGGTTTTCCCAGTCACGACGTTGTAAAACGACGGCCAGTGCAACGCGATGACG |
| | ATGGATAGCGATTCATCGATGAGCTGACCCGATCGCCGCCGCCGGAGGGTTGCGTTTGAGACGGGCGA |
| | CAGATATGGTGTCTAAGGGAGAAGAACTGTTTACCGGGGTAGTGCCCATTCTTGTCGAGCTTGACGGT |
| | GACGTAAATGGCCATAAGTTCTCAGTTTCTGGAGAGGGCGAGGGAGACGCTACGTACGGGAAGTTG |
| | ACACTGAAATTCATTTGCACGACGGGTAAATTGCCTGTGCCTTGGCCCACCTTAGTCACAACATTAACG |
| | TACGGGGTGCAGTGCTTCAGTCGCTACCCAGATCACATGAAGCAACACGACTTCTTCAAATCAGCCAT |
| | GCCTGAAGGATACGTGCAGGAACGTACAATCTTCTTCAAGGATGACGGCAATTACAAGACCCGTGCG |
| | GAAGTTAAATTTGAGGGCGACACACTTGTGAATCGCATCGAATTGAAAGGTATTGATTTCAAAGAAG |
| | ATGGGAATATCCTTGGGCATAAGTTGGAATATAACTACAACAGTCACAATGTTTACATCATGGCAGAT |
| | AAGCAGAAAAACGGTATTAAGGTAAATTTCAAAATTCGCCATAACATTGAGGATGGTTCGGTTCAACT |
| | TGCCGATCACTACCAGCAAAACACGCCTATTGGGGATGGACCAGTTCTTCTGCCCGATAATCATTACCT |
| | GAGCACGCAATCGGCTTTGTCAAAAGACCCAAACGAAAAACGTGACCATATGGTCTTGCTTGAGTTCG |
| | TAACGGCCGCCGGTATTACGCTTGGTATGGACGAATTATATAAGTGAATCAGTTCTGGACCAGCGAGC |
| | TGTGCTGCGACTCGTGGCGTAATCATGGTCATAGCTGTTTCCTGTGTGAAATTGTTATCCGCTCACAATT |
| | CCACACAACATACGAGCCGGAAGCATAAAGTGTAAAGCCTGGGGTGCCTAATGAGTGAGCTAACTCAC |
| | ATTAATTGCGTTGCGCTCACTGCCCGCTTTCCAGTCGGGAAACCTGTCGTGCCAGCTGCATTAATGAATC |
| | GGCCAACGCGCGGGGAGAGGCGGTTTGCGTATTGGGCGCTCTTCCGCTTCCTCGCTCACTGACTCGCTG |
| | CGCTCGGTCGTTCGGCTGCGGCGAGCGGTATCAGCTCACTCAAAGGCGGTAATACGGTTATCCACAGAA |
| | TCAGGGGATAACGCAGGAAAGAACATGTGAGCAAAAGGCCAGCAAAAGGCCAGGAACCGTAAAAAGG |
| | CCGCGTTGCTGGCGTTTTTCCATAGGCTCCGCCCCCCTGACGAGCATCACAAAAATCGACGCTCAAGTCA |
| | GAGGTGGCGAAACCCGACAGGACTATAAAGATACCAGGCGTTTCCCCCTGGAAGCTCCCTCGTGCGCTC |
| | TCCTGTTCCGACCCTGTCGCTTACCGGATACCTGTCCGCCTTTCTCCCTTCGGGAAGCGTGGCGCTTTCTC |
| | ATAGCTCACGCTGTAGGTATCTCAGTTCGGTGTAGGTCGTTCGCTCCAAGCTGGGCTGTGTGCACGAAC |
| | CCCCCGTTCAGCCCGACCGCTGCGCCTTATCCGGTAACTATCGTCTTGAGTCCAACCCGGTAAGACACGA |
| | CTTATCGCCACTGGCAGCAGCCACTGGTAACAGGATTAGCAGAGCGAGGTATGTAGGCGGTGCTACAG |
| | AGTTCTTGAAGTGGTGGCCTAACTACGGCTACACTAGAAGAACAGTATTTGGTATCTGCGCTCTGCTGA |
| | AGCCAGTTACCTTCGGAAAAAGAGTTGGTAGCTCTTGATCCGGCAAACAAACCACCGCTGGTAGCGGTG |
| | GTTTTTTTGTTTGCAAGCAGCAGATTACGCGCAGAAAAAAAGGATCTCAAGAAGATCCTTTGATCTTTTC |
| | TACGGGGTCTGACGCTCAGTGGAACGAAAACTCACGTTAAGGGATTTTGGTCATGAGATTATCAAAAAG |
| | GATCTTCACCTAGATCCTTTTAAATTAAAAATGAAGTTTTAAATCAATCTAAAGTATATATGAGTAAACTT |
| | GGTCTGACAGTTACCAATGCTTAATCAGTGAGGCACCTATCTCAGCGATCTGTCTATTTCGTTCATCCATA |
| | GTTGCCTGACTCCCCGTCGTGTAGATAACTACGATACGGGAGGGCTTACCATCTGGCCCCAGTGCTGCA |
| | ATGATACCGCGAGACCCACGCTCACCGGCTCCAGATTTATCAGCAATAAACCAGCCAGCCGGAAGGGCC |
| | GAGCGCAGAAGTGGTCCTGCAACTTTATCCGCCTCCATCCAGTCTATTAATTGTTGCCGGGAAGCTAGA |
| | GTAAGTAGTTCGCCAGTTAATAGTTTGCGCAACGTTGTTGCCATTGCTACAGGCATCGTGGTGTCACGCT |
| | CGTCGTTTGGTATGGCTTCATTCAGCTCCGGTTCCCAACGATCAAGGCGAGTTACATGATCCCCCATGTT |
| | GTGCAAAAAAGCGGTTAGCTCCTTCGGTCCTCCGATCGTTGTCAGAAGTAAGTTGGCCGCAGTGTTATC |
| | ACTCATGGTTATGGCAGCACTGCATAATTCTCTTACTGTCATGCCATCCGTAAGATGCTTTTCTGTGACTG |
| | GTGAGTACTCAACCAAGTCATTCTGAGAATAGTGTATGCGGCGACCGAGTTGCTCTTGCCCGGCGTCAA |
| | TACGGGATAATACCGCGCCACATAGCAGAACTTTAAAAGTGCTCATCATTGGAAAACGTTCTTCGGGGC |
| | GAAAACTCTCAAGGATCTTACCGCTGTTGAGATCCAGTTCGATGTAACCCACTCGTGCACCCAACTGATC |
| | TTCAGCATCTTTTACTTTCACCAGCGTTTCTGGGTGAGCAAAAACAGGAAGGCAAAATGCCGCAAAAAA |
| | GGGAATAAGGGCGACACGGAAATGTTGAATACTCATACTCTACCTTTTTCAATATTATTGAAGCATTTAT |
| | CAGGGTTATTGTCTCATGAGCGGATACATATTTGAATGTATTTAGAAAAATAAACAAATAGGGGTTCCG |
| | CGCACATTTCCCCGAAAAGTGCCACCTGACGTCTAAGAAACCATTATTATCATGACATTAACCTATAAAA |
| | ATAGGCGTATCACGAGGCCCTTTCGTC |
| Note | eGFP orf (E.coli codon optimized) |

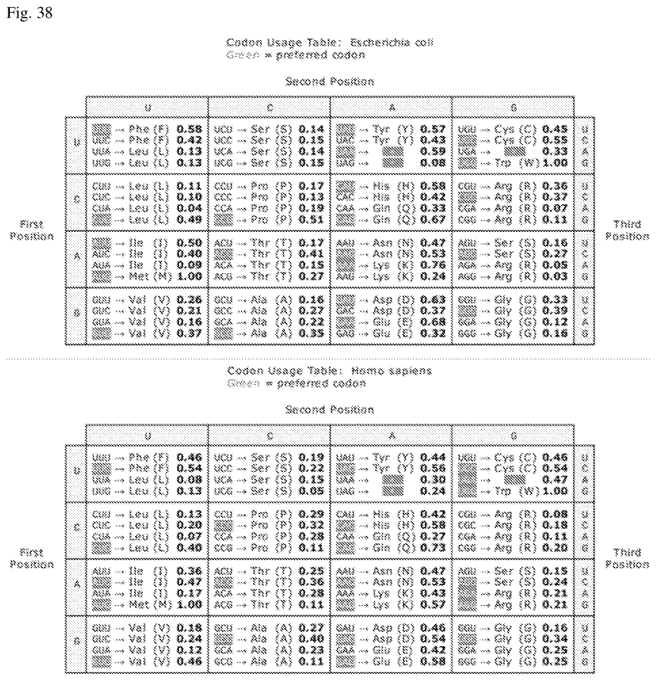

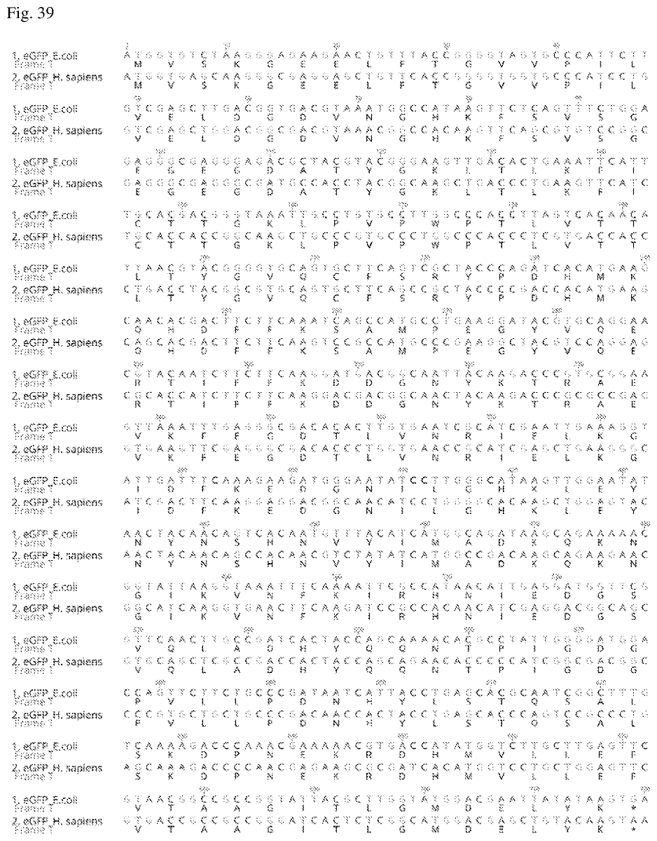

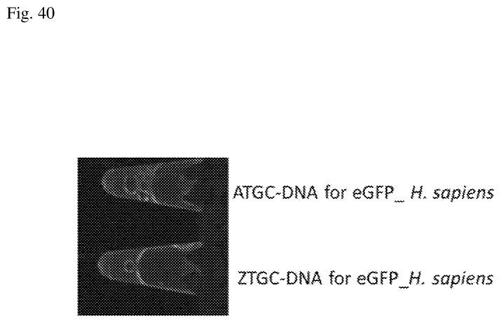

The two coding sequences were optimized based on codon usage frequency in Escherichia coli and were placed under the control of a T7 promoter. The experiment was carried out in a cell-free system extracted from E. coli. Both DHFR and eGFP were successfully expressed using Z-DNA templates (FIG. 26B-E), though the Z-DNA protein yield for DHFR and eGFP was significantly reduced when compared to A-DNA templates. Specifically, the yield dropped by 35.4% for DHFR and a substantial 84.9% for eGFP. These results indicate that the transcription and translation system of E. coli is compatible with Z-DNA coding sequences, and outputs the correct genetic information. Different species are known to have different codon preferences. For example, Homo sapiens prefer GC rich codons compared to E. coli (FIG. 38) (31). Next, we investigated whether Z-DNA templates with codons optimized for eukaryotes (FIG. 39 and Tables 11 and 12) could express proteins in prokaryotes. Neither Z-DNA nor standard DNA templates carrying coding sequences optimized for H. sapiens led to the eGFP expression (FIG. 40). These results demonstrated that codon usage bias still exists for protein expression using Z-DNA templates of eGFP.

TABLE 11
DNA templates used in cell free expression assay.

| | | |
|---|---|---|
| 1 | Name | pure express PCR eGFP Template-eGFP Human |
| | Sequence | GCGAATTAATACGACTCACTATAGGGCTTAAGTATAAGGAGGAAAAAATATGGTGAGCAAGGGCG |
| | | AGGAGCTGTTCACCGGGGTGGTGCCCATCCTGGTCGAGCTGGACGGCGACGTAAACGGCCACAAGT |
| | | TCAGCGTGTCCGGCGAGGGCGAGGGCGATGCCACCTACGGCAAGCTGACCCTGAAGTTCATCTGCA |
| | | CCACCGGCAAGCTGCCCGTGCCCTGGCCCACCCTCGTGACCACCCTGACCTACGGCGTGCAGTGCTT |
| | | CAGCCGCTACCCCGACCACATGAAGCAGCACGACTTCTTCAAGTCCGCCATGCCCGAAGGCTACGTC |
| | | CAGGAGCGCACCATCTTCTTCAAGGACGACGGCAACTACAAGACCCGCGCCGAGGTGAAGTTCGAG |
| | | GGCGACACCCTGGTGAACCGCATCGAGCTGAAGGGCATCGACTTCAAGGAGGACGGCAACATCCTG |
| | | GGGCACAAGCTGGAGTACAACTACAACAGCCACAACGTCTATATCATGGCCGACAAGCAGAAGAAC |
| | | GGCATCAAGGTGAACTTCAAGATCCGCCACAACATCGAGGACGGCAGCGTGCAGCTCGCCGACCAC |
| | | TACCAGCAGAACACCCCCATCGGCGACGGCCCCGTGCTGCTGCCCGACAACCACTACCTGAGCACCC |
| | | AGTCCGCCCTGAGCAAAGACCCCAACGAGAAGCGCGATCACATGGTCCTGCTGGAGTTCGTGACCG |
| | | CCGCCGGGATCACTCTCGGCATGGACGAGCTGTACAAGTAATAACTAGCATAACCCCTCTCTAAACG |
| | | GAGGGGTTT |
| | Note | T7 promoter |
| | | G050 and G051 primers sequence |
| | | Cas9 target |
| 2 | Name | pure express PCR eGFP Template-eGFP E. coli |
| | Sequence | GCGAATTAATACGACTCACTATAGGGCTTAAGTATAAGGAGGAAAAAATATGGTGTCTAAGGGAG |
| | | AAGAACTGTTTACCGGGGTAGTGCCCATTCTTGTCGAGCTTGACGGTGACGTAAATGGCCATAAGTT |
| | | CTCAGTTTCTGGAGAGGGCGAGGGAGACGCTACGTACGGGAAGTTGACACTGAAATTCATTTGCAC |
| | | GACGGGTAAATTGCCTGTGCCTTGGCCCACCTTAGTCACAACATTAACGTACGGGGTGCAGTGCTTC |
| | | AGTCGCTACCCAGATCACATGAAGCAACACGACTTCTTCAAATCAGCCATGCCTGAAGGATACGTGC |
| | | AGGAACGTACAATCTTCTTCAAGGATGACGGCAATTACAAGACCCGTGCGGAAGTTAAATTTGAGG |
| | | GCGACACACTTGTGAATCGCATCGAATTGAAAGGTATTGATTTCAAAGAAGATGGGAATATCCTTGG |
| | | GCATAAGTTGGAATATAACTACAACAGTCACAATGTTTACATCATGGCAGATAAGCAGAAAAACGGT |
| | | ATTAAGGTAAATTTCAAAATTCGCCATAACATTGAGGATGGTTCGGTTCAACTTGCCGATCACTACCA |
| | | GCAAAACACGCCTATTGGGGATGGACCAGTTCTTCTGCCCGATAATCATTACCTGAGCACGCAATCG |
| | | GCTTTGTCAAAAGACCCAAACGAAAAACGTGACCATATGGTCTTGCTTGAGTTCGTAACGGCCGCCG |
| | | GTATTACGCTTGGTATGGACGAATTATATAAGTGATAACTAGCATAACCCCTCTCTAAACGGAGGGG |
| | | TTT |
| | Note | T7 promoter |
| | | G063 and G064 primers sequence |
| 3 | Name | pure express PCR DHFR Control Template-dihydrofolate reductase (DHFR) |
| | Sequence | GCGAATTAATACGACTCACTATAGGGCTTAAGTATAAGGAGGAAAAAATATGATCAGTCTGATTGC |
| | | GGCGTTAGCGGTAGATCGCGTTATCGGCATGGAAAACGCCATGCCGTGGAACCTGCCTGCCGATCT |
| | | CGCCTGGTTTAAACGCAACACCTTAAATAAACCCGTGATTATGGGCCGCCATACCTGGGAATCAATC |
| | | GGTCGTCCGTTGCCAGGACGCAAAAATATTATCCTCAGCAGTCAACCGGGTACGGACGATCGCGTA |
| | | ACGTGGGTGAAGTCGGTGGATGAAGCCATCGCGGCGTGTGGTGACGTACCAGAAATCATGGTGAT |
| | | TGGCGGCGGTCGCGTTTATGAACAGTTCTTGCCAAAAGCGCAAAAACTGTATCTGACGCATATCGAC |
| | | GCAGAAGTGGAAGGCGACACCCATTTCCCGGATTACGAGCCGGATGACTGGGAATCGGTATTCAGC |
| | | GAATTCCACGATGCTGATGCGCAGAACTCTCACAGCTATTGCTTTGAGATTCTGGAGCGGCGGTAAT |
| | | AACTAGCATAACCCCTCTCTAAACGGAGGGGTTT |
| | Note | T7 promoter |
| | | G052 and G053 primers sequence |

TABLE 12
pMRNA-eGFP plasmid sequence.

| Name | pMRNA-eGFP |
|---|---|
| Sequence | CCCGTGTAAAACGACGGCCAGTTTATCTAGTCAGCTTGATTCTAGCTGATCGTGGACCGGAAGGTGAGC |
| | CAGTGAGTTGATTGCAGTCCAGTTACGCTGGAGTCTGAGGCTCGTCCTGAATGATATGCGACCGCCGGA |
| | GGGTTGCGTTTGAGACGGGCGACAGATCGACACTGCTCGATCCGCTCGCACCACTAGTTCTAGAGGTAC |
| | CCCTGCAGGTTGGACCCTCGTACAGAAGCTAATACGACTCACTATAGGGAAATAAGAGAGAAAAGAAG |
| | AGTAAGAAGAAATATAAGAGAATTCGCCGCCACCATGGTGAGCAAGGGCGAGGAGCTGTTCACCGGG |
| | GTGGTGCCCATCCTGGTCGAGCTGGACGGCGACGTAAACGGCCACAAGTTCAGCGTGTCCGGCGAGG |
| | GCGAGGGCGATGCCACCTACGGCAAGCTGACCCTGAAGTTCATCTGCACCACCGGCAAGCTGCCCGTG |
| | CCCTGGCCCACCCTCGTGACCACCCTGACCTACGGCGTGCAGTGCTTCAGCCGCTACCCCGACCACATG |
| | AAGCAGCACGACTTCTTCAAGTCCGCCATGCCCGAAGGCTACGTCCAGGAGCGCACCATCTTCTTCAA |
| | GGACGACGGCAACTACAAGACCCGCGCCGAGGTGAAGTTCGAGGGCGACACCCTGGTGAACCGCATC |
| | GAGCTGAAGGGCATCGACTTCAAGGAGGACGGCAACATCCTGGGGCACAAGCTGGAGTACAACTAC |
| | AACAGCCACAACGTCTATATCATGGCCGACAAGCAGAAGAACGGCATCAAGGTGAACTTCAAGATCC |
| | GCCACAACATCGAGGACGGCAGCGTGCAGCTCGCCGACCACTACCAGCAGAACACCCCCATCGGCGA |
| | CGGCCCCGTGCTGCTGCCCGACAACCACTACCTGAGCACCCAGTCCGCCCTGAGCAAAGACCCCAACG |
| | AGAAGCGCGATCACATGGTCCTGCTGGAGTTCGTGACCGCCGCCGGGATCACTCTCGGCATGGACGA |
| | GCTGTACAAGTAAGGATCCGCTGCCTTCTGCGGGGCTTGCCTTCTGGCCATGCCCTTCTTCTCTCCCTTGC |
| | ACCTGTACCTCTTGGTCTTTGAATAAAGCCTGAGTAGGAAGAATATTAAAAAAAAAAAAAAAAAAAAA |
| | AAAAAAAAAAAAAAAAAAAAAAAATTAATTAACATATGGCGGCCGCTCTAGAACTAGTGTCGACGCG |
| | GATCGACGAGAGCAGCGCGACTGGATCAGTTCTGGACGAGCGAGCTGTCGTCCGACCCGTGATCTTAC |
| | GGCATTATACGTATGATCGGTCCACGATCAGCTAGATTATCTAGTCAGCTTGATGTCATAGCTGTTTCCT |
| | GAGGCTCAATACTGACCATTTAAATCATACCTGACCTCCATAGCAGAAAGTCAAAAGCCTCCGACCGGA |
| | GGCTTTTGACTTGATCGGCACGTAAGAGGTTCCAACTTTCACCATAATGAAATAAGATCACTACCGGGC |
| | GTATTTTTTGAGTTATCGAGATTTTCAGGAGCTAAGGAAGCTAAAATGAGTATTCAACATTTCCGTGTCG |
| | CCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCACGAGTGGGTTACATCGAACTGGATCTCAACAGCGGTAAGATCCTT |
| | GAGAGTTTACGCCCCGAAGAACGTTTTCCAATGATGAGCACTTTTAAAGTTCTGCTATGTGGCGCGGTAT |
| | TATCCCGTATTGACGCCGGGCAAGAGCAACTCGGTCGCCGCATACACTATTCTCAGAATGACTTGGTTG |
| | AGTACTCACCAGTCACAGAAAAGCATCTCACGGATGGCATGACAGTAAGAGAATTATGCAGTGCTGCCA |
| | TAACCATGAGTGATAACACTGCGGCCAACTTACTTCTGGCAACGATCGGAGGACCGAAGGAGCTAACC |
| | GCTTTTTTGCACAACATGGGGGATCATGTAACTCGCCTTGATCGTTGGGAACCGGAGCTGAATGAAGCC |
| | ATACCAAACGACGAGCGTGACACCACGATGCCTGTAGCAATGGCAACAACGTTGCGCAAACTATTAACT |
| | GGCGAACTACTTACTCTAGCTTCCCGGCAACAATTAATAGACTGGATGGAGGCGGATAAAGTTGCAGGA |
| | TCACTTCTGCGCTCGGCCCTCCCGGCTGGCTGGTTTATTGCTGATAAATCTGGAGCCGGTGAGCGTGGG |
| | TCTCGCGGTATCATTGCAGCACTGGGGCCAGATGGTAAGCCCTCCCGCATCGTAGTTATCTACACGACG |
| | GGGAGTCAGGCAACTATGGATGAACGAAATAGACAGATCGCTGAGATAGGTGCCTCACTGATTAAGCA |
| | TTGGTAATGAGGGCCCAAATGTAATCACCTGGCTCACCTTCGGGTGGGCCTTTCTTGAGGACCTAAATGT |
| | AATCACCTGGCTCACCTTCGGGTGGGCCTTTCTTGAGGACCTAAATGTAATCACCTGGCTCACCTTCGGG |
| | TGGGCCTTTCTTGAGGACCTAAATGTAATCACCTGGCTCACCTTCGGGTGGGCCTTTCTTGAGGACCTAA |
| | ATGTAATCACCTGGCTCACCTTCGGGTGGGCCTTTCTGCGTTGCTGGCGTTTTTCCATAGGCTCCGCCCCC |
| | CTGACGAGCATCACAAAAATCGATGCTCAAGTCAGAGGTGGCGAAACCCGACAGGACTATAAAGATAC |
| | CAGGCGTTTCCCCCTGGAAGCTCCCTCGTGCGCTCTCCTGTTCCGACCCTGCCGCTTACCGGATACCTGTC |
| | CGCCTTTCTCCCTTCGGGAAGCGTGGCGCTTTCTCATAGCTCACGCTGTAGGTATCTCAGTTCGGTGTAG |
| | GTCGTTCGCTCCAAGCTGGGCTGTGTGCACGAACCCCCCGTTCAGCCCGACCGCTGCGCCTTATCCGGTA |
| | ACTATCGTCTTGAGTCCAACCCGGTAAGACACGACTTATCGCCACTGGCAGCAGCCACTGGTAACAGGA |
| | TTAGCAGAGCGAGGTATGTAGGCGGTGCTACAGAGTTCTTGAAGTGGTGGCCTAACTACGGCTACACTA |
| | GAAGAACAGTATTTGGTATCTGCGCTCTGCTGAAGCCAGTTACCTCGGAAAAAGAGTTGGTAGCTCTTG |
| | ATCCGGCAAACAAACCACCGCTGGTAGCGGTGGTTTTTTTGTTTGCAAGCAGCAGATTACGCGCAGAAA |
| | AAAAGGATCTCAAGAAGATCCTTTGATTTTCTACCGAAGAAAGGCCCA |
| Note | 5′-UTR |
| | Kozak sequence |
| | eGFP orf (Human codon optimized) |
| | Cas12a target region |
| | Cas9 target region |
| | 3′-UTR |
| | PolyA |
| | T7 promoter |

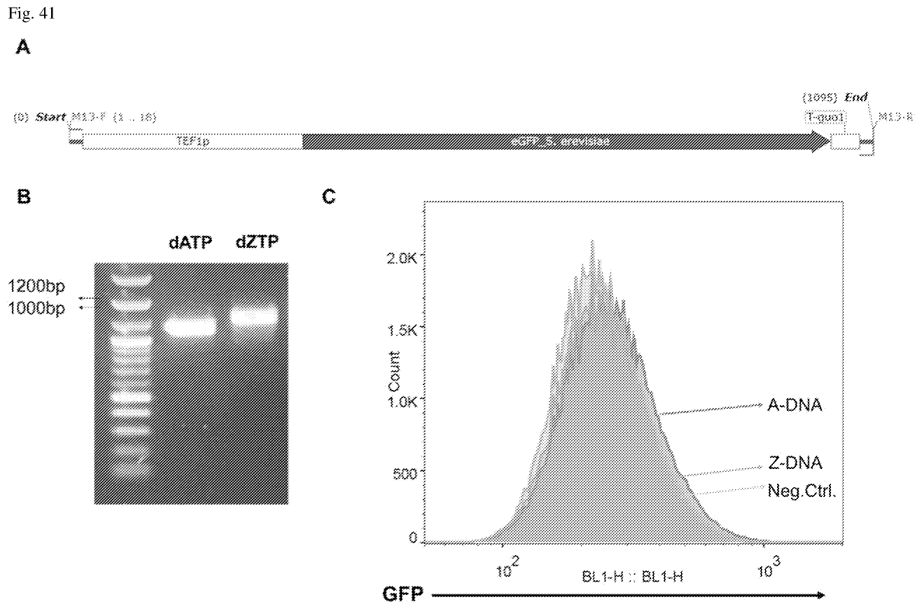

Then we replaced all A-bases located in the promoter, coding box, and terminator region of the DNA construct with Z-bases to test its compatibility in living systems. DNA constructs were assembled in the promoter-gene-terminator architecture. For single-cell eukaryotes Saccharomyces cerevisiae, a TEFlp promoter with 300 bp length and 71% AT content, a gene eGFP optimized for S. cerevisiae with 720 bp length and 54% AT content, and a CYC1t terminator with 39 bp length and 77% AT content were selected for cassette assembly (FIG. 41A-B and Table 13) (32). Excitingly, fluorescent cells were detected in the S. cerevisiae BY4742 cell population transformed with the Z-DNA cassette (FIG. 41B).

TABLE 13
Cassette of TEF1p-eGFP_{SC}-CYC1t for Saccharomyces cerevisiae.

| Name | TEF1p-eGFP_{SC}-CYC1t inserted into pIDT |
|---|---|
| Sequence | TGTAAAACGACGGCCAGTTCTTCCTCTAGGGTGTCGTTAATTACCCGTACTAAAGGTTTGGAAAAGAAA |
| | AAAGAGACCGCCTCGTTTCTTTTTCTTCGTCGAAAAAGGCAATAAAAATTTTTATCACGTTTCTTTTTCTTG |
| | AAAATTTTTTTTTTTGATTTTTTTCTCTTTCGATGACCTCCCATTGATATTTAAGTTAATAAACGGTCTTCAA |
| | TTTCTCAAGTTTCAGTTTCATTTTTCTTGTTCTATTACAACTTTTTTTACTTCTTGCTCATTAGAAAGAAAGC |
| | ATAGCAATCTAATCTAAGTTTTAATTACAAAATGGTTTCCAAAGGAGAAGAGTTGTTTACTGGGGTGGTC |
| | CCAATTCTTGTGGAATTGGATGGCGACGTTAATGGGCATAAATTCAGCGTGAGTGGTGAAGGTGAAGG |
| | AGACGCCACTTACGGCAAGCTAACCCTAAAATTTATATGTACGACTGGTAAACTTCCTGTACCATGGCCT |
| | ACTCTTGTAACTACATTAACGTATGGAGTACAATGCTTTTCTAGGTATCCGGATCATATGAAGCAGCATG |
| | ACTTTTTCAAGTCTGCGATGCCGGAAGGGTATGTGCAGGAAAGGACTATTTTCTTCAAAGACGATGGTA |
| | ACTATAAGACTAGGGCCGAGGTCAAGTTTGAGGGGGATACGCTTGTGAACAGGATCGAGCTGAAAGGT |
| | ATCGATTTTAAGGAGGATGGGAACATTCTTGGGCACAAGCTAGAGTATAATTATAATTCCCACAATGTTT |
| | ATATAATGGCCGATAAGCAAAAAAATGGCATTAAGGTGAATTTCAAGATTCGTCATAATATTGAAGACG |
| | GTTCAGTGCAACTAGCAGACCACTATCAGCAAAACACACCTATAGGAGATGGTCCTGTGTTACTACCCGA |
| | CAATCACTACTTGTCCACCCAGTCTGCTCTATCCAAAGATCCTAACGAGAAACGTGACCATATGGTCTTGT |
| | TAGAGTTCGTTACAGCGGCAGGCATAACCTTAGGTATGGACGAGTTATACAAATAATATATAACTGTCT |
| | AGAAATAAAGAGTATCATCTTTCAAAGGTCATAGCTGTTTCCTG |
| Note | Primer G097 |
| | TEF1p promoter |
| | eGFP |
| | terminator |
| | Primer G098 |

Next, we tested whether the Z-DNA could be decoded in human embryonic kidney (HEK) 293T cells. Genetic element cytomegalovirus (CMV) enhancer/promoter with 508 bp length and 50% AT content and a terminator with 207 bp and 53% AT content were used to control an eGFP gene optimized for H. sapiens with 720 bp and 38% AT (FIG. 26F and Table 14).

TABLE 14
pCMV-GFP plasmid.

| Name | pCMV-GFP, addgene #11153 |
|---|---|
| Sequence | CTAGTAATAGTAATCAATTACGGGGTCATTAGTTCATAGCCCATATATGGAGTTCCGCGTTACATAACTT |
| | ACGGTAAATGGCCCGCCTGGCTGACCGCCCAACGACCCCCGCCCATTGACGTCAATAATGACGTATGTTC |
| | CCATAGTAACGCCAATAGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGACGGTAAATGGCCCGCC |
| | TGGCATTATGCCCAGTACATGACCTTATGGGACTTTCCTACTTGGCAGTACATCTACGTATTAGTCATCGC |
| | TATTACCATGGTGATGCGGTTTTGGCAGTACATCAATGGGCGTGGATAGCGGTTTGACTCACGGGGAT |
| | TTCCAAGTCTCCACCCCATTGACGTCAATGGGAGTTTGTTTTGGCACCAAAATCAACGGGACTTTCCAA |
| | AATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCCGCTAGCGCTACCGGACTCAGATCTCGAGCTCAAGCTTC |
| | GAATTCTGCAGTCGACGGTACCGCGGGCCCGGGATCCACCGGTCGCCACCATGGTGAGCAAGGGCGA |
| | GGAGCTGTTCACCGGGGTGGTGCCCATCCTGGTCGAGCTGGACGGCGACGTAAACGGCCACAAGTTC |
| | AGCGTGTCCGGCGAGGGCGAGGGCGATGCCACCTACGGCAAGCTGACCCTGAAGTTCATCTGCACCA |
| | CCGGCAAGCTGCCCGTGCCCTGGCCCACCCTCGTGACCACCCTGACCTACGGCGTGCAGTGCTTCAGCC |
| | GCTACCCCGACCACATGAAGCAGCACGACTTCTTCAAGTCCGCCATGCCCGAAGGCTACGTCCAGGAG |
| | CGCACCATCTTCTTCAAGGACGACGGCAACTACAAGACCCGCGCCGAGGTGAAGTTCGAGGGCGACA |
| | CCCTGGTGAACCGCATCGAGCTGAAGGGCATCGACTTCAAGGAGGACGGCAACATCCTGGGGCACAA |
| | GCTGGAGTACAACTACAACAGCCACAACGTCTATATCATGGCCGACAAGCAGAAGAACGGCATCAAG |
| | GTGAACTTCAAGATCCGCCACAACATCGAGGACGGCAGCGTGCAGCTCGCCGACCACTACCAGCAGA |
| | ACACCCCCATCGGCGACGGCCCCGTGCTGCTGCCCGACAACCACTACCTGAGCACCCAGTCCGCCCTGA |
| | GCAAAGACCCCAACGAGAAGCGCGATCACATGGTCCTGCTGGAGTTCGTGACCGCCGCCGGGATCAC |
| | TCTCGGCATGGACGAGCTGTACAAGTAAAGCGGCCGCACTCCTCAGGTGCAGGCTGCCTATCAGAAGG |
| | TGGTGGCTGGTGTGGCCAATGCCCTGGCTCACAAATACCACTGAGATCTTTTTCCCTCTGCCAAAAATTA |
| | TGGGGACATCATGAAGCCCCTTGAGCATCTGACTTCTGGCTAATAAAGGAAATTTATTTTCATTGCAAT |
| | AGTGTGTTGGAATTTTTTGTGTCTCTCACTCGGAAGGACATATGGGAGGGCAAATCATTTAAAACATCA |
| | GAATGAGTATTTGGTTTAGAGTTTGGCAACATATGCCCATATGCTGGCTGCCATGAACAAAGGTTGGCT |
| | ATAAAGAGGTCATCAGTATATGAAACAGCCCCCTGCTGTCCATTCCTTATTCCATAGAAAAGCCTTGACT |
| | TGAGGTTAGATTTTTTTTATATTTTGTTTTGTGTTATTTTTTTCTTTAACATCCCTAAAATTTTCCTTACATG |
| | TTTTACTAGCCAGATTTTTCCTCCTCTCCTGACTACTCCCAGTCATAGCTGTCCCTCTTCTCTTATGGAGAT |
| | CCCTCGACCTGCAGCCCAAGCTTGGCGTAATCATGGTCATAGCTGTTTCCTGTGTGAAATTGTTATCCGC |
| | TCACAATTCCACACAACATACGAGCCGGAAGCATAAAGTGTAAAGCCTGGGGTGCCTAATGAGTGAGCT |
| | AACTCACATTAATTGCGTTGCGCTCACTGCCCGCTTTCCAGTCGGGAAACCTGTCGTGCCAGCGGATCCG |
| | CATCTCAATTAGTCAGCAACCATAGTCCCGCCCCTAACTCCGCCCATCCCGCCCCTAACTCCGCCCAGTTC |
| | CGCCCATTCTCCGCCCCATGGCTGACTAATTTTTTTTATTTATGCAGAGGCCGAGGCCGCCTCGGCCTCTG |
| | AGCTATTCCAGAAGTAGTGAGGAGGCTTTTTTGGAGGCCTAGGCTTTTGCAAAAAGCTAACTTGTTTATT |
| | GCAGCTTATAATGGTTACAAATAAAGCAATAGCATCACAAATTTCACAAATAAAGCATTTTTTTCACTGC |
| | ATTCTAGTTGTGGTTTGTCCAAACTCATCAATGTATCTTATCATGTCTGGATCCGCTGCATTAATGAATCG |
| | GCCAACGCGCGGGGAGAGGCGGTTTGCGTATTGGGCGCTCTTCCGCTTCCTCGCTCACTGACTCGCTGC |
| | GCTCGGTCGTTCGGCTGCGGCGAGCGGTATCAGCTCACTCAAAGGCGGTAATACGGTTATCCACAGAAT |
| | CAGGGGATAACGCAGGAAAGAACATGTGAGCAAAAGGCCAGCAAAAGGCCAGGAACCGTAAAAAGGC |
| | CGCGTTGCTGGCGTTTTTCCATAGGCTCCGCCCCCCTGACGAGCATCACAAAAATCGACGCTCAAGTCAG |
| | AGGTGGCGAAACCCGACAGGACTATAAAGATACCAGGCGTTTCCCCCTGGAAGCTCCCTCGTGCGCTCT |
| | CCTGTTCCGACCCTGCCGCTTACCGGATACCTGTCCGCCTTTCTCCCTTCGGGAAGCGTGGCGCTTTCTCA |
| | TAGCTCACGCTGTAGGTATCTCAGTTCGGTGTAGGTCGTTCGCTCCAAGCTGGGCTGTGTGCACGAACC |
| | CCCCGTTCAGCCCGACCGCTGCGCCTTATCCGGTAACTATCGTCTTGAGTCCAACCCGGTAAGACACGAC |
| | TTATCGCCACTGGCAGCAGCCACTGGTAACAGGATTAGCAGAGCGAGGTATGTAGGCGGTGCTACAGA |
| | GTTCTTGAAGTGGTGGCCTAACTACGGCTACACTAGAAGAACAGTATTTGGTATCTGCGCTCTGCTGAA |
| | GCCAGTTACCTTCGGAAAAAGAGTTGGTAGCTCTTGATCCGGCAAACAAACCACCGCTGGTAGCGGTGG |
| | TTTTTTTGTTTGCAAGCAGCAGATTACGCGCAGAAAAAAAGGATCTCAAGAAGATCCTTTGATCTTTTCT |
| | ACGGGGTCTGACGCTCAGTGGAACGAAAACTCACGTTAAGGGATTTTGGTCATGAGATTATCAAAAAG |
| | GATCTTCACCTAGATCCTTTTAAATTAAAAATGAAGTTTTAAATCAATCTAAAGTATATATGAGTAAACTT |
| | GGTCTGACAGTTACCAATGCTTAATCAGTGAGGCACCTATCTCAGCGATCTGTCTATTTCGTTCATCCATA |
| | GTTGCCTGACTCCCCGTCGTGTAGATAACTACGATACGGGAGGGCTTACCATCTGGCCCCAGTGCTGCA |
| | ATGATACCGCGAGACCCACGCTCACCGGCTCCAGATTTATCAGCAATAAACCAGCCAGCCGGAAGGGCC |
| | GAGCGCAGAAGTGGTCCTGCAACTTTATCCGCCTCCATCCAGTCTATTAATTGTTGCCGGGAAGCTAGA |
| | GTAAGTAGTTCGCCAGTTAATAGTTTGCGCAACGTTGTTGCCATTGCTACAGGCATCGTGGTGTCACGCT |
| | CGTCGTTTGGTATGGCTTCATTCAGCTCCGGTTCCCAACGATCAAGGCGAGTTACATGATCCCCCATGTT |
| | GTGCAAAAAAGCGGTTAGCTCCTTCGGTCCTCCGATCGTTGTCAGAAGTAAGTTGGCCGCAGTGTTATC |
| | ACTCATGGTTATGGCAGCACTGCATAATTCTCTTACTGTCATGCCATCCGTAAGATGCTTTTCTGTGACTG |
| | GTGAGTACTCAACCAAGTCATTCTGAGAATAGTGTATGCGGCGACCGAGTTGCTCTTGCCCGGCGTCAA |
| | TACGGGATAATACCGCGCCACATAGCAGAACTTTAAAAGTGCTCATCATTGGAAAACGTTCTTCGGGGC |
| | GAAAACTCTCAAGGATCTTACCGCTGTTGAGATCCAGTTCGATGTAACCCACTCGTGCACCCAACTGATC |
| | TTCAGCATCTTTTACTTTCACCAGCGTTTCTGGGTGAGCAAAAACAGGAAGGCAAAATGCCGCAAAAAA |
| | GGGAATAAGGGCGACACGGAAATGTTGAATACTCATACTCTTCCTTTTTCAATATTATTGAAGCATTTAT |
| | CAGGGTTATTGTCTCATGAGCGGATACATATTTGAATGTATTTAGAAAAATAAACAAATAGGGGTTCCG |
| | CGCACATTTCCCCGAAAAGTGCCACCTGGGTCGACATTGATTATTGA |
| Note | CMV enhancer |
| | CMV promoter |
| | Kozak sequence |
| | eGFP orf (Human codon optimized) |
| | β-globin PolyA signal |
| | G132 primer |
| | G133 primer |

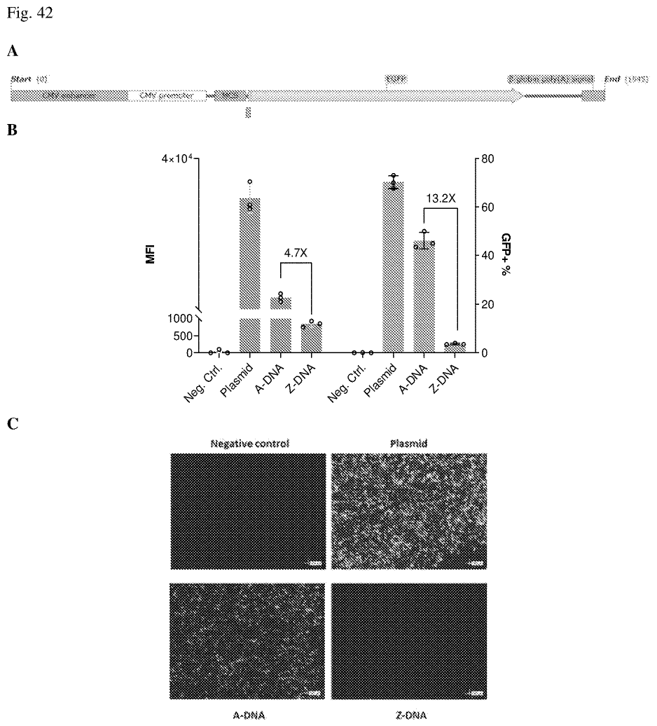

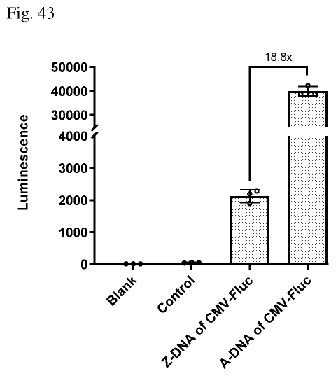

An average of 3.64% GFP+ cells were detected in HEK293T cells transfected with the Z-DNA cassette for eGFP, which was 12.2-fold lower than transfection with A-DNA (FIG. 26G and FIG. 42). To assess whether mammalian cells could decode longer Z-DNA cargo, we constructed another cassette containing a firefly luciferase (Fluc) gene with a total length of 2.5 kb. Luciferase activity was detected in HEK293T cells transfected with Z-DNA. However, the luciferase activity was reduced by 18.8-fold compared to A-DNA (FIG. 43). Next, we tested Z-DNA in HeLa human cervical cancer cell lines. Interestingly, HeLa cells showed a 2.6-fold increase in compatibility of eGFP compared to HEK293T cells (relative compatibility 20% for HeLa cells versus 7.6% for HEK293T cells, relative compatibility means the ratio of GFP+% with Z-DNA to A-DNA) (FIG. 25H and I). Together, these results indicate that transcription and translation systems function with Z-substituted DNA in both prokaryotic and eukaryotic organisms.

### Example 10: Human Cells Showed High Compatibility with mRNA Written by ZUGC

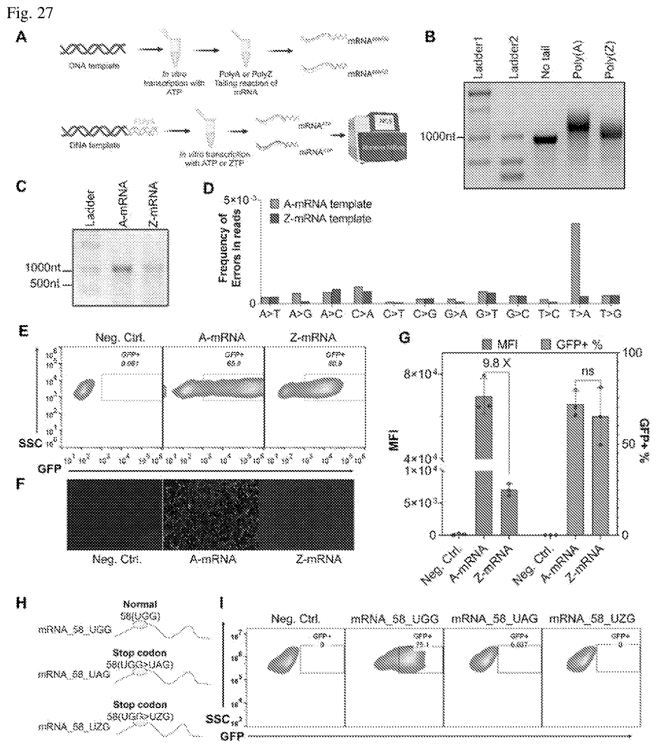

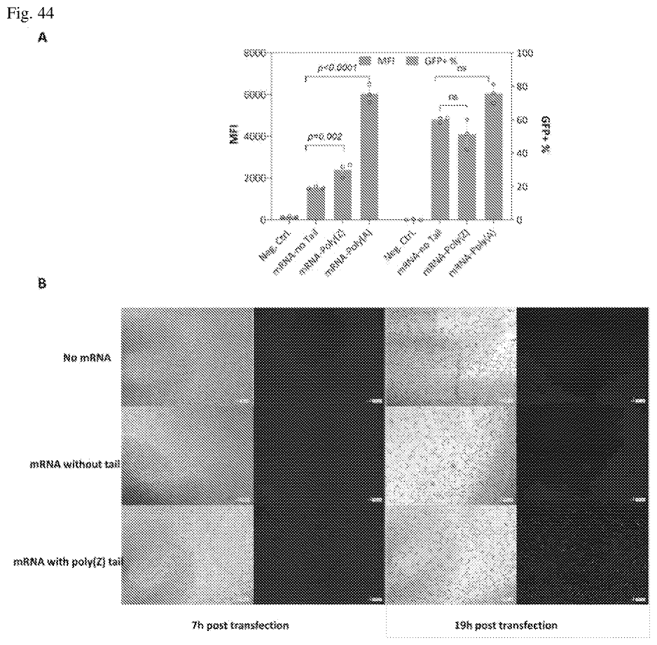

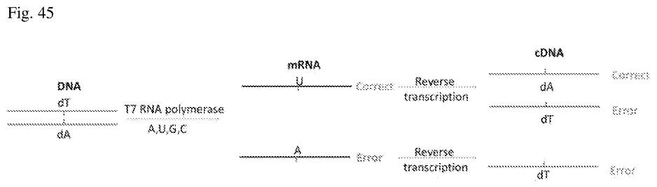

Decoding exogenous DNA in mammalian cells involves a complex process including DNA delivery to the cell's nucleus, transcription, and translation. Alternatively, mRNA can be directly translated after entering the cytoplasm to complete the expression of the target protein. Indeed, the delivery of standard mRNA shows faster and stronger protein expression than standard DNA (15). From this, we reasoned that mammalian cells may have greater compatibility with ZUGC-written mRNA. We investigated this by first using IVT reactions to produce mRNA both with and without Z substitutions (FIG. 27A). We then explored whether ZTP could replace adenosine triphosphate (ATP) and execute biological functions in the form of a poly(Z) tail. In eukaryotes, poly(A) tails play a key role in enhancing mRNA stability and expression (33). Poly(A) polymerase from E. coli succeeded in tailing ZTP to normal mRNA of eGFP 875 nucleotides (nt) long to generate mRNA-poly(Z) products (FIG. 27B). This result implies that while ZTP-based tailing is achievable, the process is notably less efficient than tailing with ATP. 24 hours after transfection, mRNA-poly(Z) showed 50% more GFP expression than mRNA with no tails (FIG. 44). Then, ZUGC-mRNA transcripts were produced from a DNA template (Table 12) carrying a GG start site of transcription using a T7 RNA polymerase reaction using either only ATP or ZTP (FIG. 27C). Negative impacts of Z-substitution on the accuracy of IVT were not observed from NGS analysis (FIG. 27D). Among these, the T>A mutations were reduced by 10-fold compared to the standard template. We speculated that the 2-amino substituent in adenosine led lower Z-A mismatch than A-A during the transcription process (FIG. 45) (34). To test whether non-canonical mRNA could be translated in mammalian cells, we delivered Z-mRNA of eGFP to HEK293T cells in vitro. We observed that the cells transfected with Z-mRNA showed detectable GFP expression (FIG. 27E and F). Notably, there was no statistical difference in the GFP positive fraction between Z-mRNA and A-mRNA transfected HEK293T cells. However, standard mRNA produced an MFI that was 9.13-fold higher than the Z-substituted mRNA. Interestingly, the Z-mRNA resulted in an average of 65.2% GFP+ cells, which was 19-fold higher than Z-DNA transfection (FIG. 27G). These data demonstrate that HEK293T cells have high compatibility with Z-mRNA encoding eGFP. Next, we explored whether Z-mRNA affects the preciseness of protein translation in mammalian cells. To do this, we mutated the 58_UGG codon encoding tryptophan (W) at position 58 of the reporter DNA template to 58_UAG, a stop codon. Then 58_UZG Z-mRNA was produced from this variant template (FIG. 27H). No GFP+ cells were detected in HEK293T cells transfected with 58_UZG-mRNA (FIG. 27/). This demonstrates that the UZG codon was properly read as a stop codon and not tryptophan in HEK293T cells. This is also consistent with high fidelity in vitro amplification and transcription. These results demonstrate that the ZUGC codon system can be compatible and function in mammalian cells, and that HEK293T cells have higher compatibility with Z-RNA than Z-DNA in eGFP expression.

### Example 11: ZUGC-crRNA Enhances In Vitro Cleavage Activity of Cas12a

The high compatibility of ZUGC-mRNA in mammalian cells encouraged us to explore more Z-RNA-related applications. Gene editing with RNA-guided endonucleases has been widely used to advance fundamental research and for applications in animals, plants, and humans (35). We speculated that nucleases may have high compatibility with ZUGC-guide-RNA as they often rely on a small sizes RNA to function. LbCas12a from Lachnospiraceae bacterium is a Type V CRISPR associated protein (Cas) effector that prefers a T-rich 5′-TTTN Protospacer Adjacent Motif (PAM) (36, 37). The single guide RNA (gRNA) used for LbCas12a consists only of a 39-nt CRISPR RNA (crRNA) with a single stem loop, making it notably simpler (FIG. 28A). We first tested the cleavage activity of Cas12a programed with ZUGC-crRNA (Z-crRNA) on plasmid DNA substrates. We selected a 20-nt guide sequence containing two A-bases to target a DNA site bearing a 5′-TTTA PAM (FIG. 28A and Table 12). The corresponding crRNA was 39-nt in length, where A occupied a total of seven positions. We observed that Z-crRNA was able to efficiently induce Cas12a-mediated cleavage of supercoiled DNA (FIG. 28B). Notably, plasmid cleavage activity was improved up to 3.3-fold when using Z-crRNA compared to A-crRNA (FIG. 28C). We also mapped the Z-crRNA cleavage site using Sanger sequencing of the cleaved DNA ends and found that the cleavage site was same as when using A-crRNA (FIG. 28D).

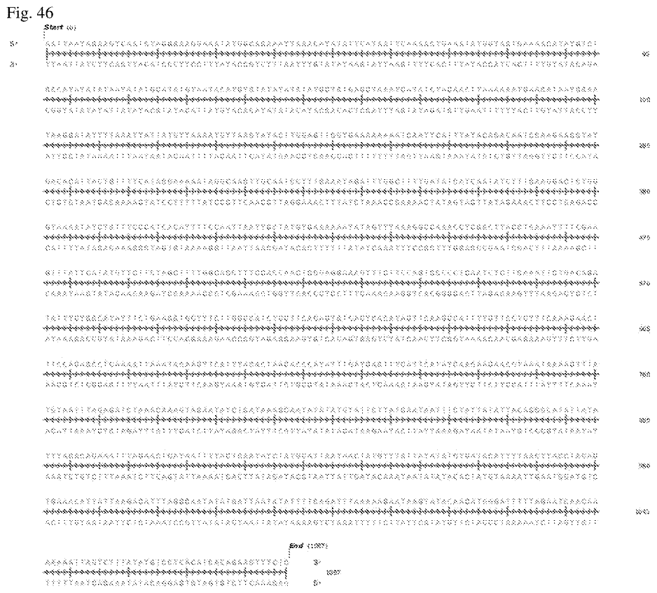

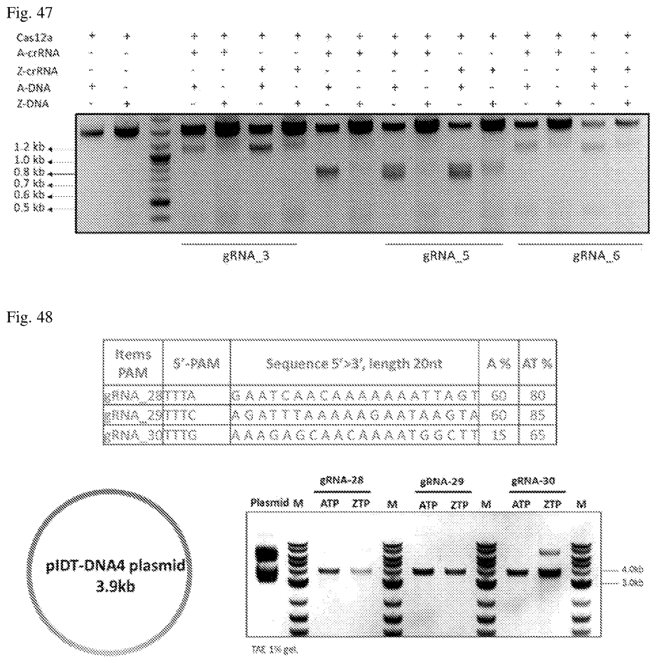

To investigate whether Cas12a could mediate cleavage of Z-DNA, PCR products with a ZT-content of 63.5% were amplified from a plasmid carrying an artificial sequence, DNA4 (FIG. 46 and Table 15), and were used as substrates. Three Cas12a crRNAs containing a 20-nt guide sequence complimentary to target DNA sites with an A/Z content between 15% to 35% and AT/ZT content 45-75% were designed (FIG. 28E and F). Both Z-crRNA and A-crRNA were able to guide Cas12a to the target and cleave PCR products with and without Z-substitutions. Notably, Z-crRNA showed 1.4 to 1.8-fold higher cleavage activity than A-crRNA on standard DNA, and up to 6-fold more activity than A-crRNA on Z-DNA substrates (FIG. 28G and FIG. 47) and showed activity on both 5′-TTTC and 5′-TTTG PAMs as well (FIG. 48). Previous studies reported that crRNA with chemical modifications on terminal nucleotides could enhance serum stability (38, 39). The Z-substitution did not improve the resistance of crRNA to fetal bovine serum (FIG. 49).

TABLE 15
Plasmid carrying DNA4 sequence.

| Name | pIDT-DNA4 |
|---|---|
| Sequence | TCGCGCGTTTCGGTGATGACGGTGAAAACCTCTGACACATGCAGCTCCCGGAGACGGTCACAGCTTGTC |
| | TGTAAGCGGATGCCGGGAGCAGACAAGCCCGTCAGGGCGCGTCAGCGGGTGTTGGCGGGTGTCGGGG |
| | CTGGCTTAACTATGCGGCATCAGAGCAGATTGTACTGAGAGTGCACCAAATGCGGTGTGAAATACCGCA |
| | CAGATGCGTAAGGAGAAAATACCGCATCAGGCGCCATTCGCCATTCAGGCTGCGCAACTGTTGGGAAG |
| | GGCGATCGGTGCGGGCCTCATCGCTATTACGCCAGCTGGCGAAAGGGGGATGTGCTGCAAGGCGATTA |
| | AGTTGGGTAACGCCAGGGTTTTCCCAGTCACGACGTTGTAAAACGACGGCCAGTGCAACGCGATGACG |
| | ATGGATAGCGATTCATCGATGAGCTGACCCGATCGCCGCCGCCGGAGGGTTGCGTTTGAGACGGGCGA |
| | CAGATAATTAATAGAAGTCAATGTAGGGAAGGAAATATGGCAGAAATTAAACATATATTCATAATTC |
| | AAAAGTGAAATATGGTAGTGAAAACATATGTCTGCCATATATATAATATATGCATATGTAATACATGT |
| | GTATATATGTATGCTGTGAGCTAAATCATATCTACAACTTAAAAAATGAACATAATGGAATAAGGATA |
| | TTTTAAATTATTATGTTAAAATGTTAAGTATACTTGGAGTTGGTGAAAAAAAATCAATTCATTTATACA |
| | GACAATCCAAGAAGGTATGACACATTACTGTTTTCATAGGAAAAATAGGCAAGTTGCAATCCTTTGAA |
| | ATAGATTTGGCTTTTGATATCATCAATATCTTTGAAGGACTCTGGGTAAAATATCTGTTTCCCATCACAT |
| | TTTCCAATTAATTGCTATGTGAAAAAATATAGTTTAAAGGCCAAACCTCGGCTTACCTGAAATTTTCGA |
| | AGTTTATTCATATGTTCTTCTAGCTTTTGGCAGCTTTCCACCAACTGGGAGGAAAGTTTCTTCCAGTGCC |
| | CCTCAATCTCTTCAAATTCTGACAGATATTTCTGGCATATTTCTGAAGGTGCTTTCTTGGCCATCTCCTTC |
| | ACAGTGTCACTCAGATAGTTGAAGCCATTTTGTTGCTCTTTCAAAGAACTTTGCAGAGCCTCAAAATTA |
| | AATAGAAGTTCATTTACACTAACACGCATATTTGATGAGTTTCATTCATATCAAGAAGAAGGTAAATA |
| | AAAGTTTATGTAATTTAGAGATCTAAACAAAGTAGAATATCTGATAAAGCAATATATATGTATTCTTAT |
| | GAATAATTTCTATTATATTACAGGGCATATTATATTTAGACAGAAATTTAGAAGTCATAATTTTACTGA |
| | ATATCTATGCATTAATAACTATGTTTATTATATATGTGATACATTTTAACTTACCTACAGTGAAACATTA |
| | TTAAGACATTTAGGCAATATATGATTAATATATTTTCAGATTTAAAAAGAATAAGTATACAACATGGG |
| | ATTTTTAGAATCAACAAAAAAATTAGTCTTTATATGTCCTCACATCACAGAAGTTTCTCATCAGTTCTGG |
| | ACCAGCGAGCTGTGCTGCGACTCGTGGCGTAATCATGGTCATAGCTGTTTCCTGTGTGAAATTGTTATCC |
| | GCTCACAATTCCACACAACATACGAGCCGGAAGCATAAAGTGTAAAGCCTGGGGTGCCTAATGAGTGA |
| | GCTAACTCACATTAATTGCGTTGCGCTCACTGCCCGCTTTCCAGTCGGGAAACCTGTCGTGCCAGCTGCA |
| | TTAATGAATCGGCCAACGCGCGGGGAGAGGCGGTTTGCGTATTGGGCGCTCTTCCGCTTCCTCGCTCAC |
| | TGACTCGCTGCGCTCGGTCGTTCGGCTGCGGCGAGCGGTATCAGCTCACTCAAAGGCGGTAATACGGTT |
| | ATCCACAGAATCAGGGGATAACGCAGGAAAGAACATGTGAGCAAAAGGCCAGCAAAAGGCCAGGAAC |
| | CGTAAAAAGGCCGCGTTGCTGGCGTTTTTCCATAGGCTCCGCCCCCCTGACGAGCATCACAAAAATCGA |
| | CGCTCAAGTCAGAGGTGGCGAAACCCGACAGGACTATAAAGATACCAGGCGTTTCCCCCTGGAAGCTCC |
| | CTCGTGCGCTCTCCTGTTCCGACCCTGTCGCTTACCGGATACCTGTCCGCCTTTCTCCCTTCGGGAAGCGT |
| | GGCGCTTTCTCATAGCTCACGCTGTAGGTATCTCAGTTCGGTGTAGGTCGTTCGCTCCAAGCTGGGCTGT |
| | GTGCACGAACCCCCCGTTCAGCCCGACCGCTGCGCCTTATCCGGTAACTATCGTCTTGAGTCCAACCCGG |
| | TAAGACACGACTTATCGCCACTGGCAGCAGCCACTGGTAACAGGATTAGCAGAGCGAGGTATGTAGGC |
| | GGTGCTACAGAGTTCTTGAAGTGGTGGCCTAACTACGGCTACACTAGAAGAACAGTATTTGGTATCTGC |
| | GCTCTGCTGAAGCCAGTTACCTTCGGAAAAAGAGTTGGTAGCTCTTGATCCGGCAAACAAACCACCGCT |
| | GGTAGCGGTGGTTTTTTTGTTTGCAAGCAGCAGATTACGCGCAGAAAAAAAGGATCTCAAGAAGATCCT |
| | TTGATCTTTTCTACGGGGTCTGACGCTCAGTGGAACGAAAACTCACGTTAAGGGATTTTGGTCATGAGAT |
| | TATCAAAAAGGATCTTCACCTAGATCCTTTTAAATTAAAAATGAAGTTTTAAATCAATCTAAAGTATATAT |
| | GAGTAAACTTGGTCTGACAGTTACCAATGCTTAATCAGTGAGGCACCTATCTCAGCGATCTGTCTATTTC |
| | GTTCATCCATAGTTGCCTGACTCCCCGTCGTGTAGATAACTACGATACGGGAGGGCTTACCATCTGGCCC |
| | CAGTGCTGCAATGATACCGCGAGACCCACGCTCACCGGCTCCAGATTTATCAGCAATAAACCAGCCAGC |
| | CGGAAGGGCCGAGCGCAGAAGTGGTCCTGCAACTTTATCCGCCTCCATCCAGTCTATTAATTGTTGCCG |
| | GGAAGCTAGAGTAAGTAGTTCGCCAGTTAATAGTTTGCGCAACGTTGTTGCCATTGCTACAGGCATCGT |
| | GGTGTCACGCTCGTCGTTTGGTATGGCTTCATTCAGCTCCGGTTCCCAACGATCAAGGCGAGTTACATGA |
| | TCCCCCATGTTGTGCAAAAAAGCGGTTAGCTCCTTCGGTCCTCCGATCGTTGTCAGAAGTAAGTTGGCCG |
| | CAGTGTTATCACTCATGGTTATGGCAGCACTGCATAATTCTCTTACTGTCATGCCATCCGTAAGATGCTTT |
| | TCTGTGACTGGTGAGTACTCAACCAAGTCATTCTGAGAATAGTGTATGCGGCGACCGAGTTGCTCTTGCC |
| | CGGCGTCAATACGGGATAATACCGCGCCACATAGCAGAACTTTAAAAGTGCTCATCATTGGAAAACGTT |
| | CTTCGGGGCGAAAACTCTCAAGGATCTTACCGCTGTTGAGATCCAGTTCGATGTAACCCACTCGTGCACC |
| | CAACTGATCTTCAGCATCTTTTACTTTCACCAGCGTTTCTGGGTGAGCAAAAACAGGAAGGCAAAATGCC |
| | GCAAAAAAGGGAATAAGGGCGACACGGAAATGTTGAATACTCATACTCTACCTTTTTCAATATTATTGA |
| | AGCATTTATCAGGGTTATTGTCTCATGAGCGGATACATATTTGAATGTATTTAGAAAAATAAACAAATAG |
| | GGGTTCCGCGCACATTTCCCCGAAAAGTGCCACCTGACGTCTAAGAAACCATTATTATCATGACATTAAC |
| | CTATAAAAATAGGCGTATCACGAGGCCCTTTCGTC |
| Note | Restriction site SspI endonuclease |
| | G030 primer |
| | G031 primer |
| | Partial gene sequence from Mouse |

The first 5 nucleotides at the 5′-end of the guide sequence which are complimentary to the target DNA is known as the seed region for Cas12a (37, 40). As shown in FIGS. 28F and 28H, Cas12a efficiently mediated DNA cleavage with gRNAs carrying Z-substitutions in the seed region. Five A-bases are involved in the formation of the pseudoknot structure required for active Cas12a in the 5′-handle of standard crRNA where G(-6)-A(-2) and U(-15)-C(-11) form a stem structure via five Watson-Crick base pairs [G(-6):C (-11)-A(-2):U(-15)] (FIG. 28A) (36, 37). The ability of ZUGC-crRNA to function properly in Cas12a cleavage suggests that Z-base substitutions can also lead to the formation of the functional structure via non-Watson-Crick base pairing. Thus, our data demonstrates that Cas12a has a good tolerance for this non-canonical base pairing structure.

In the standard targeting processes, Cas12a-crRNA complexes with its complimentary DNA target to form a DNA-RNA heteroduplex via 20 Watson-Crick base pairs prior to DNA cleavage. This heteroduplex is stabilized by H-bonds between base pairs (41, 42). Because Z-bases allow for a third hydrogen bond between Z and T, we hypothesized that more H-bonds between the DNA and crRNA may benefit the stability of the heteroduplex and lead to increased endonuclease cleavage. To evaluate this, the pIDT-DNA4 plasmid (Table 15) linearized by SspI was selected for further investigation. Eight gRNAs with AT-contents ranging from 45% to 85% that target 20-nt DNA sites bearing a 5′-TTTA PAM were selected (FIG. 28H). Interestingly, time curves of a cleavage assay revealed that Z-crRNA showed 12%-41% greater cleavage activity than A-crRNA across all tested crRNAs (FIG. 28/). Then we conducted sequence logo analysis for the eight guide sequences to understand the sequence features. As shown in FIG. 28J, when T-bases appeared at position 5′-PAM-6, 8, 10, 18, 19-3′, Z-crRNA showed higher activity than A-crRNA. These results can be adapted for the optimal design of Z-crRNA to enhance the cleavage activity of Cas12a on another DNA sequence (FIG. 50 and Table 16). To our knowledge, no research shows that chemically modified RNA bases could improve target affinity of crRNA-DNA to enhance CRISPR effector cleavage activity. As shown in FIG. 25D, when the DNA length is less than 20 bp, the replacement of A with Z bases increases the melting temperature. We reasoned that improved Cas12a cleavage efficiencies are due to improved binding affinity of crRNA:DNA. Previous studies reported that crRNA or sgRNA with chemical modifications show similar or lower in vitro cleavage activity compared to unmodified guides (43, 44). Here we demonstrate the non-canonical base that could enhance Cas12a in vitro cleavage activity.

TABLE 16
Plasmid carrying EMX1 sequence.

| Name | pIDT-EMX1 |
|---|---|
| Sequence | TCGCGCGTTTCGGTGATGACGGTGAAAACCTCTGACACATGCAGCTCCCGGAGACGGTCACAGCTTGTC |
| | TGTAAGCGGATGCCGGGAGCAGACAAGCCCGTCAGGGCGCGTCAGCGGGTGTTGGCGGGTGTCGGGG |
| | CTGGCTTAACTATGCGGCATCAGAGCAGATTGTACTGAGAGTGCACCAAATGCGGTGTGAAATACCGCA |
| | CAGATGCGTAAGGAGAAAATACCGCATCAGGCGCCATTCGCCATTCAGGCTGCGCAACTGTTGGGAAG |
| | GGCGATCGGTGCGGGCCTCATCGCTATTACGCCAGCTGGCGAAAGGGGGATGTGCTGCAAGGCGATTA |
| | AGTTGGGTAACGCCAGGGTTTTCCCAGTCACGACGTTGTAAAACGACGGCCAGTGCAACGCGATGACG |
| | ATGGATAGCGATTCATCGATGAGCTGACCCGATCGCCGCCGCCGGAGGGTTGCGTTTGAGACGGGCGA |
| | CAGATCCATCCCCTTCTGTGAATGTTAGACCCATGGGAGCAGCTGGTCAGAGGGGACCCCGGCCTGGG |
| | GCCCCTAACCCTATGTAGCCTCAGTCTTCCCATCAGGCTCTCAGCTCAGCCTGAGTGTTGAGGCCCCAG |
| | TGGCTGCTCTGGGGGCCTCCTGAGTTTCTCATCTGTGCCCCTCCCTCCCTGGCCCAGGTGAAGGTGTGG |
| | TTCCAGAACCGGAGGACAAAGTACAAACGGCAGAAGCTGGAGGAGGAAGGGCCTGAGTCCGAGCA |
| | GAAGAAGAAGGGCTCCCATCACATCAACCGGTGGCGCATTGCCACGAAGCAGGCCAATGGGGAGGA |
| | CATCGATGTCACCTCCAATGACTAGGGTGGGCAACCACAAACCCACGAGGGCAGAGTGCTGCTTGCTG |
| | CTGGCCAGGCCCCTGCGTGGGCCCAAGCTGGACTCTGGCCACTCCCTGGCCAGGCTTTGGGGAGGCCT |
| | GGAGTCATGGCCCCACAGGGCTTGAAGCCCGGGGCCGCCATTGACAGAGGGACAAGCAATGGGCTG |
| | GCTGAGGCCTGGGACCACTTGGCCTTCTCCTCGGAGAGCCTGCCTGCCTGGGCGGGCCCGCCCGCCAC |
| | CGCAGCCTCCCAGCTGCTCTCCGTGTCTCCAATCTCCATCAGTTCTGGACCAGCGAGCTGTGCTGCGACT |
| | CGTGGCGTAATCATGGTCATAGCTGTTTCCTGTGTGAAATTGTTATCCGCTCACAATTCCACACAACATA |
| | CGAGCCGGAAGCATAAAGTGTAAAGCCTGGGGTGCCTAATGAGTGAGCTAACTCACATTAATTGCGTTG |
| | CGCTCACTGCCCGCTTTCCAGTCGGGAAACCTGTCGTGCCAGCTGCATTAATGAATCGGCCAACGCGCG |
| | GGGAGAGGCGGTTTGCGTATTGGGCGCTCTTCCGCTTCCTCGCTCACTGACTCGCTGCGCTCGGTCGTTC |
| | GGCTGCGGCGAGCGGTATCAGCTCACTCAAAGGCGGTAATACGGTTATCCACAGAATCAGGGGATAAC |
| | GCAGGAAAGAACATGTGAGCAAAAGGCCAGCAAAAGGCCAGGAACCGTAAAAAGGCCGCGTTGCTGG |
| | CGTTTTTCCATAGGCTCCGCCCCCCTGACGAGCATCACAAAAATCGACGCTCAAGTCAGAGGTGGCGAA |
| | ACCCGACAGGACTATAAAGATACCAGGCGTTTCCCCCTGGAAGCTCCCTCGTGCGCTCTCCTGTTCCGAC |
| | CCTGTCGCTTACCGGATACCTGTCCGCCTTTCTCCCTTCGGGAAGCGTGGCGCTTTCTCATAGCTCACGCT |
| | GTAGGTATCTCAGTTCGGTGTAGGTCGTTCGCTCCAAGCTGGGCTGTGTGCACGAACCCCCCGTTCAGC |
| | CCGACCGCTGCGCCTTATCCGGTAACTATCGTCTTGAGTCCAACCCGGTAAGACACGACTTATCGCCACT |
| | GGCAGCAGCCACTGGTAACAGGATTAGCAGAGCGAGGTATGTAGGCGGTGCTACAGAGTTCTTGAAGT |
| | GGTGGCCTAACTACGGCTACACTAGAAGAACAGTATTTGGTATCTGCGCTCTGCTGAAGCCAGTTACCTT |
| | CGGAAAAAGAGTTGGTAGCTCTTGATCCGGCAAACAAACCACCGCTGGTAGCGGTGGTTTTTTTGTTTG |
| | CAAGCAGCAGATTACGCGCAGAAAAAAAGGATCTCAAGAAGATCCTTTGATCTTTTCTACGGGGTCTGA |
| | CGCTCAGTGGAACGAAAACTCACGTTAAGGGATTTTGGTCATGAGATTATCAAAAAGGATCTTCACCTA |
| | GATCCTTTTAAATTAAAAATGAAGTTTTAAATCAATCTAAAGTATATATGAGTAAACTTGGTCTGACAGTT |
| | ACCAATGCTTAATCAGTGAGGCACCTATCTCAGCGATCTGTCTATTTCGTTCATCCATAGTTGCCTGACTC |
| | CCCGTCGTGTAGATAACTACGATACGGGAGGGCTTACCATCTGGCCCCAGTGCTGCAATGATACCGCGA |
| | GACCCACGCTCACCGGCTCCAGATTTATCAGCAATAAACCAGCCAGCCGGAAGGGCCGAGCGCAGAAG |
| | TGGTCCTGCAACTTTATCCGCCTCCATCCAGTCTATTAATTGTTGCCGGGAAGCTAGAGTAAGTAGTTCG |
| | CCAGTTAATAGTTTGCGCAACGTTGTTGCCATTGCTACAGGCATCGTGGTGTCACGCTCGTCGTTTGGTA |
| | TGGCTTCATTCAGCTCCGGTTCCCAACGATCAAGGCGAGTTACATGATCCCCCATGTTGTGCAAAAAAGC |
| | GGTTAGCTCCTTCGGTCCTCCGATCGTTGTCAGAAGTAAGTTGGCCGCAGTGTTATCACTCATGGTTATG |
| | GCAGCACTGCATAATTCTCTTACTGTCATGCCATCCGTAAGATGCTTTTCTGTGACTGGTGAGTACTCAAC |
| | CAAGTCATTCTGAGAATAGTGTATGCGGCGACCGAGTTGCTCTTGCCCGGCGTCAATACGGGATAATAC |
| | CGCGCCACATAGCAGAACTTTAAAAGTGCTCATCATTGGAAAACGTTCTTCGGGGCGAAAACTCTCAAG |
| | GATCTTACCGCTGTTGAGATCCAGTTCGATGTAACCCACTCGTGCACCCAACTGATCTTCAGCATCTTTTA |
| | CTTTCACCAGCGTTTCTGGGTGAGCAAAAACAGGAAGGCAAAATGCCGCAAAAAAGGGAATAAGGGCG |
| | ACACGGAAATGTTGAATACTCATACTCTACCTTTTTCAATATTATTGAAGCATTTATCAGGGTTATTGTCT |
| | CATGAGCGGATACATATTTGAATGTATTTAGAAAAATAAACAAATAGGGGTTCCGCGCACATTTCCCCG |
| | AAAAGTGCCACCTGACGTCTAAGAAACCATTATTATCATGACATTAACCTATAAAAATAGGCGTATCACG |
| | AGGCCCTTTCGTC |
| Note | Restriction site of SspI |
| | G030 primer |
| | G031 primer |
| | Partial EMX1 gene sequence from Human genome |

Together, these results show that Cas 12a has good compatibility with both Z-DNA and Z-crRNA. This means that Z-crRNA could act as a potentially new strategy for in vitro screening highly active guide RNAs for gene manipulation in somatic cells with LNP delivery (45).

### Example 12: Z-crRNA:TracrRNA Duplex Enables Efficient Cas9-Catalyzed Non-Watson-Crick DNA Cleavage In Vitro

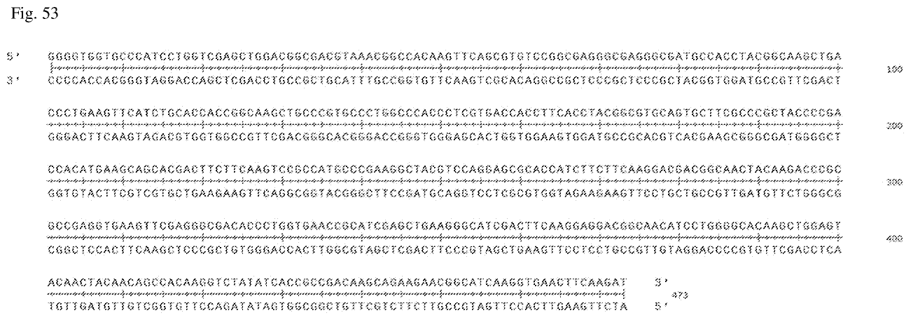

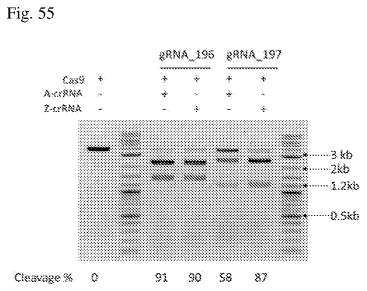

As a Type II CRISPR/Cas system, SpCas9 is the most widely used tool in the field of gene editing therapies (35, 46). In contrast to Cas12a, the G-rich PAM 5′-NGG is favored by Cas9. Compared to the 39-nt crRNA for Cas12a, SpCas9 guide RNA is comprised of both a 42-nt crRNA and an 80-nt trans-activating crRNA (tracrRNA) (FIG. 29A). SpCas9 can also be programmed by a ~90-nt single guide RNA (sgRNA) to cleave a target sequence (FIG. 29B) (47). Overall, the gRNAs for SpCas9 are longer and more complex than LbCas12a. This led us to investigate whether Z-crRNA or Z-sgRNA could mediate SpCas9 cleavage of a DNA substrate. We first performed cleavage tests on supercoiled plasmid substrates with different dual-RNAs. Z-crRNA:Z-tracrRNA impeded Cas9 cleavage activity reducing it by ~50%, whereas Z-crRNA:A-tracrRNA led to similar Cas9 cleavage activity as the standard crRNA:tracrRNA (FIG. 29C). Though ZUGC-sgRNA produced by IVT showed the same quality as A-sgRNA, cleavage functionality was not observed for Cas9 loaded with Z-sgRNA (FIG. 5I). The Cas9-Z-sgRNA complexes also showed no detectable activity on other sites bearing a 5′-AGG PAM (FIG. 52A and B). A previous study showed that Z-sgRNA could strongly block the cleavage activity of SpCas9 on standard PCR products (14). Our results demonstrated that when employing dual gRNAs (crRNA:tracrRNA) rather than a single guide targeted to A-rich (~35-60%) regions, SpCas9 activity can be preserved when Z-crRNA is utilized, with a 50% retention rate when using Z-tracrRNA and a 100% retention rate when using A-tracrRNA. We speculated that pairing of the non-Watson-Crick form of Z-U failed in the formation of the active secondary structure of tracrRNA and sgRNA required for Cas9 (48). Next, we investigated the effect of Z-substituted guide RNAs on Cas9-mediated cleavage of Z-DNA or A-DNA PCR products. Our results indicated that Z-crRNA showed the same activity as standard crRNA when acting on Z-DNA or A-DNA. Like in the plasmid cleavage assay experiments, Z-tracrRNA led to detectable cleavage of PCR products with ~50% lower activity than A-tracRNA (FIG. 29D and E). However, Z-sgRNAs still failed to cleave Z-DNA or A-DNA PCR products of the DNA5 template (FIG. 52C-E and FIG. 53). In vitro cleavage activities on AT-rich or ZT-rich DNA were also evaluated (FIG. 29F). We observed that Z-crRNA boosted cleavage activity on PCR products up to 60% more compared to A-crRNA (FIG. 29G and FIG. 54). This similar improvement was also observed in the cleavage assay of linear plasmid DNA (FIG. 55). These results indicate that SpCas9 is compatible with both Z-DNA and Z-RNA. Overall, SpCas9 shows higher relative activities than REs and Cas12a on Z-DNA (FIG. 29H and I). We reasoned that the CRISPR-Cas9 system should be an efficient mechanism in nature to defend against phage or virus carrying the Z-DNA genome.

TABLE 17
Plasmid carrying DNA6 sequence.

| Name | pIDT-DNA6 |
|---|---|
| Sequence | CCCGTGTAAAACGACGGCCAGTTTATCTAGTCAGCTTGATTCTAGCTGATCGTGGACCGGAAGGTGAGC |
| | CAGTGAGTTGATTGCAGTCCAGTTACGCTGGAGTCTGAGGCTCGTCCTGAATGATATGCGGCCTCGTCA |
| | AGAAAGCAGAGACTGCCGGGAGCAAAGAGAATAGACTGTAGGGAATACTAAGCATAGACTCCAGGGT |
| | AAACAAAGCATAGACTGAGGGGAAGACCAAGGATAGACTGCTGGGAACATAAAGAATAGAATGATGG |
| | GACAAAGAGGAAGAGAGACGGGGACACACACACTTAGAATCTGTGGGAGTCTAAGCAGAAGAAGAAG |
| | AGGAACACAATGCATAGATTGCCGGGCGCGTGATCTTACGGCATTATACGTATGATCGGTCCACGATCA |
| | GCTAGATTATCTAGTCAGCTTGATGTCATAGCTGTTTCCTGAGGCTCAATACTGACCATTTAAATCATACC |
| | TGACCTCCATAGCAGAAAGTCAAAAGCCTCCGACCGGAGGCTTTTGACTTGATCGGCACGTAAGAGGTT |
| | CCAACTTTCACCATAATGAAATAAGATCACTACCGGGCGTATTTTTTGAGTTATCGAGATTTTCAGGAGC |
| | TAAGGAAGCTAAAATGAGTATTCAACATTTCCGTGTCGCCCTTATTCCCTTTTTTGCGGCATTTTGCCTTC |
| | CTGTTTTTGCTCACCCAGAAACGCTGGTGAAAGTAAAAGATGCTGAAGATCAGTTGGGTGCACGAGTGG |
| | GTTACATCGAACTGGATCTCAACAGCGGTAAGATCCTTGAGAGTTTACGCCCCGAAGAACGTTTTCCAAT |
| | GATGAGCACTTTTAAAGTTCTGCTATGTGGCGCGGTATTATCCCGTATTGACGCCGGGCAAGAGCAACT |
| | CGGTCGCCGCATACACTATTCTCAGAATGACTTGGTTGAGTACTCACCAGTCACAGAAAAGCATCTCACG |
| | GATGGCATGACAGTAAGAGAATTATGCAGTGCTGCCATAACCATGAGTGATAACACTGCGGCCAACTTA |
| | CTTCTGGCAACGATCGGAGGACCGAAGGAGCTAACCGCTTTTTTGCACAACATGGGGGATCATGTAACT |
| | CGCCTTGATCGTTGGGAACCGGAGCTGAATGAAGCCATACCAAACGACGAGCGTGACACCACGATGCC |
| | TGTAGCAATGGCAACAACGTTGCGCAAACTATTAACTGGCGAACTACTTACTCTAGCTTCCCGGCAACAA |
| | TTAATAGACTGGATGGAGGCGGATAAAGTTGCAGGATCACTTCTGCGCTCGGCCCTCCCGGCTGGCTGG |
| | TTTATTGCTGATAAATCTGGAGCCGGTGAGCGTGGGTCTCGCGGTATCATTGCAGCACTGGGGCCAGAT |
| | GGTAAGCCCTCCCGCATCGTAGTTATCTACACGACGGGGAGTCAGGCAACTATGGATGAACGAAATAG |
| | ACAGATCGCTGAGATAGGTGCCTCACTGATTAAGCATTGGTAATGAGGGCCCAAATGTAATCACCTGGC |
| | TCACCTTCGGGTGGGCCTTTCTTGAGGACCTAAATGTAATCACCTGGCTCACCTTCGGGTGGGCCTTTCT |
| | GCGTTGCTGGCGTTTTTCCATAGGCTCCGCCCCCCTGACGAGCATCACAAAAATCGATGCTCAAGTCAGA |
| | GGTGGCGAAACCCGACAGGACTATAAAGATACCAGGCGTTTCCCCCTGGAAGCTCCCTCGTGCGCTCTC |
| | CTGTTCCGACCCTGCCGCTTACCGGATACCTGTCCGCCTTTCTCCCTTCGGGAAGCGTGGCGCTTTCTCAT |
| | AGCTCACGCTGTAGGTATCTCAGTTCGGTGTAGGTCGTTCGCTCCAAGCTGGGCTGTGTGCACGAACCC |
| | CCCGTTCAGCCCGACCGCTGCGCCTTATCCGGTAACTATCGTCTTGAGTCCAACCCGGTAAGACACGACT |
| | TATCGCCACTGGCAGCAGCCACTGGTAACAGGATTAGCAGAGCGAGGTATGTAGGCGGTGCTACAGAG |
| | TTCTTGAAGTGGTGGCCTAACTACGGCTACACTAGAAGAACAGTATTTGGTATCTGCGCTCTGCTGAAGC |
| | CAGTTACCTCGGAAAAAGAGTTGGTAGCTCTTGATCCGGCAAACAAACCACCGCTGGTAGCGGTGGTTT |
| | TTTTGTTTGCAAGCAGCAGATTACGCGCAGAAAAAAAGGATCTCAAGAAGATCCTTTGATTTTCTACCGA |
| | AGAAAGGCCCA |
| Note | G097 primer |
| | G098 primer |

### Example 13: Z-crRNA Guides Cas9 and Base Editor Facilitate Genome Editing in Human Cells

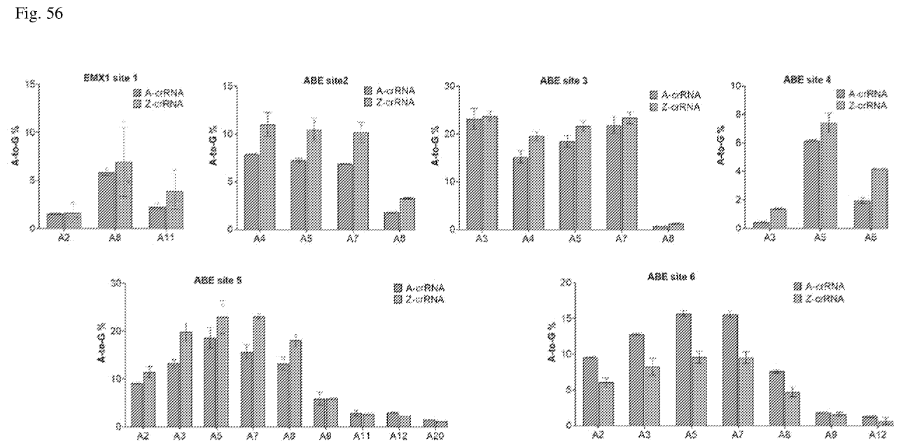

Considering SpCas9 is used much more widely than Cas12a, we further evaluated whether Z-crRNA could guide Cas9 to achieve specific gene editing in mammalian cells. As a proof of concept, we performed editing on endogenous genomic loci in human cells. First, we tried co-delivering SpCas9 mRNA and crRNA:tracRNA to HeLa cells. Compared to A-crRNA, Z-crRNA exhibited similar levels of insertion-deletion (indel) efficiency (2.38% versus 2.17% on average) (FIG. 30A). This is also consistent with the relative activity of in vitro cleavage (FIG. 29E). Analyzing indel patterns after editing, deletions did not exceed 7 bp in Z-crRNA editing products, while A-crRNA editing induced larger deletions up to 26 bp (FIG. 30B). As we know, adenine base editor (ABE) is a more powerful and safe tool used for gene editing (49, 50). We then tested whether Z-crRNA could mediate targeted A-to-G nucleotide conversions using an ABE8e. To do this, ABE8e mRNA and crRNA:tracrRNA were co-delivered to HeLa cells. Six endogenous genomic sites reported in previous studies (51) were employed to evaluate the A-to-G editing efficiency. As shown in FIG. 30C, A-crRNA and Z-crRNA induced A-to-G edits with average frequencies of 15.6% (6.19-28.71%) and 15.9% (7.48-32.44%), respectively. We did not observe dramatic differences of editing distribution in the editing windows between Z-crRNA and A-crRNA (FIG. 56). We then analyzed four potential off-target-sites to evaluate Cas9-dependent off-target editing activities. We found that Z-crRNA and A-crRNA induced off-target editing 0.15% on average (0.05-0.39%) and 0.39% (0.02-1.07%) at the four sites (FIG. 30D), respectively. Z-crRNA didn't induce more off-target editing than A-crRNA in our tested sites. These data demonstrate that Z-crRNA could guide an ABE editor to efficiently generate A-to-G conversions in mammalian cells.

Here we demonstrated that the biological function of ZTGC or ZUGC nucleic acids were explored at a different scale and for applications of protein expression and genome editing. Though Z-DNA shows changes in physical properties compared to A-DNA, it could be transcribed to mRNA and translated to functional proteins in standard prokaryotic and eukaryotic systems. Additionally, mammalian cells could also translate Z-mRNA into proteins. Z-mRNA showed greater eGFP expression efficiency than Z-DNA in HEK293T cells. Additionally, both type II CRISPR-Cas9 and type V CRISPR-Cas12a endonucleases can be guided by Z-crRNA to efficiently cleave targeted Z-DNA substrates. Z-substitutions did not change the attribute of PAM, which can be efficiently scanned and recognized by SpCas9 and LbCas12a. RNA-guided endonucleases can also work well with non-Watson-Crick base pairing processes. SpCas9 and base editors carried detectable editing efficiencies with Z-crRNA. Together, our data demonstrates the ability for multiple protein complexes to scan and recognize non-Watson-Crick DNA carrying Z-bases.

Various modified nucleobases and the enzymes responsible for their processing have been discovered in recent years (52-54). Scientists have been pursuing these discoveries to expand the genetic alphabet to have a diverse set of nucleobases for the creation of synthetic organisms (55-57). In the last few decades, more than 20 chemical modifications have been detected in eukaryotic mRNAs (58), but most of them do not alter Watson-Crick pairing. Modified nucleobases can also play critical roles in improving the efficacy of gene therapies and vaccines, including the FDA-approved SARS-CoV-2 mRNA vaccine (59-63). Though Z-substituted DNA worked less efficiently than standard DNA in these typical life systems for promoting transcription and protein expression, our research helps give new insights into understanding novel nucleosides and non-Watson-Crick DNA.

In order to expand the potential applications of gene editing, researchers have used protein engineering to alter the function of Cas nucleases, such as base or prime editors, or modified the PAM requirements of Cas9, such as in ultra-specific Cas9 and PAM free Cas9 (64-66). Scientists have also proposed various strategies for optimizing guide RNAs to modulate Cas9/Cas12a specificity and activity, including those with unknown mechanisms. Some of these strategies include incorporating 2′-deoxynucleotides (67), chemically modified ribonucleotides into guide RNAs (43, 68) and removing or extending nucleotides from or to the 5′ end of guide RNAs (69-72). Due to its PAM sequence (5′-TTTN PAM), Cas12a allows gene editing in regions of the human genome rich with AT sequences, such as untranslated regions (UTRs) or introns. 34% of genes are in AT-rich isochores, which represents 62% of the genome (73, 74). However, Cas12a's editing efficiency drastically decreased when the AT-content in the guide sequences increased (75, 76). For human genome editing, Cas9 guide sequences are most effective with a GC-content between 40 and 70%, and thus sgRNAs targeting 5′ and 3′ UTRs are highly ineffective (77, 78). Using Z-bases may be a potential strategy to improve activities of guide RNA through introducing non-Watson-Crick base pairing.

Our results show that Cas9 has similar or higher compatibility with non-Watson-Crick Z-DNA or Z-RNA, which enables Cas9 to be a potential research model used for exploring mechanism of interaction with Z-DNA or Z-RNA. Importantly, the ability for RNA-guided endonucleases to cleave Z-DNA could allow for a strategy to create a biosafety control approach for future synthetic viruses carrying a Z-DNA genome (79, 80).

### REFERENCES

1. M. D. Kirnos, I. Y. Khudyakov, N. I. Alexandrushkina, B. F. Vanyushin, 2-Aminoadenine is an adenine substituting for a base in S-2L cyanophage DNA. Nature 270, 369-370 (1977).
2. W. Grome Michael, J. Isaacs Farren, ZTCG: Viruses expand the genetic alphabet. Science 372, 460-461 (2021).
3. V. Pezo et al., Noncanonical DNA polymerization by aminoadenine-based siphoviruses. Science 372, 520-524 (2021).
4. D. Sleiman et al., A third purine biosynthetic pathway encoded by aminoadenine-based viral DNA genomes. Science 372, 516-520 (2021).
5. Y. Zhou et al., A widespread pathway for substitution of adenine by diaminopurine in phage genomes. Science 372, 512-516 (2021).
6. I. Y. Khudyakov, M. D. Kirnos, N. I. Alexandrushkina, B. F. Vanyushin, Cyanophage S-2L contains DNA with 2,6-diaminopurine substituted for adenine. Virology 88, 8-18 (1978).
7. D. Czernecki, F. Bonhomme, P.-A. Kaminski, M. Delarue, Characterization of a triad of genes in cyanophage S-2L sufficient to replace adenine by 2-aminoadenine in bacterial DNA. Nat. Commun. 12, 4710 (2021).
8. D. Czernecki, H. Hu, F. Romoli, M. Delarue, Structural dynamics and determinants of 2-aminoadenine specificity in DNA polymerase DpoZ of vibriophage $VC8. Nucleic Acids Res. 49, 11974-11985 (2021).
9. F. Lankaš et al., Critical Effect of the N2 Amino Group on Structure, Dynamics, and Elasticity of DNA Polypurine Tracts. Biophys. J. 82, 2592-2609 (2002).
10. Y. Lebedev et al., Oligonucleotides containing 2-aminoadenine and 5-methylcytosine are more effective as primers for PCR amplification than their nonmodified counterparts. Genetic Analysis: Biomolecular Engineering 13, 15-21 (1996).
11. M. Szekeres, A. V. Matveyev, Cleavage and sequence recognition of 2,6-diaminopurine-containing DNA by site-specific endonucleases. FEBS Letters 222, 89-94 (1987).
12. P. A. Kaminski, Mechanisms supporting aminoadenine-based viral DNA genomes. Cell. Mol. Life Sci. https://doi.org/10.1007/s00018-021-04055-7 (2021).
13. Y. Tan et al., Transcriptional Perturbations of 2,6-Diaminopurine and 2-Aminopurine. ACS Chem. Biol. 17, 1672-1676 (2022).
14. H. Yang et al., CRISPR-Cas9 recognition of enzymatically synthesized base-modified nucleic acids. Nucleic Acids Res. 51, 1501-1511 (2023).
15. K. Paunovska, D. Loughrey, J. E. Dahlman, Drug delivery systems for RNA therapeutics. Nat. Rev. Genet. 23, 265-280 (2022).
16. Q. Chen, Y. Zhang, H. Yin, Recent advances in chemical modifications of guide RNA, mRNA and donor template for CRISPR-mediated genome editing. Adv. Drug Deliv. Rev. 168, 246-258 (2021).
17. A. Mir et al., Heavily and fully modified RNAs guide efficient SpyCas9-mediated genome editing. Nat. Commun. 9, 2641 (2018).
18. N. Beiranvand, M. Freindorf, E. Kraka, Hydrogen Bonding in Natural and Unnatural Base Pairs-A Local Vibrational Mode Study. Molecules 26 (2021).
19. A. Hottin, A. Marx, Structural Insights into the Processing of Nucleobase-Modified Nucleotides by DNA Polymerases. Acc. Chem. Res. 49, 418-427 (2016).
20. M. Imashimizu, T. Oshima, L. Lubkowska, M. Kashlev, Direct assessment of transcription fidelity by high-resolution RNA sequencing. Nucleic Acids Res. 41, 9090-9104 (2013).
21. V. Potapov et al., Base modifications affecting RNA polymerase and reverse transcriptase fidelity. Nucleic Acids Res. 46, 5753-5763 (2018).
22. G. Weber et al., Thermal equivalence of DNA duplexes without calculation of melting temperature. Nature Physics 2, 55-59 (2006).
23. C. Bailly, M. J. Waring, The use of diaminopurine to investigate structural properties of nucleic acids and molecular recognition between ligands and DNA. Nucleic Acids Res. 26, 4309-4314 (1998).
24. M. Fernández-Sierra, Q. Shao, C. Fountain, L. Finzi, D. Dunlap, E. coli Gyrase Fails to Negatively Supercoil Diaminopurine-Substituted DNA. J. Mol. Biol. 427, 2305-2318 (2015).
25. C. Cheong, I. Tinoco, Jr., A. Chollet, Thermodynamic studies of base pairing involving 2,6-diaminopurine. Nucleic Acids Res. 16, 5115-5122 (1988).
26. J. Virstedt, T. Berge, R. M. Henderson, M. J. Waring, A. A. Travers, The influence of DNA stiffness upon nucleosome formation. J. Struct. Biol. 148, 66-85 (2004).
27. W. J. Chazin, M. Rance, A. Chollet, W. Luepin, Comparative NMR analysis of the decadeoxynucleotide d-(GCATTAATGC) 2 and an anlogue containing 2-aminoadenine. Nucleic Acids Res. 19, 5507-5513 (1991).
28. F. B. Howard, C.-w. Chen, J. S. Cohen, H. T. Miles, Poly(d2NH2A-dT): Effect of 2-amino substituent on the B to Z transition. Biochem. Biophys. Res. Commun. 118, 848-853 (1984).
29. A. Pingoud, A. Jeltsch, Structure and function of type II restriction endonucleases. Nucleic Acids Res. 29, 3705-3727 (2001).
30. F. Crick, Central Dogma of Molecular Biology. Nature 227, 561-563 (1970).
31. A. A. Komar, The Yin and Yang of codon usage. Hum. Mol. Genet. 25, R77-r85 (2016).
32. K. A. Curran et al., Short Synthetic Terminators for Improved Heterologous Gene Expression in Yeast. ACS Synth. Biol. 4, 824-832 (2015).
33. L. A. Passmore, J. Coller, Roles of mRNA poly(A) tails in regulation of eukaryotic gene expression. Nat. Rev. Mol. Cell Biol 23, 93-106 (2022).
34. A. E. A. Hassan, J. Sheng, W. Zhang, Z. Huang, High Fidelity of Base Pairing by 2-Selenothymidine in DNA. J. Am. Chem. Soc. 132, 2120-2121 (2010).
35. J. Y. Wang, J. A. Doudna, CRISPR technology: A decade of genome editing is only the beginning. Science 379, eadd8643 (2023).
36. B. Zetsche et al., Cpf1 Is a Single RNA-Guided Endonuclease of a Class 2 CRISPR-Cas System. Cell 163, 759-771 (2015).
37. T. Yamano et al., Crystal Structure of Cpf1 in Complex with Guide RNA and Target DNA. Cell 165, 949-962 (2016).
38. X. Li et al., Base editing with a Cpf1-cytidine deaminase fusion. Nat. Biotechnol. 36, 324-327 (2018).
39. M. A. McMahon, T. P. Prakash, D. W. Cleveland, C. F. Bennett, M. Rahdar, Chemically Modified Cpf1-CRISPR RNAs Mediate Efficient Genome Editing in Mammalian Cells. Mol. Ther. 26, 1228-1240 (2018).
40. D. Dong et al., The crystal structure of Cpf1 in complex with CRISPR RNA. Nature 532, 522-526 (2016).
41. D. Singh et al., Real-time observation of DNA target interrogation and product release by the RNA-guided endonuclease CRISPR Cpf1 (Cas12a). Proc. Natl. Acad. Sci. U.S.A. 115, 5444-5449 (2018).
42. D. Singh et al., Mechanisms of improved specificity of engineered Cas9s revealed by single-molecule FRET analysis. Nat. Struct. Mol. Biol. 25, 347-354 (2018).
43. C. R. Cromwell et al., Incorporation of bridged nucleic acids into CRISPR RNAs improves Cas9 endonuclease specificity. Nat. Commun. 9, 1448 (2018).
44. D. O'Reilly et al., Extensive CRISPR RNA modification reveals chemical compatibility and structure-activity relationships for Cas9 biochemical activity. Nucleic Acids Res. 47, 546-558 (2019).
45. S. Karmakar, D. Behera, M. J. Baig, K. A. Molla, “In Vitro Cas9 Cleavage Assay to Check Guide RNA Efficiency” in CRISPR-Cas Methods: Volume 2, M. T. Islam, K. A. Molla, Eds. (Springer US, New York, NY, 2021), 10.1007/978-1-0716-1657-4_3, pp. 23-39.
46. E. M. Porto, A. C. Komor, I. M. Slaymaker, G. W. Yeo, Base editing: advances and therapeutic opportunities. Nat. Rev. Drug Discov. 19, 839-859 (2020).
47. M. Jinek et al., A Programmable Dual-RNA& #x2013;Guided DNA Endonuclease in Adaptive Bacterial Immunity. Science 337, 816-821 (2012).
48. H. Nishimasu et al., Crystal Structure of Cas9 in Complex with Guide RNA and Target DNA. Cell 156, 935-949 (2014).
49. M. F. Richter et al., Phage-assisted evolution of an adenine base editor with improved Cas domain compatibility and activity. Nat. Biotechnol. 38, 883-891 (2020).
50. M. Arbab et al., Base editing rescue of spinal muscular atrophy in cells and in mice. Science 380, eadg6518 (2023).
51. J. Grunewald et al., A dual-deaminase CRISPR base editor enables concurrent adenine and cytosine editing. Nat. Biotechnol. 38, 861-864 (2020).
52. E.-A. Raiber, R. Hardisty, P. van Delft, S. Balasubramanian, Mapping and elucidating the function of modified bases in DNA. Nat. Rev. Chem. 1, 0069 (2017).
53. M. K. Bilyard, S. Becker, S. Balasubramanian, Natural, modified DNA bases. Curr. Opin. Chem. Biol. 57, 1-7 (2020).
54. G. Hutinet, Y.-J. Lee, V. de Crécy-Lagard, R. Weigele Peter, D. Hinton, Hypermodified DNA in Viruses of E. coli and Salmonella. EcoSal Plus 9, eESP-0028-2019 (2021).
55. D. A. Malyshev et al., A semi-synthetic organism with an expanded genetic alphabet. Nature 509, 385-388 (2014).
56. Y. Zhang et al., A semi-synthetic organism that stores and retrieves increased genetic information. Nature 551, 644-647 (2017).
57. S. Hoshika et al., Hachimoji DNA and RNA: A genetic system with eight building blocks. Science 363, 884-887 (2019).
58. M. K. Franco, K. S. Koutmou, Chemical modifications to mRNA nucleobases impact translation elongation and termination. Biophys. Chem. https://doi.org/10.1016/j.bpc.2022.106780, 106780 (2022).
59. Y. Hua, T. A. Vickers, B. F. Baker, C. F. Bennett, A. R. Krainer, Enhancement of SMN2 Exon 7 Inclusion by Antisense Oligonucleotides Targeting the Exon. PLOS Biol. 5, e73 (2007).
60. K. M. Brown et al., Expanding RNAi therapeutics to extrahepatic tissues with lipophilic conjugates. Nat. Biotechnol. 40, 1500-1508 (2022).
61. C. Zhu et al., An intranasal ASO therapeutic targeting SARS-CoV-2. Nat. Commun. 13, 4503 (2022).
62. M. J. Mulligan et al., Phase I/II study of COVID-19 RNA vaccine BNT162b1 in adults. Nature 586, 589-593 (2020).
63. L. A. Jackson et al., An mRNA Vaccine against SARS-CoV-2—Preliminary Report. N. Engl. J. Med. 383, 1920-1931 (2020).
64. K. A. Christie et al., Precise DNA cleavage using CRISPR-SpRYgests. Nat. Biotechnol. 41, 409-416 (2023).
65. J. Wang et al., Engineering a PAM-flexible SpdCas9 variant as a universal gene repressor. Nat. Commun. 12, 6916 (2021).
66. S. W. Awwad, A. Serrano-Benitez, J. C. Thomas, V. Gupta, S. P. Jackson, Revolutionizing DNA repair research and cancer therapy with CRISPR-Cas screens. Nat. Rev. Mol. Cell Biol 24, 477-494 (2023).
67. P. D. Donohoue et al., Conformational control of Cas9 by CRISPR hybrid RNA-DNA guides mitigates off-target activity in T cells. Mol. Cell 81, 3637-3649.e3635 (2021).
68. D. E. Ryan et al., Improving CRISPR-Cas specificity with chemical modifications in single-guide RNAs. Nucleic Acids Res. 46, 792-803 (2018).
69. Y. Fu, J. D. Sander, D. Reyon, V. M. Cascio, J. K. Joung, Improving CRISPR-Cas nuclease specificity using truncated guide RNAs. Nat. Biotechnol. 32, 279-284 (2014).
70. D. D. Kocak et al., Increasing the specificity of CRISPR systems with engineered RNA secondary structures. Nat. Biotechnol. 37, 657-666 (2019).
71. I. C. Okafor et al., Single molecule analysis of effects of non-canonical guide RNAs and specificity-enhancing mutations on Cas9-induced DNA unwinding. Nucleic Acids Res. 47, 11880-11888 (2019).
72. H. Park et al., Extension of the crRNA enhances Cpf1 gene editing in vitro and in vivo. Nat. Commun. 9 (2018).
73. D. Mouchiroud et al., The distribution of genes in the human genome. Gene 100, 181-187 (1991).
74. S. Saccone, C. Federico, G. Bernardi, Localization of the gene-richest and the gene-poorest isochores in the interphase nuclei of mammals and birds. Gene 300, 169-178 (2002).
75. D. Kim et al., Genome-wide analysis reveals specificities of Cpf1 endonucleases in human cells. Nat. Biotechnol. 34, 863-868 (2016).
76. B. P. Kleinstiver et al., Genome-wide specificities of CRISPR-Cas Cpf1 nucleases in human cells. Nat. Biotechnol. 34, 869-874 (2016).
77. T. Wang, J. J. Wei, D. M. Sabatini, E. S. Lander, Genetic Screens in Human Cells Using the CRISPR-Cas9 System. Science 343, 80-84 (2014).
78. S. Q. Tsai et al., GUIDE-seq enables genome-wide profiling of off-target cleavage by CRISPR-Cas nucleases. Nat. Biotechnol. 33, 187-197 (2015).
79. Z. Cui et al., Cas13d knockdown of lung protease Ctsl prevents and treats SARS-CoV-2 infection. Nat. Chem. Biol. 18, 1056-1064 (2022).
80. O. S. Akbari et al., Safeguarding gene drive experiments in the laboratory. Science 349, 927-929 (2015).
81. Z. Ye et al., In Vitro Engineering Chimeric Antigen Receptor Macrophages and T Cells by Lipid Nanoparticle-Mediated mRNA Delivery. ACS Biomater. Sci. Eng. 8, 722-733 (2022).
82. T. Matsuda, C. L. Cepko, Electroporation and RNA interference in the rodent retina in vivo and in vitro. Proc. Natl. Acad. Sci. U.S.A. 101, 16-22 (2004).
83. S. Gao et al., Iterative integration of multiple-copy pathway genes in Yarrowia lipolytica for heterologous β-carotene production. Metab. Eng. 41, 192-201 (2017).
84. S. Hirano et al., Structural basis for the promiscuous PAM recognition by Corynebacterium diphtheriae Cas9. Nat. Commun. 10, 1968 (2019).
85. H. M. Park et al., Extension of the crRNA enhances Cpf1 gene editing in vitro and in vivo. Nat. Commun. 9, 3313 (2018).
86. K. Clement et al., CRISPResso2 provides accurate and rapid genome editing sequence analysis. Nat. Biotechnol. 37, 224-226 (2019).
87. G.-H. Hwang et al., Web-based design and analysis tools for CRISPR base editing. BMC Bioinform. 19, 542 (2018).
88. Scholz, I., S. J. Lange, S. Hein, W. R. Hess, and R. Backofen. 2013. ‘CRISPR-Cas systems in the cyanobacterium Synechocystis sp. PCC6803 exhibit distinct processing pathways involving at least two Cas6 and a Cmr2 protein’, PLOS One, 8: e56470.
89. Pattharaprachayakul, Napisa, Mieun Lee, Aran Incharoensakdi, and Han Min Woo. 2020. ‘Current understanding of the cyanobacterial CRISPR-Cas systems and development of the synthetic CRISPR-Cas systems for cyanobacteria’, Enzyme and Microbial Technology, 140:109619.

### INCORPORATION BY REFERENCE

All U.S. patents and U.S. and PCT published patent applications mentioned herein are hereby incorporated by reference in their entirety as if each individual patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A nucleic acid, comprising less than or equal to 2,500 nucleotides, wherein at least 15% of said nucleotides comprise a 2-aminoadenine (Z) base.

2. The nucleic acid, comprising less than or equal to 2,000 nucleotides.

3. The nucleic acid of claim 1 or 2, wherein at least 38% or at least 39% of said nucleotides comprise a Z base.

4. The nucleic acid of any one of claims 1-3, wherein the nucleic acid does not comprise an adenine (A) base.

5. The nucleic acid of any one of claims 1-4, wherein the nucleic acid comprises at least 100 or at least 160 nucleotides.

6. The nucleic acid of any one of claims 1-5, wherein the nucleic acid is a DNA comprising at least one intron, a cDNA, or an mRNA.

7. The nucleic acid of any one of claims 1-6, wherein the nucleic acid is an mRNA comprising a poly(Z) tail.

8. The nucleic acid of any one of claims 1-7, further comprising at least one chemical modification.

9. A vector, comprising a nucleic acid of any one of claims 1-8.

10. The vector of claim 9, wherein the vector is an expression vector.

11. A method of expressing or producing a protein, comprising contacting a cell with a nucleic acid of any one of claims 1-8, or with a vector of claim 9 or 10.

12. The method of claim 11, wherein the cell is a prokaryotic cell or a eukaryotic cell.

13. A composition or kit, comprising:
(a) a Cas12a RNA-guided endonuclease or a nucleic acid encoding the Cas12a RNA-guided endonuclease; and
(b) a Cas12a crRNA comprising at least one Z base.

14. The composition or kit of claim 13, wherein the Cas12a crRNA comprises at least 7 Z bases.

15. The composition or kit of claim 13 or 14, wherein the Cas12a crRNA does not comprise an A base.

16. The composition or kit of any one of claims 13-15, wherein the Cas12a RNA-guided endonuclease is LbCas12a (LbCpf1).

17. A method of cleaving or modifying a target DNA, comprising:
contacting the target DNA or a cell comprising the target DNA with:
(a) a Cas12a RNA-guided endonuclease, or a nucleic acid encoding the Cas12a RNA-guided endonuclease; and
(b) a Cas12a crRNA comprising at least one Z base,
wherein the Cas12a RNA-guided endonuclease and the Cas12a crRNA form a complex that cleaves or modifies the target DNA.

18. The method of claim 17, wherein the Cas12a crRNA comprises at least 7 Z bases.

19. The method of claim 17 or 18, wherein the Cas12a crRNA does not comprise an A base.

20. The method of any one of claims 17-19, wherein the Cas12a RNA-guided endonuclease is LbCas12a.

21. The method of any one of claims 17-20, wherein the target DNA is a plasmid DNA.

22. The method of any one of claims 17-21, wherein the Cas12a crRNA comprising at least one Z base induces higher cleavage of the target DNA compared to the corresponding Cas12a crRNA wherein each of the at least one Z base is substituted with an A base.

23. The method of claim 22, wherein the Cas12a crRNA comprising at least one Z base induces at least 1.1-fold cleavage of the target DNA compared to the corresponding Cas12a crRNA wherein each of the at least one Z base is substituted with an A base.

24. The method of claim 23, wherein the Cas12a crRNA comprising at least one Z base induces 1.2-fold, 1.4-fold, 1.8-fold, 3.3-fold, or 6-fold cleavage of the target DNA compared to the corresponding Cas12a crRNA wherein each of the at least one Z base is substituted with an A base.

25. The method of any one of claims 17-24, wherein the Cas12a crRNA comprises at least one Z base in the seed region.

26. The method of any one of claims 17-25, wherein the Cas12a crRNA comprises a 20-nt guide sequence complimentary to a target DNA site with an A or Z content from 15% to 35%.

27. The method of any one of claims 17-26, wherein the Cas12a crRNA comprises a 20-nt guide sequence complimentary to a target DNA site with an AT or ZT content from 45% to 75%, or from 45% to 85%.

28. The method of any one of claims 17-27, wherein the target DNA comprises a 5′-TTTA, 5′-TTTC, or 5′-TTTG PAM motif.

29. The method of any one of claims 17-28, wherein the target DNA comprises a T-base at position 5′-PAM-6, 8, 10, 18, 19-3′.

30. The method of any one of claims 17-29, wherein the target DNA comprises at least one Z base.

31. A method of improving cleavage activity or editing efficiency of a complex comprising a Cas12a RNA-guided endonuclease and a Cas12a crRNA, comprising substituting at least one A base of the Cas12a crRNA with a Z base.

32. The method of claim 31, comprising substituting all A bases of the Cas12a crRNA with Z bases.

33. The method of claim 31 or 32, comprising substituting at least one A base of the DNA substrate with a Z base.

34. The method of any one of claims 31-33, comprising substituting all A bases of the DNA substrate with Z bases.

35. A method of cleaving or modifying a target DNA comprising at least one Z base, comprising:
contacting the target DNA or a cell comprising the target DNA with:
(a) a Cas12a RNA-guided endonuclease or a nucleic acid encoding the Cas12a RNA-guided endonuclease; and
(b) a Cas12a crRNA or a nucleic acid encoding a Cas12a crRNA,
wherein the Cas12a RNA-guided endonuclease and the Cas12a crRNA form a complex that cleaves or modifies the target DNA comprising at least one Z base.

36. A composition or kit, comprising:
(a) a Cas9 protein or a nucleic acid encoding the Cas9 protein;
(b) a Cas9 crRNA comprising at least one Z base; and
(c) a Cas9 tracrRNA.

37. The composition or kit of claim 36, wherein the Cas9 crRNA comprises at least 12 Z bases.

38. The composition or kit of claim 36 or 37, wherein the Cas9 crRNA does not comprise an A base.

39. The composition or kit of any one of claims 36-38, wherein the Cas9 crRNA comprises a 20-nt guide sequence complimentary to a target DNA site with an A or Z content from 35% to 60%.

40. The composition or kit of any one of claims 36-39, wherein the Cas9 crRNA comprises a 20-nt guide sequence complimentary to a target DNA site with an AT or ZT content from 45% to 90%.

41. The composition or kit of any one of claims 36-49, wherein the Cas9 tracrRNA comprises at least one Z base.

42. The composition or kit of any one of claims 36-41, wherein the Cas9 tracrRNA does not comprise an A base.

43. A method of cleaving or modifying a target DNA, comprising:
contacting the target DNA or a cell comprising the target DNA with:
(a) a Cas9 protein, or a nucleic acid encoding the Cas9 protein;
(b) a Cas9 crRNA comprising at least one Z base, and
(c) a Cas9 tracrRNA;
wherein the Cas9 protein, the Cas9 crRNA, and the Cas9 tracrRNA form a complex that cleaves or modifies the target DNA.

44. The method of claim 43, wherein the Cas9 crRNA comprises at least 12 Z bases.

45. The method of claim 43 or 44, wherein the Cas9 crRNA does not comprise an A base.

46. The method of any one of claims 43-45, wherein the Cas9 crRNA comprises a 20-nt guide sequence complimentary to a target DNA site with an A or Z content from 35% to 60%.

47. The method of any one of claims 43-46, wherein the Cas9 crRNA comprises a 20-nt guide sequence complimentary to a target DNA site with an AT or ZT content from 45% to 90%.

48. The method of any one of claims 43-47, wherein the Cas9 tracrRNA comprises at least one Z base.

49. The method of any one of claims 43-48, wherein the Cas9 tracrRNA does not comprise an A base.

50. The method of any one of claims 43-49, wherein the target DNA is a plasmid DNA.

51. The method of any one of claims 43-50, wherein the target DNA comprises at least one Z base.

52. A method of cleaving or modifying a target DNA comprising at least one Z base, comprising:
contacting the target DNA with:
(a) a Cas9 protein or a nucleic acid encoding the Cas9 protein; and
(b) a Cas9 guide RNA (gRNA) or a nucleic acid encoding a Cas9 gRNA,
wherein the Cas9 protein and the Cas9 gRNA form a complex that cleaves or modifies the target DNA comprising at least one Z base.

53. The method of claim 52, wherein the target DNA comprises a Z base in a spacer region or a PAM region or both.

54. The method of claim 52 or 53, wherein the target DNA is a plasmid DNA.

55. A method of modifying a target DNA comprising:
contacting the target DNA or a cell comprising the target DNA with:
(a) a Cas9-guided base editor or a nucleic acid encoding the Cas9-guided base editor; and
(b) a Cas9 crRNA comprising at least one Z base; and
(c) a Cas9 tracrRNA,
wherein the Cas9-guided base editor, the Cas9 crRNA, and the Cas9 tracrRNA form a complex that induces a base change of the target DNA.

56. The method of claim 55, wherein the Cas9-guided base editor is an adenine base editor (ABE).

57. The method of claim 55 or 56, wherein the Cas9-guided base editor is ABE8e.

58. The method of any one of claims 55-57, wherein the Cas9-guided base editor, the Cas9 crRNA, and the Cas9 tracrRNA form a complex that induces a A-to-G change of the target DNA.

59. The method of claim 58, wherein the Cas9-guided base editor, the Cas9 crRNA, and the Cas9 tracrRNA form a complex that induces A-to-G changes with a frequency of at least 5%.

60. The method of any one of claims 55-59, wherein the Cas9 crRNA comprises at least 8 Z bases.

61. The method of any one of claims 55-60, wherein the Cas9 crRNA does not comprise an A base.

62. The method of claim 61, wherein the cell is a mammalian cell.
